(19) United States
(12) Patent Application Publication
(10) Pub. No.: US 2017/0314056 A1
(43) Pub. Date: Nov. 2, 2017

(54) METHOD OF USING NEAR INFRARED FLUORESCENT DYES FOR IMAGING AND TARGETING CANCERS

(71) Applicants: Xiaojian Yang, Xi'an (CN); Haiyen E. Zhau, Beverly Hills, CA (US); Ruoxiang Wang, Los Angeles, CA (US); Leland W.K. Chung, Beverly Hills, CA (US)

(72) Inventors: Xiaojian Yang, Xi'an (CN); Haiyen E. Zhau, Beverly Hills, CA (US); Ruoxiang Wang, Los Angeles, CA (US); Leland W.K. Chung, Beverly Hills, CA (US)

(21) Appl. No.: 15/487,320

(22) Filed: Apr. 13, 2017

Related U.S. Application Data
(63) Continuation of application No. 13/634,677, filed on Nov. 21, 2012, now abandoned.
(60) Provisional application No. 61/370,399, filed on Aug. 3, 2010.
(60) Provisional application No. 61/316,771, filed on Mar. 23, 2010.
(60) Provisional application No. 61/314,534, filed on Mar. 16, 2010.

(51) Int. Cl. C12Q 1/04; G01N 33/50; G01N 21/64; C09B 23/08; A61B 5/00; C09B 23/01; A61K 49/00; G01N 33/574

(52) U.S. Cl. CPC C12Q 1/04; G01N 33/574; G01N 33/5091; G01N 21/6428; C09B 23/086; C09B 23/0066; A61B 5/0059; C09B 23/0016; A61K 49/006; C09B 23/0033; A61B 5/417; A61B 2503/40

(57) ABSTRACT

The present invention describes methods of identifying, detecting, imaging, isolating and locating cancer cells in a subject. The method involves the use of near-infrared (NIR) organic carbocyanine dyes, particularly, near infrared heptamethine cyanine dyes and the detection of the fluorescence of these NIR dyes. The uptake of these dyes by cancer cells and not by normal cells, as well as their high intensity, among other things, allow for the detection of cancerous cells in a subject and facilitate their subsequent isolation. Further, detection of many tumor types and tumor cell populations under cell culture and in vivo conditions are described.

### CROSS-REFERENCE TO RELATED APPLICATIONS

This application is a Continuation to U.S. application Ser. No. 13/634,677, filed Nov. 21, 2012, which is a National Phase of International Application No. PCT/US2011/028738, filed Mar. 16, 2011, which designated the U.S. and that International Application was published under PCT Article 21 (2) in English. This application also includes a claim of priority under 35 U.S.C. §119(e) to U.S. provisional patent application No. 61/314,534 filed Mar. 16, 2010, U.S. provisional patent application No. 61/316,771 filed Mar. 23, 2010 and U.S. provisional patent application No. 61/370,399 filed Aug. 3, 2010.

### STATEMENT REGARDING FEDERALLY-SPONSORED RESEARCH

The U.S. Government has a paid-up license in this invention and the right in limited circumstances to require the patent owner to license others on reasonable terms as provided for by the terms of Grant Nos. 1P50 CA128301 and 1 U54 CA119338 awarded by the National Institutes of Health.

### FIELD OF INVENTION

This invention relates to imaging methods for detecting tumor cells and imaging cells of interest.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

The future of personalized oncology and medicine relies on the improvement of imaging methods for detecting tumor cells and metastatic deposits earlier in patients, by providing a sensitive imaging probe for real-time support for surgeons, oncologists, pathologists and laboratory medicine personnel. They also require the development of novel methods that can quantify drugs directly for primary and metastatic tumor sites and guide designated drugs, radionuclides, substrates, metabolites, genes, gene transcripts, gene modifiers, or gene products via chemical conjugation or complex formation of the said molecules with organic carbocyanine dyes to interfere with the behaviors of the tumor cells.

Cancer mortality can be reduced by the development of non-invasive and effective imaging technologies that can detect tumors at metastatic sites and cancer cells in biologic fluids. Near-infrared (NIR) excitable fluorescent contrast agents offer unique possibilities for in vivo cancer imaging. These agents show little autofluorescence in aqueous solution, and upon binding to macromolecules in cells, NIR carbocyanine dyes display drastically increased fluorescence due to rigidization of the fluorophores (1). The most common NIR fluorophores are polymethine cyanine dyes. In clinical practice, pentamethine and heptamethine cyanines comprised of benzoxazole, indole, and quinoline are of great value and interest (2, 3). These organic dyes are characterized by high extinction coefficients and relatively large Stokes' shifts. With emission profiles at 700-1000 nm, their fluorescence can be readily detected from deep tissues by commercially available imaging modalities (4-6).

Application of organic dyes in cancer detection and diagnosis has yet to be fully explored (5). The conventional approach to tumor imaging is through designed delivery of NIR fluorophores, mostly by chemical conjugation to tumor-specific ligands including metabolic substrates, aptamers, growth factors, and antibodies (7-10). A number of surface molecules have been tested as targets, including membrane receptors, extracellular matrices, cancer cell-specific markers and neovascular endothelial cell-specific markers (11-13). One limitation of these approaches is that the previous NIR moieties only detect specific cancer cell types with well-characterized surface properties, whereas tumors are notorious for their heterogeneity (14, 15). In addition, chemical conjugation may alter the specificity and affinity of the targeting ligands (3). A simpler and more straightforward strategy is needed to broaden the use of NIR dyes for non-invasive tumor imaging.

National Cancer Institute predicts a person at age zero has a 40.77% lifetime risk of being diagnosed with cancer and a 21.15% lifetime risk of dying from cancer in the United States. With respect to prostate cancer, the lifetime risk of being diagnosed with and dying from cancer are 16.22% and 2.79%, respectively. With respect to renal cancer and cancer of the renal pelvis, the risks are 1.49% and 0.47%, respectively.

Patients with metastatic disease still have an extremely short life expectancy.⁵² Thus, diagnosis of small premalignant lesions and early-stage primary tumors is crucial for the success of renal cancer therapy and increases survival rates. Interest has increased within the past decade in fluorescence-based and other optical imaging techniques for clinical diagnosis.

Accordingly, there exists a need in the art for methods that will to allow for improved detection of cancer cells, permit personalized oncology and as well as improvements for medical treatments.

### SUMMARY OF THE INVENTION

The following embodiments and aspects thereof are described and illustrated in conjunction with compositions and methods which are meant to be exemplary and illustrative, not limiting in scope.

Various embodiments of the present invention provide for a method, comprising: providing a biological sample from a subject; contacting the biological sample with a composition comprising a near-infrared (NIR) organic carbocyanine dye to form a mixture; and analyzing the mixture to identify, detect, locate, isolate and/or characterize a possible cancer cell or tumor in the biological sample.

In various embodiments, the biological sample can be selected from the group consisting of tissue, tumor tissue, cancer tissue, cell, tumor cell, cancer cell, body fluid, whole blood, plasma, stool, intestinal fluid or aspirate, stomach fluid or aspirate, serum, cerebral spinal fluid (CSF), urine, sweat, saliva, tears, pulmonary secretion, breast aspirate, breast milk, prostate fluid, seminal fluid, cervical scraping, bone marrow aspirate, amniotic fluid, intraocular fluid, mucous, moisture in breath.

In various embodiments, the method can identify or detect the presence of a cancer cell or a tumor in the biological sample when a presence of an increased NIR fluorescent signal, relative to a background staining intensity, is detected from the cell or tumor in the biological sample; and can identify or detect the absence of a cancer cell or a tumor in the biological sample when there is an absence of increased NIR fluorescent signal, relative to a background staining intensity, from the cell or tumor in the biological sample. In various embodiments, method can locate the a cancer cell or a tumor in the biological when a presence of an increased NIR fluorescent signal, relative to a background staining intensity, is detected from the cell or tumor in the biological sample. In various embodiments, the method can isolate a cancer cell or a tumor from the biological by: detecting a presence of an increased NIR fluorescent signal, relative to a background staining intensity, from the cell or tumor in the biological sample; and separating the cell or tumor from the biological sample based on the increased NIR fluorescent signal. In various embodiments, the method can characterize a cancer cell or tumor in the biological sample by: determining the concentration of the NIR fluorescent dye in the cancer cell or tumor.

In various embodiments, analyzing the mixture can be performed by using a flow cytometer, by using fluorescent microscopy, or by using fluorescence activated cell sorting (FACS).

In various embodiments, the cancer cell or tumor can be a type selected from the group consisting of local and disseminated prostate, breast, lung, cervical, skin, renal, leukemia, bladder, osteosarcoma and combinations thereof. In various particular embodiments, the cancer can be prostate cancer. In various particular embodiments, the cancer can be renal cancer. In various embodiments, the cancer cell or tumor can be a metastasized cancer cell or tumor.

In various embodiments, the NIR organic carbocyanine dye can be an NIR heptamethine cyanine dye. In various embodiments, the NIR organic carbocyanine dye can be IR-780, IR-783, or MHI-148.

In various embodiments, analyzing the biological sample can comprise imaging the mixture.

In various embodiments, imaging can be performed about 24 to 48 hours after contacting the NIR organic carbocyanine dye to the sample, or can be performed about 48 to 96 hours after contacting the NIR organic carbocyanine dye to the sample.

In various embodiments the tumor identified, detected, located, isolated, and/or characterized can be less than 1 mm³. In various embodiments, at least 1 cancer cell is identified, detected, located, isolated and/or characterized from a sample comprising 10 cancer cells/ml.

In various embodiments, the biological sample can be a formalin or water soluble chemically fixed tissue sample, or a frozen section of tissue specimen, and the method can detects the presence of trace amounts of cancer or tumor cells.

Various embodiments of the present invention provide a method, comprising: providing a composition comprising a near-infrared (NIR) organic carbocyanine dye; administering composition comprising the NIR organic carbocyanine dye to a subject in need thereof; and imaging the subject to identify, detect, image, locate, and/or characterize a cancer cell or tumor in the subject.

In various embodiments, the cancer cell or tumor can be a type selected from the group consisting of local and disseminated prostate, breast, lung, cervical, skin, renal, leukemia, bladder, osteosarcoma and combinations thereof. In various particular embodiments, the cancer can be prostate cancer or renal cancer. In various embodiments, the cancer cell or tumor can be a metastasized cancer cell or tumor.

In various embodiments, the NIR organic carbocyanine dye can be an NIR heptamethine cyanine dye. In various embodiments, the NIR organic carbocyanine dye can be IR-780, IR-783, or MHI-148.

In various embodiments, the cancer cell or tumor is identified or detected in the subject, the cancer cell or tumor is located in the subject, or the cancer cell or tumor is characterized in the subject.

In various embodiments, the presence of an increased NIR fluorescent signal, relative to the background staining intensity, can indicate the cell is a cancer or tumor cell, and the lack of an increased NIR fluorescent signal, relative to the background staining intensity can indicate that the cell is not a cancer or tumor cell.

In various embodiments, imaging the subject can be performed about 24 to 48 hours after administering the NIR organic carbocyanine dye or can be performed about 48 to 96 hours after administering the NIR organic carbocyanine dye.

In various embodiments, the tumor identified, detected, imaged, located, and/or characterized can be less than 1 mm³. In various embodiments, at least 1 cancer cell can be identified, detected, located, isolated and/or characterized in a subject who has 10 circulating cancer cells per ml of blood.

In various embodiments, the method can further comprise merging a fluorescence image obtained from imaging the subject with an x-ray image of the subject.

Various embodiments of the present invention provide a method of isolating a cancer cell in a subject in need thereof, comprising: providing a biological sample from the subject; contacting the biological sample with a composition comprising a near-infrared (NIR) organic carbocyanine dye; detecting a NIR fluorescent signal in cell in the biological sample, wherein the presence of an increased NIR fluorescent signal, relative to the background staining intensity, indicates the cell is a cancer or tumor cell, and the lack of an increased NIR fluorescent signal, relative to the background staining intensity indicates that the cell is not a cancer or tumor cell; and separating a cell possessing the NIR fluorescent signal from the biological sample.

In various embodiments, a microfluidic apparatus or a flow cytometer can be used to detect the fluorescence. In various embodiments, a fluorescence activated cell sorting (FACS) system can be used to detect the fluorescence in a cell and to separate a cancer or tumor cell from the biological sample.

Various embodiments of the present invention provide a method, comprising: providing a composition comprising a near-infrared (NIR) organic carbocyanine dye conjugated or complexed to a molecule; administering the composition comprising the NIR organic carbocyanine dye-molecule conjugate or complex to a subject; and (i) determining the pharmacokinetics and/or pharmacodynamics of the molecule in a cancer cell or tumor cell in the subject, (ii) imaging the subject to follow the movement of the molecule in the subject, or (iii) increasing the delivery of the molecule to a cancer cell or tumor cell in the subject.

Various embodiments of the present invention provide for a method, comprising: providing a composition comprising a near-infrared (NIR) organic carbocyanine dye; contacting the composition comprising the NIR organic carbocyanine dye to a cancer cell or a tumor cell to allow uptake of the NIR organic carbocyanine dye; administering the cancer cell or the tumor cell containing the NIR organic carbocyanine dye to a subject; and imaging the subject to follow the movement of the cell in the subject.

Various embodiments of the present invention provide for a method, comprising: providing composition comprising a near-infrared (NIR) organic carbocyanine dye; administering the composition comprising the NIR organic carbocyanine dye a subject; and (i) imaging the subject to follow and/or study the metastasis of a cancer cell or tumor cell, or (ii) imaging the subject to detect vascularization changes in a tumor.

Various embodiments of the present invention provide for a method, comprising: providing a composition comprising a near-infrared (NIR) organic carbocyanine dye; contacting the composition comprising the NIR organic carbocyanine dye to a biological sample comprising cancer or tumor cells; and imaging the biological sample to differentiate live cells versus dead cells.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts the chemical structure of IR-783 in accordance with various embodiments of the present invention.

FIG. 2 depicts the absorption and emission spectra of IR-783 in accordance with various embodiments of the present invention.

FIG. 3 depicts the chemical structure of IR-780 in accordance with various embodiments of the present invention.

FIG. 4 depicts active uptake of IR-780 by human cancer cells in culture in accordance with various embodiments of the present invention. Human cancer cells including prostate (ARCaP_{M}, C-2, PC-3), liver (HepG2), breast (MCF-7), kidney (RCC), bladder (T-24), cervical (HeLa), leukemia (K562), and lung (H358) cancer cells showed a significant uptake of IR-780, while normal prostate epithelial cells (NPE), marrow stromal cells (BMC) showed a very low uptake of this organic carbocyanine dye in culture. All the cells were cultured with 20 μM IR-780 in basal media (T-medium with 5% fetal bovine serum and 1% antibiotics) for 30 minutes and were imaged under confocal microscope. Similar results were obtained when IR-783 organic dye was used (data not shown).

FIG. 5 depicts human renal cancer cells (SN12C) actively taking up IR-783 in accordance with various embodiments of the present invention. Control gave background activity.

FIG. 6 depicts human renal cancer cells (ACHN) taking up the dye in accordance with various embodiments of the present invention.

FIG. 7 depicts human bladder cancer cell lines showing strong uptake of IR-783 in accordance with various embodiments of the present invention.

FIG. 8 depicts human pancreatic cancer cell lines showing strong uptake of IR-783 in accordance with various embodiments of the present invention.

FIG. 9 depicts human embryonic kidney epithelial normal cells yielding low uptake of IR-783 in accordance with various embodiments of the present invention.

FIG. 10 depicts time-dependent uptake of NIR dye in prostate cancer but not normal prostate epithelial cells in accordance with various embodiments of the present invention. ARCaP_{M} cancer cells and normal prostate epithelial cells (P69) were plated on live-cell imaging chambers from World Precision Instrument (Sarasota, Fla.) overnight. Cells were treated with 20 μM IR-780 and cells were imaged using a Perkin-Elmer Ultraview ERS spinning disc confocal microscope. As shown, there is a big difference in IR-780 uptake by cancer cells and normal cells.

FIG. 11 depicts time- and concentration-dependent uptake of NIR dye in accordance with various embodiments of the present invention. ARCaP_{M} Cells were plated on live-cell imaging chambers from World Precision Instrument (Sarasota, Fla.) overnight. Cells were treated with IR-780 with different concentrations and were imaged using a Perkin-Elmer Ultraview ERS spinning discconfocal microscope. This system was mounted on a Zeiss Axiovert 200m inverted microscope equipped with a 37° C. stage warmer, incubator, and CO₂ perfusion. A x63 or x100 Zeiss oil objective (numerical aperture, 1.4) was used for all images and a Z-stack was created using the attached piezo electric z-stepper motor. The 633 nm laser line of an argon ion laser (set at 60% power) was used to excite the IR-780 organic dye. For each comparison, the exposure time and laser intensity was kept identical for accurate intensity measurement. Pixel intensity was quantitated using Metamorph 6.1 (Universal Imaging, Downingtown, Pa.) and the mean pixel intensity was generated as grey level using the Region Statistics feature on the software.

FIG. 12 shows that the uptake of NIR dye by ARCaP_{M} cells is blocked by BSP, an organic anion transporter inhibitor in accordance with various embodiments of the present invention. ARCaP_{M} Cells were plated on live-cell imaging chambers from World Precision Instrument (Sarasota, Fla.) overnight. Cells were treated with 20 μM IR-780 and 250 μM BSP at the same time and cells were imaged using a Perkin-Elmer Ultraview ERS spinning disc confocal microscope. As shown, BSP significantly inhibited the uptake of IR-780 by cancer cells.

FIG. 13 depicts NIR dye co-localizing with mitochondrial, lysosomal and cytoplasmic compartments in accordance with various embodiments of the present invention. ARCaP_{M} cells were co-stained with IR-780 and mitochondrial tracker or lysosome tracker and were imaged under confocal microscope. Co-localization of IR-780 and mitochondrial and lysosome were shown.

FIG. 14 shows that normal mouse tissue does not uptake the dye in accordance with various embodiments of the present invention. One mouse was sacrificed at 96 hrs after IR-780 injection through tail vein, and isolated tissues and organs excised from mice were cut into frozen slides and were imaged under confocal microscope. Note that all mouse tissues failed to uptake this organic dye. These results are in agreement with the whole animal study where no detectable dye was associated with normal tissues.

FIG. 15 shows uptake of IR-783 in accordance with various embodiments of the present invention. Subcutaneous ARCaP_{M} and PC3 (four tumors with different sizes) tumors were established in live mice prior to the administration of IR-783, 5 nmol of IR-783 were given to mice through tail vein and imaged using Kodak muitimodal-imaging system. As shown, clear tumor images can be detected in both subcutaneous ARCaP_{M} and PC3 tumors following intravenous administration of IR-783.

FIG. 16 depicts strong uptake of IR-783 by orthotopic ARCaP_{M} tumors in accordance with various embodiments of the present invention.

FIG. 17 depicts the uptake of IR-780 in accordance with various embodiments of the present invention. Intratibial ARCaP_{M} tumors in live mice were established prior to the administration of IR-780. 5 nmol of IR-780 were given to mice through tail vein and imaged using Kodak multimodal-imaging system. As shown, clear tumor images can be detected following intravenous administration of IR-780. The distribution of IR-780 in tumor tissues was confirmed by imaging of frozen sections under confocal microscope. The tumor was confirmed by H/E staining.

FIG. 18 depicts IR-780 uptake in accordance with various embodiments of the present invention. Subcutaneous HepG2 (human liver cancer), C4-2 (human prostate androgen-independent and metastatic cancer), H358 (human lung cancer), HeLa (human cervical cancer), MCF-7 (human breast cancer) and ARCaP_{M} (a highly bone and soft tissue metastatic human prostate cancer) cancers were established in live mice prior to the administration of IR-780. The tumor xenografts were measured about 0.5-1.0 cm in diameter at the time of imaging. 5 nmol of IR-780 were given to mice through tail vein and imaged using Kodak multimodal-imaging system. As shown, clear tumor images can be detected in all subcutaneous tumors following intravenous administration of IR-780 with no back ground autofluorescence. The distribution of IR-780 in tumor tissues was confirmed by imaging of frozen sections under confocal microscope. The histopathology of the tumor was confirmed by H/E staining. DAPI stained cell nuclei.

FIG. 19 shows that orthotopic ARCaP and its metastases also show active uptake of IR-783 in accordance with various embodiments of the present invention.

FIG. 20 depicts human bladder carcinoma subcutaneous implants imaging by IR-783 in accordance with various embodiments of the present invention. Results were confirmed by histopathology.

FIG. 21 depicts uptake of the NIR dye by pancreatic cancer cell SQ (PDAC3.3) in accordance with various embodiments of the present invention.

FIG. 22 depicts uptake of the NIR dye by pancreatic cancer cell SQ (PDAC2.3) in accordance with various embodiments of the present invention.

FIG. 23 depicts transgenic mice bearing prostate tumors (TRAMP) also showing active uptake of IR-783 dye in accordance with various embodiments of the present invention. Tumors were present in both primary and metastatic sites.

FIG. 24 depicts a time-course of IR-783 uptake by ARCaP orthotopic tumors in accordance with various embodiments of the present invention. Note 24-48 hours after dye uptake, tumors and metastases can be readily identified.

FIG. 25 depicts ARCaP orthotopic tumors uptake IR-783 dye in a time-dependent manner with tumor and metastases identified in accordance with various embodiments of the present invention.

FIG. 26 depicts IR-783 dye uptake by orthotopic ARCaP tumor and its distant metastases to soft tissues in accordance with various embodiments of the present invention. The metastases were confirmed by H/E histopathologic section.

FIG. 27 depicts IR-783 dye uptake by orthotopic ARCaP tumor and its distant metastases to soft tissues in another mouse in accordance with various embodiments of the present invention. The metastases were confirmed by H/E histopathologic section.

FIG. 28 depicts ARCaP bone metastases imaging by IR-783 in accordance with various embodiments of the present invention. Results were confirmed by histopathology.

FIG. 29 depicts the results compared for a 28-day period of mice injected by 100× of the imaging dose of IR-783 and IR-780 in accordance with various embodiments of the present invention. Note IR-783 was not toxic with mice gaining weight like the controls (PBS injected) during this observation period. IR-780 was toxic, killing all mice 2 days after the dye injection.

FIG. 30 depicts uptake of IR-783 by five freshly isolated human renal tumor specimens in accordance with various embodiments of the present invention. Note strong IR-783 uptake in tumor but not in normal kidney tissues. The dye uptake was confirmed by confocal microscopic evaluation of frozen tissue specimens.

FIG. 31 depicts uptake of NIR dye in human renal tumors in accordance with various embodiments of the present invention.

FIG. 32 depicts uptake of NIR dye in additional human renal tumors in accordance with various embodiments of the present invention.

FIG. 33 depicts uptake of NIR dye also in additional human renal tumors in accordance with various embodiments of the present invention.

FIG. 34 depicts uptake of NIR dye in human renal tumors in accordance with various embodiments of the present invention.

FIG. 35 depicts uptake of the IR-783 dye in renal cancer tissue implants in mice in accordance with various embodiments of the present invention.

FIG. 36 depicts uptake of IR-783 dye in human bladder tumors in accordance with various embodiments of the present invention. IR-783 was taken up by tumor tissues and to a much lesser extent by normal tissue. IR-783 was also taken up by fat cells and tissues.

FIG. 37 depicts the comparison of luminescent imaging and IR-783 imaging in accordance with various embodiments of the present invention. Results show that IR-783 has the advantage over that of the luminescent imaging by providing anatomical information when used in combination with x-ray in Kodak imaging system.

FIG. 38 depicts active uptake of heptamethine cyanine dyes by human cancer cells but not normal cells in culture in accordance with various embodiments of the present invention. Panels A, B, and C are described below. (A) The chemical structures of two heptamethine cyanine dyes, IR-783 and MHI-148. (B) Normal human cells including bone marrow stromal cells (HS-27A), normal prostate epithelial cells (NPE), normal prostate stromal fibroblasts (NPF), vascular endothelial cells (HUVEC-CS) and human embryonic kidney cells (HEK293) showed very low uptake of these dyes in culture. (C) Human cancer cell lines including prostate (C4-2, PC-3, ARCaP_{M}), breast (MCF-7), cervical (HeLa), lung (H358), liver (HepG2), pancreatic (MIA PaCa-2) and renal (SN12C) cancer cells, as well as a human leukemia cell line (K562), showed significant uptake of IR-783 dye under similar staining and imaging conditions. Results are shown with images obtained from cells stained with DAPI of cell nuclei, the heptamethine cyanine IR-783 stain (NIR), and a merger of the two images (Merge). All the images were acquired at 630× magnification.

FIG. 39 depicts kinetics and subcellular localization of the NIR dyes in accordance with various embodiments of the present invention. Panels A, B, C, and D, are described below. (A) Confocal imaging shows significant uptake of IR-783 dye in ARCaP_{M} cells but not in normal human prostate epithelial P69 cells at 630× magnification. (B) Histogram shows differential and time-dependent uptake of IR-783 by human prostate cancer ARCaP_{M} cells and P69 cells. (C) Uptake of the IR-783 dye (20 μM) by ARCaP_{M} cells can be abrogated by 250 μM BSP. (D) Subcellular co-localization of the NIR heptamethine cyanine dyes with lysosomes (Lyso) and mitochondrial (Mito) tracking dyes. ARCaP_{M} cells that were stained with IR-783 were stained with a lysosome-specific dye, Lyso Tracking Green DND-26, and a mitochondria-specific dye, Mito Tracker Orange CMTMROS (630×). Fluorescence imaging indicates that a large portion of the IR-783 was co-localized with these subcellular organelles.

FIG. 40 depicts preferential uptake and retention of the heptamethine cyanine dyes in human tumor xenografts in accordance with various embodiments of the present invention. Mice bearing human prostate (ARCaP_{M}, orthotopic prostate tumor, p.o), bladder (T24, subcutaneous, s.c.), pancreatic (MIA PaCa-2, subcutaneous), and renal (SN12C, intraosseous to tibia, i.o.) tumors were injected i.p. with IR-783 at a dose of 10 nmol/20 g. NIR imaging was performed 24 hrs later. Each mouse was subjected to fluorescence imaging (NIR) and X-ray imaging (X-ray) using the Kodak Imaging Station Imaging System, and the two images were superimposed (Merge) for tumor localization. After imaging, tissues with specific fluorescence signals were dissected, fixed in 10% formaldehyde, and subjected to histopathologic analysis by H/E staining (200×). In mice bearing subcutaneous tumors, both tumors were detected based on fluorescence imaging (see arrows).

FIG. 41 depicts detection of tumor metastasis in mice and spontaneous tumors in transgenic animals in accordance with various embodiments of the present invention. Panels A (with subpanels a, b, c, and d), B, and C are described below. (A) Confirmation of the presence of bone metastatic prostate tumors in mice by NIR imaging after IR-783 i.p injection at a dose of 10 nmol/20 g. (a) The ARCaP_{M} human prostate cancer cell line was stably transfected with AsRed2 RFP. The clone being used in this study exhibited typical ARCaP_{M} cell morphology (bright field, 100×) and could emit intense red fluorescence. (b) Cells from this clone were inoculated orthotopically to athymic mice to produce both localized prostate tumor (thick arrow) and bone metastatic tumor (thin arrow), which were detected by IR-783 fluorescence imaging of the whole animal (left) and of the dissected skeletal bone (right). (c) To confirm the detection of metastasis, marrow cells from the affected tibia/femur were cultured and isolated cancer cells were found to express RFP. (d) ARCaP_{M} cells in the metastatic tibial/femur tumor could also be seen in formaldehyde fixed sections, either by conventional H/E stain or directly by red florescence imaging. These analyses unanimously confirmed that the signals attained in IR-783 imaging reflect metastases of the orthotopic ARCaP_{M} tumor. (B) Detection of spontaneous prostate and intestine tumors in transgenic mouse models. (a) Whole body NIR fluorescent imaging of TRAMP mouse before dye injection, which revealed no background NIR fluorescence. (b) Whole body X-ray imaging of the animal. (c) Whole body NIR fluorescent imaging of TRAMP mouse revealed only tumor-positive signal after IR-783 i.p. injection at a dose of 10 nmol/20 g. (d) Fluorescence imaging picture of TRAMP mice merged with x-ray picture. (e) The prostate tumor dissected from this TRAMP mouse showed a strong NIR signal even after fixation in 4% formalin solution for 3 weeks. (f) The presence of tumor cells was confirmed by histopathology (H/E stain, 100×). (C) Detection of multiple intestinal neoplasia in Apc^{Min/+} mice after the administration of IR-783 i.p at a dose of 10 nmol/20 g with the Olympus OV110 imaging system. (a) Bright field photograph of a dissected intestine in the imaging chamber. (b) NIR heptamethine cyanine dye imaging of multiple tumors along the intestine, with two tumor nodules indicated with white arrows. (c and d) These two nodules were excised and adenoma was confirmed in these specimens by H/E staining (100×).

FIG. 42 depicts distribution of heptamethine cyanine dye IR-783 and its metabolites in tissues; time-course and concentration-dependent studies in normal and tumor-bearing mice in accordance with various embodiments of the present invention. Panels A, B, C and D are described below. (A) Normal organs dissected at 0, 6 and 80 hrs after IR-783 i.v. injection at a dose of 10 nmol/20 g were subjected to NIR dye imaging with a Kodak Imaging Station 4000 MM. Note that at 80 hrs, IR-783 was completely cleared from all vital organs examined. (B) A representative mouse bearing orthotopic ARCaP_{M} human prostate tumor was imaged after IR-783 10 nmol/20 g i.v. injection at 0.5, 24, 48, 72 and 96 hrs. Note dye uptake and retention seen in an ARCaP_{M} orthotopic tumor. (C) A representative mouse bearing a subcutaneous ARCaP_{M} tumor subjected to NIR imaging after IR-783 i.p. injection at a dose of 10 nmol/20 g. The left panel shows the retention of IR-783 in the tumor 24 hrs after dye administration in whole body in vivo imaging. The right panel shows the ex vivo imaging of surgically dissected tissues which confirmed the uptake and retention of IR-783 in a surgically dissected ARCaP_{M} tumor. Top row of this panel from left: liver, lung, and heart. Bottom row of this panel from left: spleen, kidneys, and tumor. Tumor tissue displayed strong signals in both in vivo and ex vivo imaging. (D) A standard curve was constructed based on the fluorescence emission intensity of IR-783 at 820 nm with the dye added to a PBS solution at concentrations of 0.5, 1, 2, 4, 8, 16 and 32 μM. The correlation coefficient between the fluorescence emission intensity and concentration of IR-783 was estimated to be r=0.9991 (see left panel). The apparent dye concentration (μg/g) in organs and tumor was calculated based on the standard curve established above (see right panel). The apparent dye concentration is defined here by the light emission intensity at 820 nm, which could include the parental IR-783 and its metabolites. Data are expressed as average±SEM of 3 determinations.

FIG. 43 depicts detection of human prostate cancer cells in human blood in accordance with various embodiments of the present invention. Panels A and B are described below. (A) ARCaP_{M} cells mixed with human blood were incubated with IR-783 and the particulate fractions containing normal healthy mononuclear cells and cancer cells were isolated using gradient centrifugation. The cells were resuspended in PBS for acquisition of fluorescent images under a confocal microscope. Significant uptake and retention of the dye could be detected in ARCaP_{M} cells in a fluorescent field (white arrow), while mononuclear cells hardly showed any signals (black arrow). (B) To determine the sensitivity of this novel method for tumor cell detection, known numbers of ARCaP_{M} cells (10 to 1,000 cells) were added to 1 ml of whole blood. Following gradient centrifugation, washing and re-suspension, positive fluorescent cancer cells were counted. Results presented in the histograph represent three separate experiments (n=3) with data expressed as mean+/−SEM.

FIG. 44 depicts correlation of NIR fluorescence intensity and concentration of cyanine dye in cancer cells in accordance with various embodiments of the present invention. Prostate cancer cells (ARCaP_{M}) were plated on live-cell imaging chambers and imaged 30 min after adding IR-783 at the following concentrations: 1 μM, 10 μM, 20 μM and 50 μM using a Perkin-Elmer disc confocal microscope as described. Images were acquired at 650 nm emission using a 63× objective. The mean pixel intensity of IR-783 in cells was quantitated using Metamorph 6.1 software. Panels A and B are described below. (A) Images of IR-783 dye uptake in ARCaP_{M} cells at concentration of 1 μM, 10 μM, 20 μM and 50 μM. (B) The fluorescence emission intensity was measured and correlated with the concentrations of IR-783 in ARCaP_{M} cells (r=0.997).

FIG. 45 depicts structural requirement of heptamethine cyanine dyes for cancer cell uptake and retention in accordance with various embodiments of the present invention. A series of heptamethine cyanine dyes and their derivatives were screened in cultured MIA PaCa-2 human pancreatic cancer cells with parental MHI-148 served as a positive control. Results showed that IR-1, IR-2, IR-3 (modifications of MHI-148) and IR-4 (4-aminothiophenol derivative of IR-783) were found to be devoid of any uptake and retention by this cancer cell line when compared with MHI-148.

FIG. 46 depicts structural requirement of the uptake and retention of heptamethine cyanine dyes by tumor tissues in mice in accordance with various embodiments of the present invention. Mice bearing human renal cancer SN12C at subcutaneous sites were injected i.p. with MHI-148, IR-1, IR-2, IR-3 and IR-4 heptamethine cyanine dyes at a dose of 10 nmol per mouse. Whole-body optical imaging was taken at 24 hrs using a Kodak Imaging System. Strong signals were visualized in tumors after MHI-148 injection (see white arrows) while background fluorescence signals were observed in mice injected with IR-1, IR-2, IR-3 or IR-4 (see black arrows).

FIG. 47 depicts the uptake and retention of IR-783 dye by human and mouse cancer cells in accordance with various embodiments of the present invention. Human bladder cancer cell (T-24), renal cancer cell (ACHN), and mouse pancreatic cancer cell lines (PDAC2.3, PDAC3.3, BTC3 and BTC4) showed significant uptake after incubating with 20 μM IR-783 dye. The images show IR-783 staining (NIR), cell morphology (BF) and a merger of the two images (Merge). All the images were acquired at 400× magnification.

FIG. 48 depicts significant uptake of IR-783 observed in malignant cells in accordance with various embodiments of the present invention. Dyes were not taken up by non-cancerous human embryonic fetal kidney cells (HEK293). Panels A and B are described below. (A). The overlay of the NIR imaging with Mito tracker imaging and Lyso tracker imaging shows nearly exact concordance in staining as evidenced by the purple to red and green colors seen in (B).

FIG. 49 depicts the detection of higher signals in tumor specimens in ex vivo analysis; other normal organs displayed very low signals. Panels A and B are described below. (A) in accordance with various embodiments of the present invention. The further NIR imaging results showed that both subrenal capsule renal tumor (Caki-1) and intraosseous renal tumor (SN12C) xenografts in mice displayed strong signal at the anatomical sites where tumors were implanted (B).

FIG. 50 depicts detection in human kidney tumor and normal tissues excised from the clinical samples after nephrectomy in accordance with various embodiments of the present invention. Compared with another heptamethine dye IR-780 and PBS, IR-783 can be observed showing stronger signals in tumor tissues than the IR-780 and PBS group. Panels A, C, D, and E are described below. In samples containing normal and tumor areas, only tumor cells can take up IR-783, even the normal tissues next to the tumor cannot retain the IR-783 dyes (A and C). The frozen tissue confocal NIR imaging confirmed that the uptake in the normal kidney tissues were undetectable while significant uptake were found in tumor tissues (D and E).

FIG. 51 shows, see panel A, that cancer cells can be clearly visualized after dye mixing with human blood, but without dye staining, the cancer cells can not be identified from normal mononuclear cells under NIR imaging. Panel B with subpanels α-d is described below. The flow cytometry results showed that the cancer cells staining IR-783 were totally identified from normal lymphocytes (see Q2 in panel B-a and panel B-b). Unstained samples displayed that there were no difference of dye distribution between cancer cells and lymphocytes (see panel B-c and panel B-d).

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

Carbocyanine dyes represent a group of NIR organic dyes which emit fluorescence light, with high extinction coefficient, and can allow the visualization of cancer cells or solid tumors even when a small number of dye molecules are taken up into the cells or tissues. NIR fluorescent light is defined as a wide-region of the electromagnetic spectra from 680-1600 nm. Because NIR organic dyes with light emission at NIR wavelength range will be minimally interfered by the autofluorescence of the hot tissues, NIR compounds are therefore recognized as having attractive properties for the detection of tumors in mice or humans with minimal background activity. Two of the organic dyes, IR-780 and IR-783, the inventors have utilized in their study as described herein are commercially available from Sigma-Aldrich, whereas one of the organic dyes MHI-148, used and described herein is chemically synthesized in the laboratory. In addition to the use of these two organic dyes for molecular imaging and targeting of tumor cells, additional organic dyes with similar chemical structures will have similar imaging and targeting properties for cancer cells and are included within the scope of the present invention.

Indocyanine green dyes have proved to be useful for measuring blood flow and cardiac output, as well as imaging tumors (2, 3, 37). The chemical structures of water-soluble pentamethine and heptamethine cyanine dyes have recently been modified to increase their chemical stability, photostability, and quantum yield (1). IR-780, IR-783 and MHI-148 are such new dyes, modified with a rigid cyclohexenyl substitution in the polymethine linker. These NIR dyes can be actively taken up and accumulated by cancer cells but not by normal cells. The salient features of these dual imaging and targeting NIR dyes, which are covered by various embodiments of present invention, are: (1) Detecting cancer cells and cancer metastases by directly uptaking the dyes into cancer and not normal cells without the requirement of chemical conjugation. (2) Detecting many other tumor types and tumor cell populations under cell culture and in vivo conditions. The cancer-specific uptake and retention of these dyes is likely to be mediated by OATPs since the transport of these dyes into cancer cells can be antagonized by BSP, an OATP competitive inhibitor (38, 39). (3) Serving as potential carriers for drug payloads or radioactive agents to increase the specificity and reduce the toxicity of therapeutic agents by preferential uptake and accumulation in cancer cells but not in normal cells. This also allows for non-invasive detection and assessment of the concentrations of the drug payload or radioactive agents in tumors by simply monitoring the NIR fluorescence associated with tumor tissues in live subjects.

The cyanine dyes are water soluble, so they have rapid clearance and are unlikely to be trapped in the reticular endothelium of the liver, lung or spleen. They were found to be superior for cancer detection to other cyanine dyes such as indocyanine green and non-cyanine dyes such as rhodamine 123 (data not shown). Imaging with NIR dyes can yield much higher signal/noise ratios with minimal interfering background fluorescence. The fluorescence efficiency of cyanine dyes can increase by ˜1,000-fold upon binding to proteins and nucleic acids (36). The stable binding, together with the shift toward increased fluorescence could be highly beneficial, accounting for the “trapping” of the NIR signals in cancer cells for prolonged periods (>5 days) and allowing tumor detection in live animals with high signal/noise ratios. The stability of these cyanine dyes after formalin fixation allows for new and sensitive methods of detecting cancer cells in whole blood and in harvested surgical specimens by injecting the cyanine dyes prior to sampling at the time of surgery. In practice, these could help physicians and pathologists follow up patients with possible circulating cancer cells in blood and assess surgical margins at the time of surgery. Described herein, the differential dye uptake and retention by cancer and normal cells and tissues can be demonstrated robustly by the use of a variety of detecting devices including Zeiss LSM 510 META, Kodak 4000 MM, and Olympus OV100 systems. These different detecting methodologies were adopted based on their sensitivity and capability of allowing merging of images obtained via different detection modalities (e.g. X-ray and NIR imagings). The wide range of detecting devices used in this study supports the conclusion that IR-780, IR-783 and MHI-148 are preferentially taken up and retained by cancer but not normal cells.

The mechanisms by which these cyanine dyes cross the cytoplasmic membranes of cancer cells but not normal cells were investigated. It was concluded that the uptake was mediated by proteins of the OATP family, because the active uptake could be effectively blocked by BSP, a known OATP competitive inhibitor. OATPs are well-recognized as channels for the transport of a diverse group of substrates including bile acids, hormones, xenobiotics and their metabolites (40-42). Results from this study are consistent with published reports which indicate differences in the type and levels of OATPs between cancer and normal cells (43-46). Moreover certain members of OATPs have recently been shown to be overexpressed in various human cancer tissues as well as in cancer cell lines (47-50) and the confirmation of OATPs as the key mediator of heptamethine cyanine dye uptake and retention in tumor cells warrants further investigation.

The ability of mouse tumors to accumulate these cyanine dyes is of great significance. This will facilitate the use of these dyes in immune-intact syngenic and transgenic mouse models to study the fundamentals of cancer biology, metastasis and therapy. Since these dyes can be further explored as generalized ligands for all malignant cells, the synthesis of dye-antineoplastic drug conjugates, dye-radiolabeled drug conjugates and dye-toxin conjugates could immensely facilitate the development of new therapeutics to treat cancer and pre-cancerous conditions.

The heptamethine cyanine dye (IR-783) is a water-soluble heptamethine cyanine dye and its stability and fluorescence efficiency in aqueous media, as well as specificity to tumor cells are beneficial to cancer detection. The molecule is composed of two polycyclic parts (benzoindotricarbocyanin), which are quite lipophilic and are linked by a carbon chain. A sulfate group is bound to each polycyclic part, leading to some water solubility. The absorption spectrum of IR-783 exhibits a strong band between 600 and 900 nm (maximum absorption is 782 nm in aqueous media).⁶⁶

Various embodiments of the present invention provides for the use of IR-783 as a sensitive tracer molecule for renal cancer targeting and an ideal imaging agent for renal cancer detection. The in vitro and in vivo application of IR-783 in near infrared imaging are described herein and specific uptake of IR-783 in renal cancer cells but not normal cells and long-lasting accumulating of this indocyanine dye in mouse tumor xenografts and human renal tumor tissues are shown herein. Being stained with IR-783, renal cancer cells can be identified from normal cells in blood. The dual imaging and targeting property of this heptamethine cyanine dye could be further exploited for improved modalities of cancer detection, diagnosis and therapy.

Described herein, heptamethine cyanine dyes (IR-783) displayed its imaging and targeting capabilities in human renal cancer. There are several important features to this cyanine dye and this detection approach. (1) Both in vitro and in vivo results can provide accurate information that this cyanine dye can be specifically taken up in not only culture cancer cells but also animal tumor xenografts and human tumor samples. These water-soluble cyanine dyes can retain in tumors for prolonged periods (>5 days) and detect tumors in live animals with high signal/noise ratios. (2) These imaging and targeting dyes can be used to detect cancer cells directly without the requirement of chemical conjugation. The classical NIR polymethine fluorophores, however, mostly lack tumor-specificity and hence require chemical conjugation prior to application for cancer imaging. The disadvantage of the prior art dye-conjugating imaging is that the detection only be limited on specific cancer cell types and can not be applied widely on other tumors.^{64, 65} (3) The fluorescence of this heptamethine cyanine dye can be readily detected from deep tissues such as subrenal capsule and intraosseous tumor xenografts, unlike other NIR dyes with poor fluorescence efficiency.

The uptake of this cyanine dye was mediated by organic anion transporting peptides (OATPs), because the active uptake can be blocked by bromosulfophthalein (BSP), a competitive inhibitor of OATPs.⁷³ The over-expression of some OATPs in prostate cancer and renal cancer was also found. These results are consistent with published reports which indicate differences in the type and levels of OATPs between cancer and normal cells.⁷⁴⁻⁷⁷

Over the past 2-3 decades, the incidence of kidney cancer has steadily increased in the United States. A great proportion of the newly small (<4 cm), low-stage diagnosed renal cortical tumors are more subjected to partial nephrectomy.⁷⁸ The application of this cyanine dye can give surgeons more opportunities to identify the cut-off area and the negative surgical margin (NSM) during partial nephrectomy. Because of the vascular dissemination of renal cancer carcinoma, the inventors designed a detection study for RCC cells in blood using the IR-783 cyanine dye and the results confirm that cancer cells can be differentiated from normal mononuclear cells in blood. Compared with some microfluidic platform technique by utilizing the stiffer and larger size characteristic of cancer cells, the application of this cyanine dye improves the sensitivity of detection⁷⁹. With the improvement of the sensitivity of this method, IR-783 can be further used in renal tumor early-stage diagnosis and clinical monitoring of the renal cancer after therapy. Moreover, it is possible to use this type of technique to isolate the individual cancer cells from patients and conduct a complete genome and gene expression analyses which could increase the capabilities of predicting the progression of human cancers.

In sum, heptamethine cyanine dyes were demonstrated to selectively target cancer but not normal cells, irrespective of their species and organ of origin. Application of NIR fluorescent dyes in the clinic allows for the management of cancer patients on an individual basis. Further, a heptamethine cyanine dye was demonstrated to be taken up in renal cancer cells but not normal cells and to be an ideal targeting and imaging agent for renal cancer detection. Future application of NIR fluorescence dyes in the clinic could make important progress on the early diagnosis and follow-up management of renal cancer patients.

Accordingly, various embodiments of the present invention provides for identifying, detecting, imaging, isolating, characterizing and locating a cancer cell or a tumor in a subject.

Embodiments of the present invention can improve patient care and offer personalized oncology. For example, these methods can assist surgeons during operation to identify tumors in surgical specimens, to recognize the residual cancer cells at the surgical margin, and to recognize tumor locations before, during and after surgery. These methods can assist medical and radiation oncologists to identify the location of the tumors and their metastases, to determine tumor shrinkage during the course of treatment and to determine the pharmacokinetics and pharmacodynamics of the drugs or treatment agents in the tumor. These methods can also assist pathologists and laboratory technologists to recognize the quantity and location of a tumor cell in bodily fluids, secreted gene products in bodily fluids and therapeutic responses during the course and follow-up of the patients.

Near-Infrared (NIR) Organic Carbocyanine Dyes

Various embodiments of the present invention uses various near-infrared (NIR) organic carbocyanine dyes, which are described herein. In various embodiments, the NIR organic carbocyanine dyes are those described by International Patent Application Publication No. WO 2009012109, herein incorporated by reference as though fully set forth in its entirety and are briefly described below.

In various embodiments, the NIR organic carbocyanine dye used in the methods of the present invention is a compound having two cyanine ring (“CyR”) structures as defined below, linked by an optionally substituted linker as shown below:

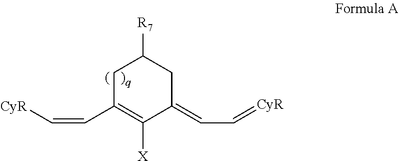

where X is selected from the group consisting of: hydrogen, halogen, CN, Me, phenyl, OH, OMe, OPh, 4-O-Ph-NH₂, 4-O-Ph-CH₂CH₂COOH, 4-O-Ph-CH₂CH₂CONHS (where NHS is a group derived from N-hydroxysuccinimide or succinimide-N-oxy), NH-Ph, NHEt, SEt, S-Ph, 4-S-Ph-COOH, 4-S-Ph-OH, 4-O-Ph-COOH, 4-O-Ph-NCS, and 4-S-Ph-NCS; q is 0 (forming a cyclopentene ring) or 1 (forming a cyclohexene ring); R₇ is selected from H and COOR⁹, where R⁹ is H, CH₃, or CH₂CH₃. In one embodiment, when q is 0, R7 is H.

In various embodiments, the CyR structures include those shown below. The CyR structures are generally heterocyclic end units comprising cyanine. The line shown on the ring structures below shows where the linker is attached. Each portion of the molecule may have various substituents.

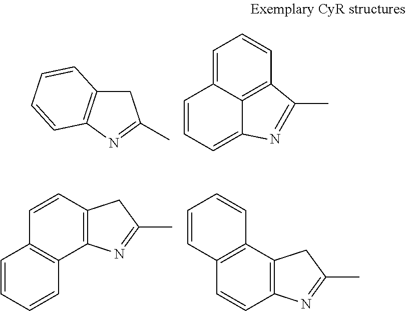

In various embodiments, heptamethine cyanine dyes (having 7 carbons between CyR structures) are used in the methods of the present invention.

It is understood that one or both nitrogen atoms in the cyanine ring structures may have a positive charge, in which case a suitable counterion is associated with the compound. The CyR structures may be substituted with any suitable substituent on any suitable ring position, for example, as shown below:

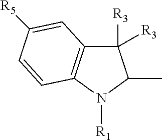

where R5 is selected from H, OH, OMe, halogen, NH₂, NHR, NR₂ and COOH, where each R is independently C1-C6 alkyl and R₁ and R₃ are as described herein.

Other cyanine ring structures include:

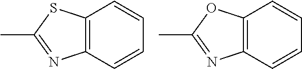

In various embodiments, the NIR organic carbocyanine dyes used in the methods of the present invention are compounds having formula B:

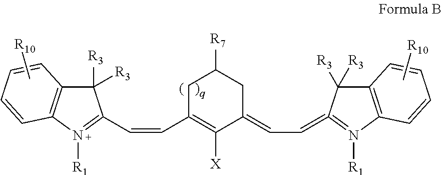

where X is selected from the group consisting of: hydrogen, halogen, CN, Me, phenyl, OH, OMe, OPh, 4-O-Ph-NH₂, 4-O-Ph-CH₂CH₂COOH, 4-O-Ph-CH₂CH₂CONHS, NH-Ph, NHEt, SEt, S-Ph, 4-S-Ph-COOH, 4-S-Ph-OH, 4-O-Ph-COOH, 4-O-Ph-NCS, and 4-S-Ph-NCS; q is 0 or 1; R7 is selected from H and COOR⁹, where R⁹ is H, CH₃, or CH₂CH₃; each R¹ is independently in each instance, (CH₂)ₘR_{A}, where m is an integer from 1 to 12, R_{A} is independently CH₃, NH₂, SH, COOH, SO₃H, OH, halogen and CO—N-hydroxysuccinimide; each Rio is independently in each instance selected from H, OH, OMe, halogen, NH₂, NHR, NR₂ and COOH, where each R is independently C1-C6 alkyl; each R₃ is independently in each instance, selected from the group consisting of: methyl and phenyl. Rio may be independently attached to any available position on each ring.

In various embodiments, X is Cl and each R₃ is CH₃. In an embodiment, R¹ is (CH₂)ₘCOOH and m is 1-6. In an embodiment, R¹ is (CH₂)ₘSO₃H and m is 1-6. In an embodiment, R₁ is (CH₂)ₘCH₃ and m is 1-6. In various embodiments, IR organic carbocyanine dye used in the methods of the present invention is MHI-148, IR-783, or IR-780. In various embodiments, the NIR organic carbocyanine dye used in the methods of the present invention is a compound of Formula A. In various embodiments, the NIR organic carbocyanine dye used in the methods of the present invention is a compound of Formula B.

Specific embodiments of the NIR organic carbocyanine dye used in the methods of the present invention are provided in Formula C,

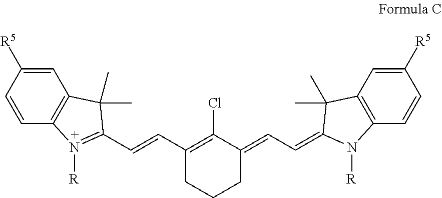

wherein each R⁵ is independently selected from the group consisting of: H, OH, OMe, halogen, NH₂, NHR^{B}, NR^{B}₂, COOH, where each R^{B} is independently C1-C3 alkyl and R is as provided below.

Particular exemplary NIR organic carbocyanine dyes are shown below:

| Compound Number | Groups in Formula C |
|---|---|
| MHI-148 | R═(CH₂)₅COOH, R⁵═H |
| MHI-25 (aka IR-783) | R═(CH₂)₄SO₃H, R⁵═H |
| MHI-78 | R═(CH₂)₂OH, R⁵═H |
| MHI-160 | R═(CH₂)₄COOH |
| MHI-161 | R═(CH₂)₃COOH |
| MHI-200 | R═(CH₂)ₙCOOH |
| | n = 2-4, 7-10, 12 |
| IR-780 | R═(CH₂)₂CH₃ |

In various embodiments of the invention, the R substituent groups on the cyanine ring groups are not the same. In embodiments of the invention, the R substituent groups on the cyanine ring groups are the same. Certain embodiments of the invention contain two acid R groups. Certain embodiments of the invention contain one acid and one ester R group. Certain embodiments of the invention contain two ester R groups.

In various embodiments of the invention, cyanine-containing compounds according to any of Formulas (I), (II), (III), (IV), (V), (VI), (VII), or (VIII) are used in the methods of the present invention.

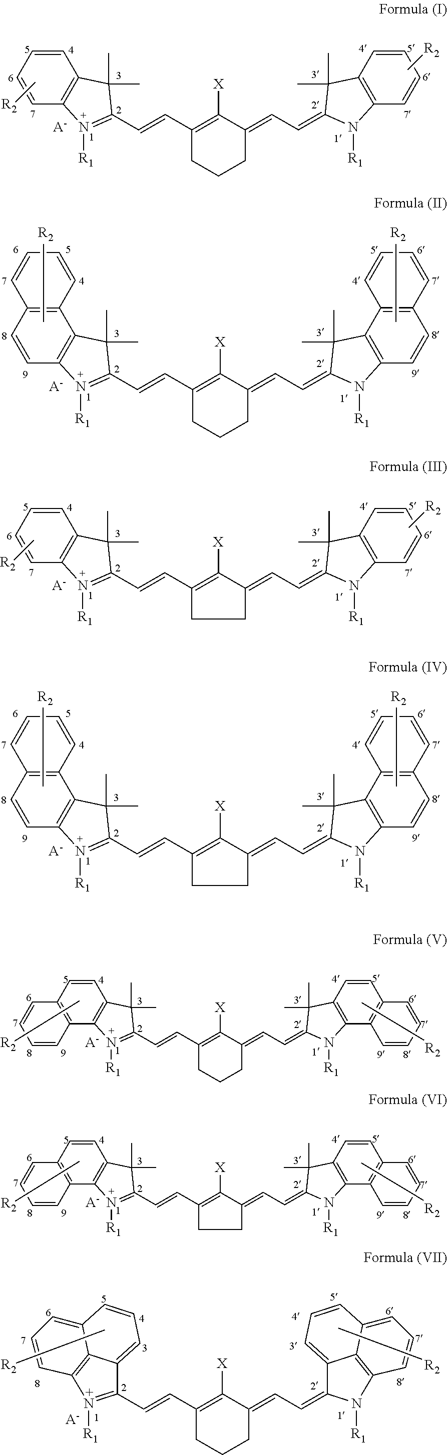

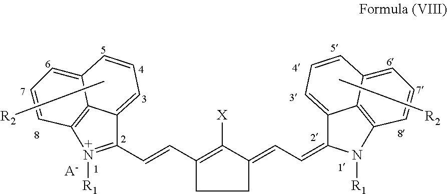

wherein: each R₂ is independently in each instance selected from the group consisting of hydrogen, any electron withdrawing group (EWG) and any electron donating group (EDG) attached at one or more of positions 3, 3′, 4, 4′, 5, 5′, 6, 6′, 7, 7′, 8, 8′; each R₁ is independently in each instance selected from the group consisting of: hydrogen, alkyl, aryl, aralkyl, alkylsulfonato, alkylcarboxylic, alkylamino; X is chlorine or bromine; and counteranion A is selected from the group consisting of: iodide, bromide, arylsulfonato, alkylsulfonato, tetrafluoroborate; chloride and any other pharmaceutically acceptable anions. Electron donating and withdrawing groups are known in the art. Some examples of electron donating groups include: OH, OMe, NH₂, NHR^{B}, and NR^{B}₂, where R^{B} is C1-C6 alkyl. Some examples of electron withdrawing groups include: halogen, COOH, CN, SO₃Na, COOH, COOMe, and COOEt.

Some specific embodiments of compounds used in the methods of the present invention are listed below.

Formula 1

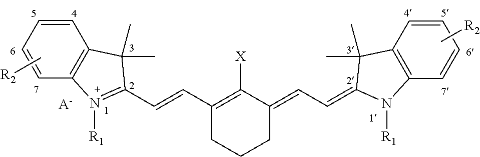

| X | R₁ | R₂*** |
|---|---|---|
| Br | Methyl | H, EDG, EWG |
| Br | Ethyl | H, EDG, EWG |
| Br | Propyl | H, EDG, EWG |
| Br | Butyl* | H, EDG, EWG |
| Br | Pentyl* | H, EDG, EWG |
| Br | Hexyl* | H, EDG, EWG |
| Br | Heptyl* | H, EDG, EWG |
| Br | Octyl* | H, EDG, EWG |
| Br | Nonyl* | H, EDG, EWG |
| Br | Decyl* | H, EDG, EWG |
| Br | Undecyl* | H, EDG, EWG |
| Br | Dodecyl* | H, EDG, EWG |
| Br | Tridecyl* | H, EDG, EWG |
| Br | Tetradecyl* | H, EDG, EWG |
| Br | Pentadecyl* | H, EDG, EWG |
| Br | Hexadecyl* | H, EDG, EWG |
| Br | Heptadecyl* | H, EDG, EWG |
| Br | Octadecyl* | H, EDG, EWG |
| Br | Phenyl** | H, EDG, EWG |
| Br | Benzyl** | H, EDG, EWG |
| Br | Napthyl** | H, EDG, EWG |
| Br | CH₂—SO₃⁻ | H, EDG, EWG |
| Br | (CH₂)₂—SO₃⁻ | H, EDG, EWG |
| Br | (CH₂)₃—SO₃⁻ | H, EDG, EWG |
| Br | (CH₂)₄—SO₃⁻ | H, EDG, EWG |
| Br | (CH₂)₅—SO₃⁻ | H, EDG, EWG |
| Br | (CH₂)₆—SO₃⁻ | H, EDG, EWG |
| Br | (CH₂)₇—SO₃⁻ | H, EDG, EWG |
| Br | (CH₂)₈—SO₃⁻ | H, EDG, EWG |
| Br | (CH₂)₉—SO₃⁻ | H, EDG, EWG |
| Br | (CH₂)₁₀—SO₃⁻ | H, EDG, EWG |
| Br | (CH₂)₁₁—SO₃⁻ | H, EDG, EWG |
| Br | (CH₂)₁₂—SO₃⁻ | H, EDG, EWG |
| Br | (CH₂)₁₃—SO₃⁻ | H, EDG, EWG |
| Br | (CH₂)₁₄—SO₃⁻ | H, EDG, EWG |
| Br | (CH₂)₁₅—SO₃⁻ | H, EDG, EWG |
| Br | (CH₂)₁₆—SO₃⁻ | H, EDG, EWG |
| Br | (CH₂)₁₇—SO₃⁻ | H, EDG, EWG |
| Br | (CH₂)₁₈—SO₃⁻ | H, EDG, EWG |
| Br | CH₂—CO₂⁻ | H, EDG, EWG |
| Br | (CH₂)₂—CO₂⁻ | H, EDG, EWG |
| Br | (CH₂)₃—CO₂⁻ | H, EDG, EWG |
| Br | (CH₂)₄—SO₂⁻ | H, EDG, EWG |
| Br | (CH₂)₅—CO₂⁻ | H, EDG, EWG |
| Br | (CH₂)₆—CO₂⁻ | H, EDG, EWG |
| Br | (CH₂)₇—CO₂⁻ | H, EDG, EWG |
| Br | (CH₂)₈—CO₂⁻ | H, EDG, EWG |
| Br | (CH₂)₉—CO₂⁻ | H, EDG, EWG |
| Br | (CH₂)₁₀—CO₂⁻ | H, EDG, EWG |
| Br | (CH₂)₁₁—CO₂⁻ | H, EDG, EWG |
| Br | (CH₂)₁₂—CO₂⁻ | H, EDG, EWG |
| Br | (CH₂)₁₃—CO₂⁻ | H, EDG, EWG |
| Br | (CH₂)₁₄—CO₂⁻ | H, EDG, EWG |
| Br | (CH₂)₁₅—CO₂⁻ | H, EDG, EWG |
| Br | (CH₂)₁₆—CO₂⁻ | H, EDG, EWG |
| Br | (CH₂)₁₇—CO₂⁻ | H, EDG, EWG |
| Br | (CH₂)₁₈—CO₂⁻ | H, EDG, EWG |
| Br | CH₂—NH₂ | H, EDG, EWG |
| Br | (CH₂)₂—NH₂ | H, EDG, EWG |
| Br | (CH₂)₃—NH₂ | H, EDG, EWG |
| Br | (CH₂)₄—NH₂ | H, EDG, EWG |
| Br | (CH₂)₅—NH₂ | H, EDG, EWG |
| Br | (CH₂)₆—NH₂ | H, EDG, EWG |
| Br | (CH₂)₇—NH₂ | H, EDG, EWG |
| Br | (CH₂)₈—NH₂ | H, EDG, EWG |
| Br | (CH₂)₉—NH₂ | H, EDG, EWG |
| Br | (CH₂)₁₀—NH₂ | H, EDG, EWG |
| Br | (CH₂)₁₁—NH₂ | H, EDG, EWG |
| Br | (CH₂)₁₂—NH₂ | H, EDG, EWG |
| Br | (CH₂)₁₃—NH₂ | H, EDG, EWG |
| Br | (CH₂)₁₄—NH₂ | H, EDG, EWG |
| Br | (CH₂)₁₅—NH₂ | H, EDG, EWG |
| Br | (CH₂)₁₆—NH₂ | H, EDG, EWG |
| Br | (CH₂)₁₇—NH₂ | H, EDG, EWG |
| Br | (CH₂)₁₈—NH₂ | H, EDG, EWG |

*Each alkyl chain is optionally branched with an alkyl chain, aryl ring, heteroaryl, aralkyl group, or unsaturation at any position on the chain.
**The phenyl, benzyl, or naphthyl ring is optionally ortho-, meta-, or para-substituted with 1-3 substituents selected from halo, alkoxy, hydroxyl, CF₃, NO₂, NH₂, NHR, or NR₂, where R is H or C1-C3 alkyl.
***The R₂ group is H, any electron withdrawing group, or any electron donating group.

The A group is I, Cl, Br, OSO₂R, BF₄, ClO₄, or any pharmaceutically acceptable anion. In embodiments of compounds of Formulas 1-8, X can be Cl or Br, for example.

Formula 2

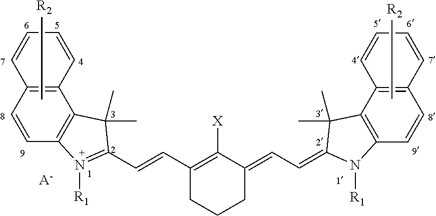

| X | R₁ | R₂*** |
|---|---|---|
| Br | Methyl | H, EDG, EWG |
| Br | Ethyl | H, EDG, EWG |
| Br | Propyl | H, EDG, EWG |
| Br | Butyl* | H, EDG, EWG |
| Br | Pentyl* | H, EDG, EWG |
| Br | Hexyl* | H, EDG, EWG |
| Br | Heptyl* | H, EDG, EWG |
| Br | Octyl* | H, EDG, EWG |
| Br | Nonyl* | H, EDG, EWG |
| Br | Decyl* | H, EDG, EWG |
| Br | Undecyl* | H, EDG, EWG |
| Br | Dodecyl* | H, EDG, EWG |
| Br | Tridecyl* | H, EDG, EWG |
| Br | Tetradecyl* | H, EDG, EWG |
| Br | Pentadecyl* | H, EDG, EWG |
| Br | Hexadecyl* | H, EDG, EWG |
| Br | Heptadecyl* | H, EDG, EWG |
| Br | Octadecyl* | H, EDG, EWG |
| Br | Phenyl** | H, EDG, EWG |
| Br | Benzyl** | H, EDG, EWG |
| Br | Napthyl** | H, EDG, EWG |
| Br | CH₂—SO₃⁻ | H, EDG, EWG |
| Br | (CH₂)₂—SO₃⁻ | H, EDG, EWG |
| Br | (CH₂)₃—SO₃⁻ | H, EDG, EWG |
| Br | (CH₂)₄—SO₃⁻ | H, EDG, EWG |
| Br | (CH₂)₅—SO₃⁻ | H, EDG, EWG |
| Br | (CH₂)₆—SO₃⁻ | H, EDG, EWG |
| Br | (CH₂)₇—SO₃⁻ | H, EDG, EWG |
| Br | (CH₂)₈—SO₃⁻ | H, EDG, EWG |
| Br | (CH₂)₉—SO₃⁻ | H, EDG, EWG |
| Br | (CH₂)₁₀—SO₃⁻ | H, EDG, EWG |
| Br | (CH₂)₁₁—SO₃⁻ | H, EDG, EWG |
| Br | (CH₂)₁₂—SO₃⁻ | H, EDG, EWG |
| Br | (CH₂)₁₃—SO₃⁻ | H, EDG, EWG |
| Br | (CH₂)₁₄—SO₃⁻ | H, EDG, EWG |
| Br | (CH₂)₁₅—SO₃⁻ | H, EDG, EWG |
| Br | (CH₂)₁₆—SO₃⁻ | H, EDG, EWG |
| Br | (CH₂)₁₇—SO₃⁻ | H, EDG, EWG |
| Br | (CH₂)₁₈—SO₃⁻ | H, EDG, EWG |
| Br | CH₂—CO₂⁻ | H, EDG, EWG |
| Br | (CH₂)₂—CO₂⁻ | H, EDG, EWG |
| Br | (CH₂)₃—CO₂⁻ | H, EDG, EWG |
| Br | (CH₂)₄—CO₂⁻ | H, EDG, EWG |
| Br | (CH₂)₅—CO₂⁻ | H, EDG, EWG |
| Br | (CH₂)₆—CO₂⁻ | H, EDG, EWG |
| Br | (CH₂)₇—CO₂⁻ | H, EDG, EWG |
| Br | (CH₂)₈—CO₂⁻ | H, EDG, EWG |
| Br | (CH₂)₉—CO₂⁻ | H, EDG, EWG |
| Br | (CH₂)₁₀—CO₂⁻ | H, EDG, EWG |
| Br | (CH₂)₁₁—CO₂⁻ | H, EDG, EWG |
| Br | (CH₂)₁₂—CO₂⁻ | H, EDG, EWG |
| Br | (CH₂)₁₃—CO₂⁻ | H, EDG, EWG |
| Br | (CH₂)₁₄—CO₂⁻ | H, EDG, EWG |
| Br | (CH₂)₁₅—CO₂⁻ | H, EDG, EWG |
| Br | (CH₂)₁₆—CO₂⁻ | H, EDG, EWG |
| Br | (CH₂)₁₇—CO₂⁻ | H, EDG, EWG |
| Br | (CH₂)₁₈—CO₂⁻ | H, EDG, EWG |
| Br | CH₂—NH₂ | H, EDG, EWG |
| Br | (CH₂)₂—NH₂ | H, EDG, EWG |
| Br | (CH₂)₃—NH₂ | H, EDG, EWG |
| Br | (CH₂)₄—NH₂ | H, EDG, EWG |
| Br | (CH₂)₅—NH₂ | H, EDG, EWG |
| Br | (CH₂)₆—NH₂ | H, EDG, EWG |
| Br | (CH₂)₇—NH₂ | H, EDG, EWG |
| Br | (CH₂)₈—NH₂ | H, EDG, EWG |
| Br | (CH₂)₉—NH₂ | H, EDG, EWG |
| Br | (CH₂)₁₀—NH₂ | H, EDG, EWG |
| Br | (CH₂)₁₁—NH₂ | H, EDG, EWG |
| Br | (CH₂)₁₂—NH₂ | H, EDG, EWG |
| Br | (CH₂)₁₃—NH₂ | H, EDG, EWG |
| Br | (CH₂)₁₄—NH₂ | H, EDG, EWG |
| Br | (CH₂)₁₅—NH₂ | H, EDG, EWG |
| Br | (CH₂)₁₆—NH₂ | H, EDG, EWG |
| Br | (CH₂)₁₇—NH₂ | H, EDG, EWG |
| Br | (CH₂)₁₈—NH₂ | H, EDG, EWG |

*Each alkyl chain is optionally branched with an alkyl chain, aryl ring, heteroaryl, aralkyl group, or unsaturation at any position on the chain.
**The phenyl, benzyl, or naphthyl ring is optionally ortho-, meta-, or para-substituted with 1-3 substituents selected from halo, alkoxy, hydroxyl, CF₃, NO₂, NH₂, NHR, or NR₂.
***The R₂ group is H, any electron withdrawing group, or any electron donating group.

The A group is I, Cl, Br, OSO₂R, BF₄, ClO₄, or any pharmaceutically acceptable anions.

In various embodiments of compounds of Formula 2, X═Br, R₁=Me, and A=ClO₄.

In various embodiments of compounds of Formula 2, when compounds are used in methods of the present invention, compounds are not included where: X═Br, R₁=Me, and A=ClO₄.

Formula 3:

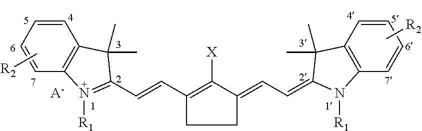

| X | R₁ | R₂*** |
|---|---|---|
| Br | Methyl | H, EDG, EWG |
| Br | Ethyl | H, EDG, EWG |
| Cl, Br | Propyl | H, EDG, EWG |
| Cl, Br | Butyl* | H, EDG, EWG |
| Cl, Br | Pentyl* | H, EDG, EWG |
| Cl, Br | Hexyl* | H, EDG, EWG |
| Cl, Br | Heptyl* | H, EDG, EWG |
| Cl, Br | Octyl* | H, EDG, EWG |
| Cl, Br | Nonyl* | H, EDG, EWG |
| Cl, Br | Decyl* | H, EDG, EWG |
| Cl, Br | Undecyl* | H, EDG, EWG |
| Cl, Br | Dodecyl* | H, EDG, EWG |
| Cl, Br | Tridecyl* | H, EDG, EWG |
| Cl, Br | Tetradecyl* | H, EDG, EWG |
| Cl, Br | Pentadecyl* | H, EDG, EWG |
| Cl, Br | Hexadecyl* | H, EDG, EWG |
| Cl, Br | Heptadecyl* | H, EDG, EWG |
| Cl, Br | Octadecyl* | H, EDG, EWG |
| Cl, Br | Phenyl** | H, EDG, EWG |
| Cl, Br | Benzyl** | H, EDG, EWG |
| Cl, Br | Naphthyl** | H, EDG, EWG |
| Cl, Br | CH₂—SO₃⁻ | H, EDG, EWG |
| Cl, Br | (CH₂)₂—SO₃⁻ | H, EDG, EWG |
| Cl, Br | (CH₂)₃—SO₃⁻ | H, EDG, EWG |
| Cl, Br | (CH₂)₄—SO₃⁻ | H, EDG, EWG |
| Cl, Br | (CH₂)₅—SO₃⁻ | H, EDG, EWG |
| Cl, Br | (CH₂)₆—SO₃⁻ | H, EDG, EWG |
| Cl, Br | (CH₂)₇—SO₃⁻ | H, EDG, EWG |
| Cl, Br | (CH₂)₈—SO₃⁻ | H, EDG, EWG |
| Cl, Br | (CH₂)₉—SO₃⁻ | H, EDG, EWG |
| Cl, Br | (CH₂)₁₀—SO₃⁻ | H, EDG, EWG |
| Cl, Br | (CH₂)₁₁—SO₃⁻ | H, EDG, EWG |
| Cl, Br | (CH₂)₁₂—SO₃⁻ | H, EDG, EWG |
| Cl, Br | (CH₂)₁₃—SO₃⁻ | H, EDG, EWG |
| Cl, Br | (CH₂)₁₄—SO₃⁻ | H, EDG, EWG |
| Cl, Br | (CH₂)₁₅—SO₃⁻ | H, EDG, EWG |
| Cl, Br | (CH₂)₁₆—SO₃⁻ | H, EDG, EWG |
| Cl, Br | (CH₂)₁₇—SO₃⁻ | H, EDG, EWG |
| Cl, Br | (CH₂)₁₈—SO₃⁻ | H, EDG, EWG |
| Cl, Br | CH₂—CO₂ | H, EDG, EWG |
| Cl, Br | (CH₂)₂—CO₂ | H, EDG, EWG |
| Cl, Br | (CH₂)₃—CO₂ | H, EDG, EWG |
| Cl, Br | (CH₂)₄—CO₂ | H, EDG, EWG |
| Cl, Br | (CH₂)₅—CO₂ | H, EDG, EWG |
| Cl, Br | (CH₂)₆—CO₂ | H, EDG, EWG |
| Cl, Br | (CH₂)₇—CO₂ | H, EDG, EWG |
| Cl, Br | (CH₂)₈—CO₂ | H, EDG, EWG |
| Cl, Br | (CH₂)₉—CO₂ | H, EDG, EWG |
| Cl, Br | (CH₂)₁₀—CO₂ | H, EDG, EWG |
| Cl, Br | (CH₂)₁₁—CO₂ | H, EDG, EWG |
| Cl, Br | (CH₂)₁₂—CO₂ | H, EDG, EWG |
| Cl, Br | (CH₂)₁₃—CO₂ | H, EDG, EWG |
| Cl, Br | (CH₂)₁₄—CO₂ | H, EDG, EWG |
| Cl, Br | (CH₂)₁₅—CO₂ | H, EDG, EWG |
| Cl, Br | (CH₂)₁₆—CO₂ | H, EDG, EWG |
| Cl, Br | (CH₂)₁₇—CO₂ | H, EDG, EWG |
| Cl, Br | (CH₂)₁₈—CO₂ | H, EDG, EWG |
| Cl, Br | CH₂—NH₂ | H, EDG, EWG |
| Cl, Br | (CH₂)₂—NH₂ | H, EDG, EWG |
| Cl, Br | (CH₂)₃—NH₂ | H, EDG, EWG |
| Cl, Br | (CH₂)₄—NH₂ | H, EDG, EWG |
| Cl, Br | (CH₂)₅—NH₂ | H, EDG, EWG |
| Cl, Br | (CH₂)₆—NH₂ | H, EDG, EWG |
| Cl, Br | (CH₂)₇—NH₂ | H, EDG, EWG |
| Cl, Br | (CH₂)₈—NH₂ | H, EDG, EWG |
| Cl, Br | (CH₂)₉—NH₂ | H, EDG, EWG |
| Cl, Br | (CH₂)₁₀—NH₂ | H, EDG, EWG |
| Cl, Br | (CH₂)₁₁—NH₂ | H, EDG, EWG |
| Cl, Br | (CH₂)₁₂—NH₂ | H, EDG, EWG |
| Cl, Br | (CH₂)₁₃—NH₂ | H, EDG, EWG |
| Cl, Br | (CH₂)₁₄—NH₂ | H, EDG, EWG |
| Cl, Br | (CH₂)₁₅—NH₂ | H, EDG, EWG |
| Cl, Br | (CH₂)₁₆—NH₂ | H, EDG, EWG |
| Cl, Br | (CH₂)₁₇—NH₂ | H, EDG, EWG |
| Cl, Br | (CH₂)₁₈—NH₂ | H, EDG, EWG |

*Each alkyl chain is optionally branched with an alkyl chain, aryl ring, heteroaryl, aralkyl group, or unsaturation at any position on the chain.
**The phenyl, benzyl, or naphthyl ring is optionally ortho-, meta-, or para-substituted with 1-3 substituents selected from halo, alkoxy, hydroxyl, CF3, NO2, NH2, NHR, or NR2.
***The R₂ group is H, any electron withdrawing group, or any electron donating group.

The A group is I, Cl, Br, OSO₂R, BF₄, ClO₄, or any pharmaceutically acceptable anion.

In various embodiments of compounds of Formula 3, X═Cl, R₁=Me; X═Cl, R₁=Et; X═Cl, R₁=n-Bu, R₂═H, SO₂NH₂, and A=I, ClO₄; X═Cl, R₁═(CH₂)₆CH₃, R₂═SO₂CH₃, and A=ClO₄; X═Cl, R1═(CH₂)₁₁CH₃, R₂═Cl, and A=BF₄; X═Cl, R₁=Ph, and A=BF₄ or —OSO₂R; X═Cl, R₁═CH₂CH═CH₂ and A=ClO₄; X═Cl, R₁═(CH₂)₃CH═CH₂, and A=ClO₄; X═Cl, R₁═CH₂OH, R₂=OEt, and A=ClO₄; X═Cl, R₁═(CH₂)₂OH and A=ClO₄; X═Cl, R₁═CH₂OMe, R₂═Cl, and A=ClO₄; X═Cl, R₁═CH₂O(CH₂)₃CH₃, R₂═Cl, and A=BF₄; X═Cl, R₁═CH₂OCH₂CH₃, R₂═Cl, and A=ClO₄; X═Cl, R₁═CH₂CH₂OMe and A=SbF₆; X═Cl, R₁=CH₂CH₂OEt and A=ClO₄; X═Cl, R₁═CH₂CH₂O(CH₂)₅CH₃ and A=ClO₄; X═Cl, R₁═(CH₂)₄OAc, R₂=CO₂Et, and A=ClO₄; X═Cl, R₁=CH₂CH₂O₂CNHPhh, R₂═CO₂Me or Cl, and A=ClO₄ or Br.

In various embodiments of compounds of Formula 3, where compounds are used in embodiments of the present invention, the compounds are not included where: X═Cl, R₁=Me; X═Cl, R₁=Et; X═Cl, R₁=n-Bu, R₂═H, SO₂NH₂, and A=I, ClO₄; X═Cl, R₁═(CH₂)₆CH₃, R₂═SO₂CH₃, and A=ClO₄; X═Cl, R₁═(CH₂)₁₁CH₃, R₂═Cl, and A=BF₄; X═Cl, R₁=Ph, and A=BF₄ or —OSO₂R; X═Cl, R₁═CH₂CH═CH₂ and A=ClO₄; X═Cl, R₁ ═(CH₂)₃CH═CH₂, and A=ClO₄; X═Cl, R₁═CH₂OH, R₂=OEt, and A=ClO₄; X═Cl, R₁ ═(CH₂)₂OH and A=ClO₄; X═Cl, R₁═CH₂OMe, R₂═Cl, and A=ClO₄; X═Cl, R₁═CH₂O(CH₂)₃CH₃, R₂═Cl, and A=BF₄; X═Cl, R₁═CH₂OCH₂CH₃, R₂═Cl, and A=ClO₄; X═Cl, R₁═CH₂CH₂OMe and A=SbF₆; X═Cl, R₁=CH₂CH₂OEt and A=ClO₄; X═Cl, R₁═CH₂CH₂O(CH₂)₅CH₃ and A=ClO₄; X═Cl, R₁═(CH₂)₄OAc, R₂=CO₂Et, and A=ClO₄; X═Cl, R₁=CH₂CH₂O₂CNHPhh, R₂═CO₂Me or Cl, and A=ClO₄ or Br.

Formula 4:

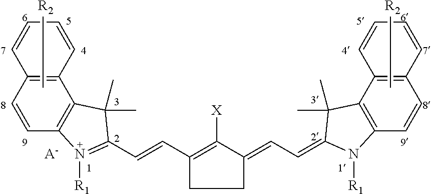

| X | R₁ | R₂*** |
|---|---|---|
| Cl, Br | Methyl | H, EDG, EWG |
| Cl, Br | Ethyl | H, EDG, EWG |
| Cl, Br | Propyl | H, EDG, EWG |
| Cl, Br | Butyl* | H, EDG, EWG |
| Cl, Br | Pentyl* | H, EDG, EWG |
| Cl, Br | Hexyl* | H, EDG, EWG |
| Cl, Br | Heptyl* | H, EDG, EWG |
| Cl, Br | Octyl* | H, EDG, EWG |
| Cl, Br | Nonyl* | H, EDG, EWG |
| Cl, Br | Decyl* | H, EDG, EWG |
| Cl, Br | Undecyl* | H, EDG, EWG |
| Cl, Br | Dodecyl* | H, EDG, EWG |
| Cl, Br | Tridecyl* | H, EDG, EWG |
| Cl, Br | Tetradecyl* | H, EDG, EWG |
| Cl, Br | Pentadecyl* | H, EDG, EWG |
| Cl, Br | Hexadecyl* | H, EDG, EWG |
| Cl, Br | Heptadecyl* | H, EDG, EWG |
| Cl, Br | Octadecyl* | H, EDG, EWG |
| Cl, Br | Phenyl** | H, EDG, EWG |
| Cl, Br | Benzyl** | H, EDG, EWG |
| Cl, Br | Naphthyl** | H, EDG, EWG |
| Cl, Br | CH₂—SO₃⁻ | H, EDG, EWG |
| Cl, Br | (CH₂)₂—SO₃⁻ | H, EDG, EWG |
| Cl, Br | (CH₂)₃—SO₃⁻ | H, EDG, EWG |
| Cl, Br | (CH₂)₄—SO₃⁻ | H, EDG, EWG |
| Cl, Br | (CH₂)₅—SO₃⁻ | H, EDG, EWG |
| Cl, Br | (CH₂)₆—SO₃⁻ | H, EDG, EWG |
| Cl, Br | (CH₂)₇—SO₃⁻ | H, EDG, EWG |
| Cl, Br | (CH₂)₈—SO₃⁻ | H, EDG, EWG |
| Cl, Br | (CH₂)₉—SO₃⁻ | H, EDG, EWG |
| Cl, Br | (CH₂)₁₀—SO₃⁻ | H, EDG, EWG |
| Cl, Br | (CH₂)₁₁—SO₃⁻ | H, EDG, EWG |
| Cl, Br | (CH₂)₁₂—SO₃⁻ | H, EDG, EWG |
| Cl, Br | (CH₂)₁₃—SO₃⁻ | H, EDG, EWG |
| Cl, Br | (CH₂)₁₄—SO₃⁻ | H, EDG, EWG |
| Cl, Br | (CH₂)₁₅—SO₃⁻ | H, EDG, EWG |
| Cl, Br | (CH₂)₁₆—SO₃⁻ | H, EDG, EWG |
| Cl, Br | (CH₂)₁₇—SO₃⁻ | H, EDG, EWG |
| Cl, Br | (CH₂)₁₈—SO₃⁻ | H, EDG, EWG |
| Cl, Br | CH₂—CO₂ | H, EDG, EWG |
| Cl, Br | (CH₂)₂—CO₂ | H, EDG, EWG |
| Cl, Br | (CH₂)₃—CO₂ | H, EDG, EWG |
| Cl, Br | (CH₂)₄—CO₂ | H, EDG, EWG |
| Cl, Br | (CH₂)₅—CO₂ | H, EDG, EWG |
| Cl, Br | (CH₂)₆—CO₂ | H, EDG, EWG |
| Cl, Br | (CH₂)₇—CO₂ | H, EDG, EWG |
| Cl, Br | (CH₂)₈—CO₂ | H, EDG, EWG |
| Cl, Br | (CH₂)₉—CO₂ | H, EDG, EWG |
| Cl, Br | (CH₂)₁₀—CO₂ | H, EDG, EWG |
| Cl, Br | (CH₂)₁₁—CO₂ | H, EDG, EWG |
| Cl, Br | (CH₂)₁₂—CO₂ | H, EDG, EWG |
| Cl, Br | (CH₂)₁₃—CO₂ | H, EDG, EWG |
| Cl, Br | (CH₂)₁₄—CO₂ | H, EDG, EWG |
| Cl, Br | (CH₂)₁₅—CO₂ | H, EDG, EWG |
| Cl, Br | (CH₂)₁₆—CO₂ | H, EDG, EWG |
| Cl, Br | (CH₂)₁₇—CO₂ | H, EDG, EWG |
| Cl, Br | (CH₂)₁₈—CO₂ | H, EDG, EWG |
| Cl, Br | CH₂—NH₂ | H, EDG, EWG |
| Cl, Br | (CH₂)₂—NH₂ | H, EDG, EWG |
| Cl, Br | (CH₂)₃—NH₂ | H, EDG, EWG |
| Cl, Br | (CH₂)₄—NH₂ | H, EDG, EWG |
| Cl, Br | (CH₂)₅—NH₂ | H, EDG, EWG |
| Cl, Br | (CH₂)₆—NH₂ | H, EDG, EWG |
| Cl, Br | (CH₂)₇—NH₂ | H, EDG, EWG |
| Cl, Br | (CH₂)₈—NH₂ | H, EDG, EWG |
| Cl, Br | (CH₂)₉—NH₂ | H, EDG, EWG |
| Cl, Br | (CH₂)₁₀—NH₂ | H, EDG, EWG |
| Cl, Br | (CH₂)₁₁—NH₂ | H, EDG, EWG |
| Cl, Br | (CH₂)₁₂—NH₂ | H, EDG, EWG |
| Cl, Br | (CH₂)₁₃—NH₂ | H, EDG, EWG |
| Cl, Br | (CH₂)₁₄—NH₂ | H, EDG, EWG |
| Cl, Br | (CH₂)₁₅—NH₂ | H, EDG, EWG |
| Cl, Br | (CH₂)₁₆—NH₂ | H, EDG, EWG |
| Cl, Br | (CH₂)₁₇—NH₂ | H, EDG, EWG |
| Cl, Br | (CH₂)₁₈—NH₂ | H, EDG, EWG |

*Each alkyl chain is optionally branched with an alkyl chain, aryl ring, heteroaryl, aralkyl group, or unsaturation at any position on the chain.
**The phenyl, benzyl, or naphthyl ring is optionally ortho-, meta-, or para-substituted with 1-3 substituents selected from halo, alkoxy, hydroxyl, CF3, NO2, NH2, NHR, or NR2.
***The R₂ group is H, any electron withdrawing group, or any electron donating group.

The A group is I, Cl, Br, OSO₂R, BF₄, ClO₄, or any pharmaceutically acceptable anion.

In various embodiments of compounds of Formula 4, X═Cl, R₁=Me, R₂═H or CH₂OAc, and A=SbF₆, —CO₂(CF₂)₂CF₃, —OSO₂(CF₂)₃CF₃, —OSO₂C₆H₄CH₃; X═Cl, R₁=Et, and A=ClO₄ or I; X═Cl, R₁=n-Pr, and A=PF₆⁻, OSO₂C₆H₄CH₃, or Cl; X═Cl, R₁=n-Bu, and A=PF₆⁻, OSO₂C₆H₄CH₃, Br, or ClO₄; X═Cl, R₁═—(CH₂)₉CH₃, and A=OSO₂CF₃; X═Cl, R₁═—CH₂OPh and A=ClO₄; X═Cl, R₁═—CH₂CH₂OMe, and A=N(SO₂CF₃)₂, X═Cl, R₁═—CH₂CH₂OH, and A=Br; X═Cl, R₁═—(CH₂)₅CO₂H and A=—OSO₂R; and X═Cl, R₁═—(CH₂)₄CH═CH₂ and A=ClO₄.

In embodiments of compounds of Formula 4, when compounds are used in embodiments of the present invention, compounds of Formula 4 do not include those compounds where: X═Cl, R₁=Me, R₂═H or CH₂OAc, and A=SbF₆, —CO₂(CF₂)₂CF₃, —OSO₂(CF₂)₃CF₃, —OSO₂C6H₄CH₃; X═Cl, R₁=Et, and A=ClO₄ or I; X═Cl, R₁=n-Pr, and A=PF₆⁻, OSO₂C6H₄CH₃, or Cl; X═Cl, R₁=n-Bu, and A=PF₆, OSO₂C6H₄CH₃, Br, or ClO₄; X═Cl, R₁═—(CH₂)₉CH₃, and A=OSO₂CF₃; X═Cl, R₁═—CH₂OPh and A=ClO₄—; X═Cl, R₁═—CH₂CH₂OMe, and A=N(SO₂CF₃)₂; X═Cl, R₁═—CH₂CH₂OH, and A=Br; X═Cl, R₁═—(CH₂)₅CO₂H and A=—OSO₂R; and X═Cl, R₁═—(CH₂)₄CH═CH₂ and A=ClO₄.

Formula 5:

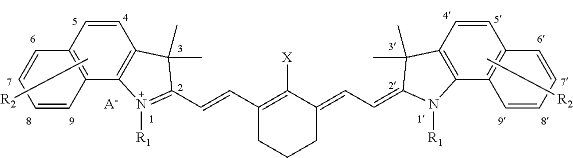

| X | R₁ | R₂*** |
|---|---|---|
| Cl, Br | Methyl | H, EDG, EWG |
| Cl, Br | Ethyl | H, EDG, EWG |
| Cl, Br | Propyl | H, EDG, EWG |
| Cl, Br | Butyl* | H, EDG, EWG |
| Cl, Br | Pentyl* | H, EDG, EWG |
| Cl, Br | Hexyl* | H, EDG, EWG |
| Cl, Br | Heptyl* | H, EDG, EWG |
| Cl, Br | Octyl* | H, EDG, EWG |
| Cl, Br | Nonyl* | H, EDG, EWG |
| Cl, Br | Decyl* | H, EDG, EWG |
| Cl, Br | Undecyl* | H, EDG, EWG |
| Cl, Br | Dodecyl* | H, EDG, EWG |
| Cl, Br | Tridecyl* | H, EDG, EWG |
| Cl, Br | Tetradecyl* | H, EDG, EWG |
| Cl, Br | Pentadecyl* | H, EDG, EWG |
| Cl, Br | Hexadecyl* | H, EDG, EWG |
| Cl, Br | Heptadecyl* | H, EDG, EWG |
| Cl, Br | Octadecyl* | H, EDG, EWG |
| Cl, Br | Phenyl** | H, EDG, EWG |
| Cl, Br | Benzyl** | H, EDG, EWG |
| Cl, Br | Naphthyl** | H, EDG, EWG |
| Cl, Br | CH₂—SO₃⁻ | H, EDG, EWG |
| Cl, Br | (CH₂)₂—SO₃⁻ | H, EDG, EWG |
| Cl, Br | (CH₂)₃—SO₃⁻ | H, EDG, EWG |
| Cl, Br | (CH₂)₄—SO₃⁻ | H, EDG, EWG |
| Cl, Br | (CH₂)₅—SO₃⁻ | H, EDG, EWG |
| Cl, Br | (CH₂)₆—SO₃⁻ | H, EDG, EWG |
| Cl, Br | (CH₂)₇—SO₃⁻ | H, EDG, EWG |
| Cl, Br | (CH₂)₈—SO₃⁻ | H, EDG, EWG |
| Cl, Br | (CH₂)₉—SO₃⁻ | H, EDG, EWG |
| Cl, Br | (CH₂)₁₀—SO₃⁻ | H, EDG, EWG |
| Cl, Br | (CH₂)₁₁—SO₃⁻ | H, EDG, EWG |
| Cl, Br | (CH₂)₁₂—SO₃⁻ | H, EDG, EWG |
| Cl, Br | (CH₂)₁₃—SO₃⁻ | H, EDG, EWG |
| Cl, Br | (CH₂)₁₄—SO₃⁻ | H, EDG, EWG |
| Cl, Br | (CH₂)₁₅—SO₃⁻ | H, EDG, EWG |
| Cl, Br | (CH₂)₁₆—SO₃⁻ | H, EDG, EWG |
| Cl, Br | (CH₂)₁₇—SO₃⁻ | H, EDG, EWG |
| Cl, Br | (CH₂)₁₈—SO₃⁻ | H, EDG, EWG |
| Cl, Br | CH₂—CO₂ | H, EDG, EWG |
| Cl, Br | (CH₂)₂—CO₂ | H, EDG, EWG |
| Cl, Br | (CH₂)₃—CO₂ | H, EDG, EWG |
| Cl, Br | (CH₂)₄—CO₂ | H, EDG, EWG |
| Cl, Br | (CH₂)₅—CO₂ | H, EDG, EWG |
| Cl, Br | (CH₂)₆—CO₂ | H, EDG, EWG |
| Cl, Br | (CH₂)₇—CO₂ | H, EDG, EWG |
| Cl, Br | (CH₂)₈—CO₂ | H, EDG, EWG |
| Cl, Br | (CH₂)₉—CO₂ | H, EDG, EWG |
| Cl, Br | (CH₂)₁₀—CO₂ | H, EDG, EWG |
| Cl, Br | (CH₂)₁₁—CO₂ | H, EDG, EWG |
| Cl, Br | (CH₂)₁₂—CO₂ | H, EDG, EWG |
| Cl, Br | (CH₂)₁₃—CO₂ | H, EDG, EWG |
| Cl, Br | (CH₂)₁₄—CO₂ | H, EDG, EWG |
| Cl, Br | (CH₂)₁₅—CO₂ | H, EDG, EWG |
| Cl, Br | (CH₂)₁₆—CO₂ | H, EDG, EWG |
| Cl, Br | (CH₂)₁₇—CO₂ | H, EDG, EWG |
| Cl, Br | (CH₂)₁₈—CO₂ | H, EDG, EWG |
| Cl, Br | CH₂—NH₂ | H, EDG, EWG |
| Cl, Br | (CH₂)₂—NH₂ | H, EDG, EWG |
| Cl, Br | (CH₂)₃—NH₂ | H, EDG, EWG |
| Cl, Br | (CH₂)₄—NH₂ | H, EDG, EWG |
| Cl, Br | (CH₂)₅—NH₂ | H, EDG, EWG |
| Cl, Br | (CH₂)₆—NH₂ | H, EDG, EWG |
| Cl, Br | (CH₂)₇—NH₂ | H, EDG, EWG |
| Cl, Br | (CH₂)₈—NH₂ | H, EDG, EWG |
| Cl, Br | (CH₂)₉—NH₂ | H, EDG, EWG |
| Cl, Br | (CH₂)₁₀—NH₂ | H, EDG, EWG |
| Cl, Br | (CH₂)₁₁—NH₂ | H, EDG, EWG |
| Cl, Br | (CH₂)₁₂—NH₂ | H, EDG, EWG |
| Cl, Br | (CH₂)₁₃—NH₂ | H, EDG, EWG |
| Cl, Br | (CH₂)₁₄—NH₂ | H, EDG, EWG |
| Cl, Br | (CH₂)₁₅—NH₂ | H, EDG, EWG |
| Cl, Br | (CH₂)₁₆—NH₂ | H, EDG, EWG |
| Cl, Br | (CH₂)₁₇—NH₂ | H, EDG, EWG |
| Cl, Br | (CH₂)₁₈—NH₂ | H, EDG, EWG |

*Each alkyl chain is optionally branched with an alkyl chain, aryl ring, heteroaryl, aralkyl group, or unsaturation at any position on the chain.
**The phenyl, benzyl, or naphthyl ring is optionally ortho-, meta-, or para-substituted with 1-3 substituents selected from halo, alkoxy, hydroxyl, CF3, NO2, NH2, NHR, or NR2.
***The R₂ group is H, any electron withdrawing group, or any electron donating group.

The A group is I, Cl, Br, OSO₂R, BF₄, ClO₄, or any pharmaceutically acceptable anion.

In one embodiment of compounds of Formula 5, X═Cl, R₁=Me, R₂═H, and A=SbF₆ and —OSO₂C6H₄CH₃. In one embodiments of compounds of Formula 5, when compounds are used in methods of the present invention, compounds of Formula 5 do not include those where: X═Cl, R₁=Me, R₂═H, and A=SbF₆ and —OSO₂C₆H₄CH₃.

Formula 6:

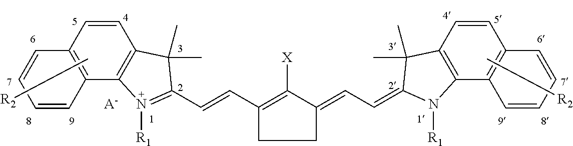

| X | R₁ | R₂*** |
|---|---|---|
| Cl, Br | Methyl | H, EDG, EWG |
| Cl, Br | Ethyl | H, EDG, EWG |
| Cl, Br | Propyl | H, EDG, EWG |
| Cl, Br | Butyl* | H, EDG, EWG |
| Cl, Br | Pentyl* | H, EDG, EWG |
| Cl, Br | Hexyl* | H, EDG, EWG |
| Cl, Br | Heptyl* | H, EDG, EWG |
| Cl, Br | Octyl* | H, EDG, EWG |
| Cl, Br | Nonyl* | H, EDG, EWG |
| Cl, Br | Decyl* | H, EDG, EWG |
| Cl, Br | Undecyl* | H, EDG, EWG |
| Cl, Br | Dodecyl* | H, EDG, EWG |
| Cl, Br | Tridecyl* | H, EDG, EWG |
| Cl, Br | Tetradecyl* | H, EDG, EWG |
| Cl, Br | Pentadecyl* | H, EDG, EWG |
| Cl, Br | Hexadecyl* | H, EDG, EWG |
| Cl, Br | Heptadecyl* | H, EDG, EWG |
| Cl, Br | Octadecyl* | H, EDG, EWG |
| Cl, Br | Phenyl** | H, EDG, EWG |
| Cl, Br | Benzyl** | H, EDG, EWG |
| Cl, Br | Naphthyl** | H, EDG, EWG |
| Cl, Br | CH₂—SO₃⁻ | H, EDG, EWG |
| Cl, Br | (CH₂)₂—SO₃⁻ | H, EDG, EWG |
| Cl, Br | (CH₂)₃—SO₃⁻ | H, EDG, EWG |
| Cl, Br | (CH₂)₄—SO₃⁻ | H, EDG, EWG |
| Cl, Br | (CH₂)₅—SO₃⁻ | H, EDG, EWG |
| Cl, Br | (CH₂)₆—SO₃⁻ | H, EDG, EWG |
| Cl, Br | (CH₂)₇—SO₃⁻ | H, EDG, EWG |
| Cl, Br | (CH₂)₈—SO₃⁻ | H, EDG, EWG |
| Cl, Br | (CH₂)₉—SO₃⁻ | H, EDG, EWG |
| Cl, Br | (CH₂)₁₀—SO₃⁻ | H, EDG, EWG |
| Cl, Br | (CH₂)₁₁—SO₃⁻ | H, EDG, EWG |
| Cl, Br | (CH₂)₁₂—SO₃⁻ | H, EDG, EWG |
| Cl, Br | (CH₂)₁₃—SO₃⁻ | H, EDG, EWG |
| Cl, Br | (CH₂)₁₄—SO₃⁻ | H, EDG, EWG |
| Cl, Br | (CH₂)₁₅—SO₃⁻ | H, EDG, EWG |
| Cl, Br | (CH₂)₁₆—SO₃⁻ | H, EDG, EWG |
| Cl, Br | (CH₂)₁₇—SO₃⁻ | H, EDG, EWG |
| Cl, Br | (CH₂)₁₈—SO₃⁻ | H, EDG, EWG |
| Cl, Br | CH₂—CO₂ | H, EDG, EWG |
| Cl, Br | (CH₂)₂—CO₂ | H, EDG, EWG |
| Cl, Br | (CH₂)₃—CO₂ | H, EDG, EWG |
| Cl, Br | (CH₂)₄—CO₂ | H, EDG, EWG |
| Cl, Br | (CH₂)₅—CO₂ | H, EDG, EWG |
| Cl, Br | (CH₂)₆—CO₂ | H, EDG, EWG |
| Cl, Br | (CH₂)₇—CO₂ | H, EDG, EWG |
| Cl, Br | (CH₂)₈—CO₂ | H, EDG, EWG |
| Cl, Br | (CH₂)₉—CO₂ | H, EDG, EWG |
| Cl, Br | (CH₂)₁₀—CO₂ | H, EDG, EWG |
| Cl, Br | (CH₂)₁₁—CO₂ | H, EDG, EWG |
| Cl, Br | (CH₂)₁₂—CO₂ | H, EDG, EWG |
| Cl, Br | (CH₂)₁₃—CO₂ | H, EDG, EWG |
| Cl, Br | (CH₂)₁₄—CO₂ | H, EDG, EWG |
| Cl, Br | (CH₂)₁₅—CO₂ | H, EDG, EWG |
| Cl, Br | (CH₂)₁₆—CO₂ | H, EDG, EWG |
| Cl, Br | (CH₂)₁₇—CO₂ | H, EDG, EWG |
| Cl, Br | (CH₂)₁₈—CO₂ | H, EDG, EWG |
| Cl, Br | CH₂—NH₂ | H, EDG, EWG |
| Cl, Br | (CH₂)₂—NH₂ | H, EDG, EWG |
| Cl, Br | (CH₂)₃—NH₂ | H, EDG, EWG |
| Cl, Br | (CH₂)₄—NH₂ | H, EDG, EWG |
| Cl, Br | (CH₂)₅—NH₂ | H, EDG, EWG |
| Cl, Br | (CH₂)₆—NH₂ | H, EDG, EWG |
| Cl, Br | (CH₂)₇—NH₂ | H, EDG, EWG |
| Cl, Br | (CH₂)₈—NH₂ | H, EDG, EWG |
| Cl, Br | (CH₂)₉—NH₂ | H, EDG, EWG |
| Cl, Br | (CH₂)₁₀—NH₂ | H, EDG, EWG |
| Cl, Br | (CH₂)₁₁—NH₂ | H, EDG, EWG |
| Cl, Br | (CH₂)₁₂—NH₂ | H, EDG, EWG |
| Cl, Br | (CH₂)₁₃—NH₂ | H, EDG, EWG |
| Cl, Br | (CH₂)₁₄—NH₂ | H, EDG, EWG |
| Cl, Br | (CH₂)₁₅—NH₂ | H, EDG, EWG |
| Cl, Br | (CH₂)₁₆—NH₂ | H, EDG, EWG |
| Cl, Br | (CH₂)₁₇—NH₂ | H, EDG, EWG |
| Cl, Br | (CH₂)₁₈—NH₂ | H, EDG, EWG |

*Each alkyl chain is optionally branched with an alkyl chain, aryl ring, heteroaryl, aralkyl group, or unsaturation at any position on the chain.
**The phenyl, benzyl, or naphthyl ring is optionally ortho-, meta-, or para-substituted with 1-3 substituents selected from halo, alkoxy, hydroxyl, CF3, NO2, NH2, NHR, or NR2.
***The R₂ group is H, any electron withdrawing group, or any electron donating group.

The A group is I, Cl, Br, OSO₂R, BF₄, ClO₄, or any pharmaceutically acceptable anion.

In embodiments of compounds of Formula 6, X═Cl, R₁=n-Bu, R₂═H, and A=SbF₆; X═Cl, R₁═—CH₂OMe, and A=Cl; X═Cl, R₁═—(CH₂)₂COOEt, and A=ClO₄. In embodiments of compounds of Formula 6, when compounds of Formula 6 are used in methods of the present invention, the compounds are not included where: X═Cl, R₁=n-Bu, R₂═H, and A=SbF₆; X═Cl, R₁═—CH₂OMe, and A=Cl; X═Cl, R₁═—(CH₂)₂COOEt, and A=ClO₄.

Formula 7:

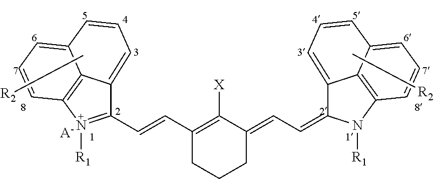

| X | R₁ | R₂*** |
|---|---|---|
| Cl, Br | Methyl | H, EDG, EWG |
| Cl, Br | Ethyl | H, EDG, EWG |
| Cl, Br | Propyl | H, EDG, EWG |
| Cl, Br | Butyl* | H, EDG, EWG |
| Cl, Br | Pentyl* | H, EDG, EWG |
| Cl, Br | Hexyl* | H, EDG, EWG |
| Cl, Br | Heptyl* | H, EDG, EWG |
| Cl, Br | Octyl* | H, EDG, EWG |
| Cl, Br | Nonyl* | H, EDG, EWG |
| Cl, Br | Decyl* | H, EDG, EWG |
| Cl, Br | Undecyl* | H, EDG, EWG |
| Cl, Br | Dodecyl* | H, EDG, EWG |
| Cl, Br | Tridecyl* | H, EDG, EWG |
| Cl, Br | Tetradecyl* | H, EDG, EWG |
| Cl, Br | Pentadecyl* | H, EDG, EWG |
| Cl, Br | Hexadecyl* | H, EDG, EWG |
| Cl, Br | Heptadecyl* | H, EDG, EWG |
| Cl, Br | Octadecyl* | H, EDG, EWG |
| Cl, Br | Phenyl** | H, EDG, EWG |
| Cl, Br | Benzyl** | H, EDG, EWG |
| Cl, Br | Naphthyl** | H, EDG, EWG |
| Cl, Br | CH₂—SO₃⁻ | H, EDG, EWG |
| Cl, Br | (CH₂)₂—SO₃⁻ | H, EDG, EWG |
| Cl, Br | (CH₂)₃—SO₃⁻ | H, EDG, EWG |
| Cl, Br | (CH₂)₄—SO₃⁻ | H, EDG, EWG |
| Cl, Br | (CH₂)₅—SO₃⁻ | H, EDG, EWG |
| Cl, Br | (CH₂)₆—SO₃⁻ | H, EDG, EWG |
| Cl, Br | (CH₂)₇—SO₃⁻ | H, EDG, EWG |
| Cl, Br | (CH₂)₈—SO₃⁻ | H, EDG, EWG |
| Cl, Br | (CH₂)₉—SO₃⁻ | H, EDG, EWG |
| Cl, Br | (CH₂)₁₀—SO₃⁻ | H, EDG, EWG |
| Cl, Br | (CH₂)₁₁—SO₃⁻ | H, EDG, EWG |
| Cl, Br | (CH₂)₁₂—SO₃⁻ | H, EDG, EWG |
| Cl, Br | (CH₂)₁₃—SO₃⁻ | H, EDG, EWG |
| Cl, Br | (CH₂)₁₄—SO₃⁻ | H, EDG, EWG |
| Cl, Br | (CH₂)₁₅—SO₃⁻ | H, EDG, EWG |
| Cl, Br | (CH₂)₁₆—SO₃⁻ | H, EDG, EWG |
| Cl, Br | (CH₂)₁₇—SO₃⁻ | H, EDG, EWG |
| Cl, Br | (CH₂)₁₈—SO₃⁻ | H, EDG, EWG |
| Cl, Br | CH₂—CO₂ | H, EDG, EWG |
| Cl, Br | (CH₂)₂—CO₂ | H, EDG, EWG |
| Cl, Br | (CH₂)₃—CO₂ | H, EDG, EWG |
| Cl, Br | (CH₂)₄—CO₂ | H, EDG, EWG |
| Cl, Br | (CH₂)₅—CO₂ | H, EDG, EWG |
| Cl, Br | (CH₂)₆—CO₂ | H, EDG, EWG |
| Cl, Br | (CH₂)₇—CO₂ | H, EDG, EWG |
| Cl, Br | (CH₂)₈—CO₂ | H, EDG, EWG |
| Cl, Br | (CH₂)₉—CO₂ | H, EDG, EWG |
| Cl, Br | (CH₂)₁₀—CO₂ | H, EDG, EWG |
| Cl, Br | (CH₂)₁₁—CO₂ | H, EDG, EWG |
| Cl, Br | (CH₂)₁₂—CO₂ | H, EDG, EWG |
| Cl, Br | (CH₂)₁₃—CO₂ | H, EDG, EWG |
| Cl, Br | (CH₂)₁₄—CO₂ | H, EDG, EWG |
| Cl, Br | (CH₂)₁₅—CO₂ | H, EDG, EWG |
| Cl, Br | (CH₂)₁₆—CO₂ | H, EDG, EWG |
| Cl, Br | (CH₂)₁₇—CO₂ | H, EDG, EWG |
| Cl, Br | (CH₂)₁₈—CO₂ | H, EDG, EWG |
| Cl, Br | CH₂—NH₂ | H, EDG, EWG |
| Cl, Br | (CH₂)₂—NH₂ | H, EDG, EWG |
| Cl, Br | (CH₂)₃—NH₂ | H, EDG, EWG |
| Cl, Br | (CH₂)₄—NH₂ | H, EDG, EWG |
| Cl, Br | (CH₂)₅—NH₂ | H, EDG, EWG |
| Cl, Br | (CH₂)₆—NH₂ | H, EDG, EWG |
| Cl, Br | (CH₂)₇—NH₂ | H, EDG, EWG |
| Cl, Br | (CH₂)₈—NH₂ | H, EDG, EWG |
| Cl, Br | (CH₂)₉—NH₂ | H, EDG, EWG |
| Cl, Br | (CH₂)₁₀—NH₂ | H, EDG, EWG |
| Cl, Br | (CH₂)₁₁—NH₂ | H, EDG, EWG |
| Cl, Br | (CH₂)₁₂—NH₂ | H, EDG, EWG |
| Cl, Br | (CH₂)₁₃—NH₂ | H, EDG, EWG |
| Cl, Br | (CH₂)₁₄—NH₂ | H, EDG, EWG |
| Cl, Br | (CH₂)₁₅—NH₂ | H, EDG, EWG |
| Cl, Br | (CH₂)₁₆—NH₂ | H, EDG, EWG |
| Cl, Br | (CH₂)₁₇—NH₂ | H, EDG, EWG |
| Cl, Br | (CH₂)₁₈—NH₂ | H, EDG, EWG |

*Each alkyl chain is optionally branched with an alkyl chain, aryl ring, heteroaryl, aralkyl group, or unsaturation at any position on the chain.
**The phenyl, benzyl, or naphthyl ring is optionally ortho-, meta-, or para-substituted with 1-3 substituents selected from halo, alkoxy, hydroxyl, CF3, NO2, NH2, NHR, or NR2.
***The R₂ group is H, any electron withdrawing group, or any electron donating group.

The A group is I, Cl, Br, OSO₂R, BF₄, ClO₄, or any pharmaceutically acceptable anion.

In embodiments of compounds of Formula 7, X═Cl, R₁=n-Bu, R₂═H or Cl, and A=BF₄ or PF₆; X═Cl, R₁=Et, R₂═H, and A=I or Cl; X═Cl, R₁=decyl, R₂═H or Me, and A=Cl or BF₄; X═—C, R₁=dodecyl, R₂═H, Cl, SPh, or OMe, and A=Cl, BF₄; X═Cl, R₁=allyl, and A=I; X═Cl, R₁=octadecyl, and A=ClO₄; and X═Cl, R₁═—(CH₂)₂COOH, and A=BF₄.

In embodiments of compounds of Formula 7, when compounds of Formula 7 are used in methods of the present invention, the compounds are not included where: X═Cl, R₁=n-Bu, R₂═H or Cl, and A=BF₄ or PF₆; X═Cl, R₁=Et, R₂═H, and A=I or Cl; X═Cl, R₁=decyl, R₂═H or Me, and A=Cl or BF₄; X═—C, R₁=dodecyl, R₂═H, Cl, SPh, or OMe, and A=Cl, BF₄; X═Cl, R₁=allyl, and A=I; X═Cl, R=octadecyl, and A=ClO₄; and X═Cl, R₁═—(CH₂)₂COOH, and A=BF₄.

Formula 8:

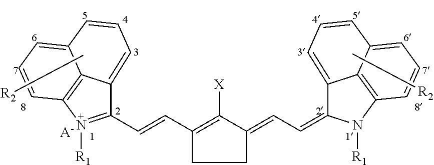

| X | R₁ | R₂*** |
|---|---|---|
| Cl, Br | Methyl | H, EDG, EWG |
| Cl, Br | Ethyl | H, EDG, EWG |
| Cl, Br | Propyl | H, EDG, EWG |
| Cl, Br | Butyl* | H, EDG, EWG |
| Cl, Br | Pentyl* | H, EDG, EWG |
| Cl, Br | Hexyl* | H, EDG, EWG |
| Cl, Br | Heptyl* | H, EDG, EWG |
| Cl, Br | Octyl* | H, EDG, EWG |
| Cl, Br | Nonyl* | H, EDG, EWG |
| Cl, Br | Decyl* | H, EDG, EWG |
| Cl, Br | Undecyl* | H, EDG, EWG |
| Cl, Br | Dodecyl* | H, EDG, EWG |
| Cl, Br | Tridecyl* | H, EDG, EWG |
| Cl, Br | Tetradecyl* | H, EDG, EWG |
| Cl, Br | Pentadecyl* | H, EDG, EWG |
| Cl, Br | Hexadecyl* | H, EDG, EWG |
| Cl, Br | Heptadecyl* | H, EDG, EWG |
| Cl, Br | Octadecyl* | H, EDG, EWG |
| Cl, Br | Phenyl** | H, EDG, EWG |
| Cl, Br | Benzyl** | H, EDG, EWG |
| Cl, Br | Naphthyl** | H, EDG, EWG |
| Cl, Br | CH₂—SO₃⁻ | H, EDG, EWG |
| Cl, Br | (CH₂)₂—SO₃⁻ | H, EDG, EWG |
| Cl, Br | (CH₂)₃—SO₃⁻ | H, EDG, EWG |
| Cl, Br | (CH₂)₄—SO₃⁻ | H, EDG, EWG |
| Cl, Br | (CH₂)₅—SO₃⁻ | H, EDG, EWG |
| Cl, Br | (CH₂)₆—SO₃⁻ | H, EDG, EWG |
| Cl, Br | (CH₂)₇—SO₃⁻ | H, EDG, EWG |
| Cl, Br | (CH₂)₈—SO₃⁻ | H, EDG, EWG |
| Cl, Br | (CH₂)₉—SO₃⁻ | H, EDG, EWG |
| Cl, Br | (CH₂)₁₀—SO₃⁻ | H, EDG, EWG |
| Cl, Br | (CH₂)₁₁—SO₃⁻ | H, EDG, EWG |
| Cl, Br | (CH₂)₁₂—SO₃⁻ | H, EDG, EWG |
| Cl, Br | (CH₂)₁₃—SO₃⁻ | H, EDG, EWG |
| Cl, Br | (CH₂)₁₄—SO₃⁻ | H, EDG, EWG |
| Cl, Br | (CH₂)₁₅—SO₃⁻ | H, EDG, EWG |
| Cl, Br | (CH₂)₁₆—SO₃⁻ | H, EDG, EWG |
| Cl, Br | (CH₂)₁₇—SO₃⁻ | H, EDG, EWG |
| Cl, Br | (CH₂)₁₈—SO₃⁻ | H, EDG, EWG |
| Cl, Br | CH₂—CO₂ | H, EDG, EWG |
| Cl, Br | (CH₂)₂—CO₂ | H, EDG, EWG |
| Cl, Br | (CH₂)₃—CO₂ | H, EDG, EWG |
| Cl, Br | (CH₂)₄—CO₂ | H, EDG, EWG |
| Cl, Br | (CH₂)₅—CO₂ | H, EDG, EWG |
| Cl, Br | (CH₂)₆—CO₂ | H, EDG, EWG |
| Cl, Br | (CH₂)₇—CO₂ | H, EDG, EWG |
| Cl, Br | (CH₂)₈—CO₂ | H, EDG, EWG |
| Cl, Br | (CH₂)₉—CO₂ | H, EDG, EWG |
| Cl, Br | (CH₂)₁₀—CO₂ | H, EDG, EWG |
| Cl, Br | (CH₂)₁₁—CO₂ | H, EDG, EWG |
| Cl, Br | (CH₂)₁₂—CO₂ | H, EDG, EWG |
| Cl, Br | (CH₂)₁₃—CO₂ | H, EDG, EWG |
| Cl, Br | (CH₂)₁₄—CO₂ | H, EDG, EWG |
| Cl, Br | (CH₂)₁₅—CO₂ | H, EDG, EWG |
| Cl, Br | (CH₂)₁₆—CO₂ | H, EDG, EWG |
| Cl, Br | (CH₂)₁₇—CO₂ | H, EDG, EWG |
| Cl, Br | (CH₂)₁₈—CO₂ | H, EDG, EWG |
| Cl, Br | CH₂—NH₂ | H, EDG, EWG |
| Cl, Br | (CH₂)₂—NH₂ | H, EDG, EWG |
| Cl, Br | (CH₂)₃—NH₂ | H, EDG, EWG |
| Cl, Br | (CH₂)₄—NH₂ | H, EDG, EWG |
| Cl, Br | (CH₂)₅—NH₂ | H, EDG, EWG |
| Cl, Br | (CH₂)₆—NH₂ | H, EDG, EWG |
| Cl, Br | (CH₂)₇—NH₂ | H, EDG, EWG |
| Cl, Br | (CH₂)₈—NH₂ | H, EDG, EWG |
| Cl, Br | (CH₂)₉—NH₂ | H, EDG, EWG |
| Cl, Br | (CH₂)₁₀—NH₂ | H, EDG, EWG |
| Cl, Br | (CH₂)₁₁—NH₂ | H, EDG, EWG |
| Cl, Br | (CH₂)₁₂—NH₂ | H, EDG, EWG |
| Cl, Br | (CH₂)₁₃—NH₂ | H, EDG, EWG |
| Cl, Br | (CH₂)₁₄—NH₂ | H, EDG, EWG |
| Cl, Br | (CH₂)₁₅—NH₂ | H, EDG, EWG |
| Cl, Br | (CH₂)₁₆—NH₂ | H, EDG, EWG |
| Cl, Br | (CH₂)₁₇—NH₂ | H, EDG, EWG |
| Cl, Br | (CH₂)₁₈—NH₂ | H, EDG, EWG |

*Each alkyl chain is optionally branched with an alkyl chain, aryl ring, heteroaryl, aralkyl group, or unsaturation at any position on the chain.
**The phenyl, benzyl, or naphthyl ring is optionally ortho-, meta-, or para-substituted with 1-3 substituents selected from halo, alkoxy, hydroxyl, CF3, NO2, NH2, NHR, or NR2.
***The R₂ group is H, any electron withdrawing group, or any electron donating group.

The A group is I, Cl, Br, OSO₂R, BF₄, ClO₄, or any pharmaceutically acceptable anion.

In various embodiments of compounds of Formula 8, X═Cl, R₁=Et, R₂═H or SPh, and A=Cl.

In various embodiments, when compounds of Formula 8 are used in methods of the present invention, the compounds are not included where: X═Cl, R₁=Et, R₂═H or SPh, and A=Cl.

In particular embodiments, the NIR organic carbocyanine dye used in the methods of the present invention is IR-780 or a derivative thereof. In particular embodiments, the NIR organic carbocyanine dye used in these methods is IR-783 or a derivative thereof.

In various embodiments, the NIR organic carbocyanine dye used in these methods is a near-infrared (NIR) heptamethine cyanine dye. In particular embodiments, the NIR heptamethine cyanine dye used in these methods is IR-783 or a derivative thereof. In particular embodiments, the NIR heptamethine cyanine dye used in these methods is MHI-148 or a derivative thereof.

Various embodiments of the present invention provide for a method of identifying a cancer cell or a tumor in a subject in need thereof. The method can comprise: providing a near-infrared (NIR) organic carbocyanine dye; administering the NIR organic carbocyanine dye to the subject; and imaging the subject to identify the cancer cell or tumor in the subject.

Various embodiments of the present invention provide for a method of detecting a cancer cell or a tumor in a subject in need thereof. The method can comprise: providing a near-infrared (NIR) organic carbocyanine dye; administering the NIR organic carbocyanine dye to the subject; and imaging the subject to detect the cancer cell or tumor in the subject.

Various embodiments of the present invention provide for a method of imaging a cancer cell or a tumor in a subject in need thereof. The method can comprise: providing a near-infrared (NIR) organic carbocyanine dye, administering the NIR organic carbocyanine dye to the subject; and imaging the subject to obtain an image of the cancer cell or tumor in the subject.

Various embodiments of the present invention provide for a method of locating a cancer cell or a tumor in a subject in need thereof. The method can comprise: providing a near-infrared (NIR) organic carbocyanine dye, administering the NIR organic carbocyanine dye to the subject; and imaging the subject to locate the cancer cell or tumor in the subject.

Various embodiments of the present invention provides for a method of isolating a cancer cell or a tumor in a subject in need thereof. The method can comprise: providing a biological sample from the subject; contacting the biological sample to a near-infrared (NIR) organic carbocyanine dye; separating a cancer cell or tumor based on the uptake of NIR organic carbocyanine dye in a cell or tumor, wherein a cell or tumor that uptakes the NIR organic carbocyanine dye is determined to be a cancer cell or tumor.

In various embodiments, the method of identifying, detecting, imaging, locating and/or isolating the cancer cell can comprise: providing a biological sample from the subject; contacting the biological sample with an NIR organic carbocyanine dye to form a mixture; and analyzing the mixture. In various embodiments, analyzing the mixture is performed by fluorescence microscopy. In various embodiments, analyzing the mixture is performed by using microfluidics apparatus. In various embodiments, analyzing the mixture is performed by flow cytometric analysis. In various embodiments, analyzing the mixture is performed by FACS.

In various embodiments, the mixture is processed through a chaotic mixing channel in a microfluidics apparatus or a flow cytometer. In particular embodiments, an overlaid polydimethylsiloxane chip with a serpentine chaotic mixing channel is used to encourage cell/dye contact frequency. Details of the chaotic mixing channel and the overlaid polydimethylsiloxane chip with a serpentine chaotic mixing channel are described by Wang et al., Highly Efficient Capture of Circulating Tumor Cells Using Nanostructured Silicon Substrates with Integrated Chaotic Micromixers. ANGEWANDTE CHEMIE INTERNATIONAL ED., which is incorporated by reference as though full set forth in its entirety.

In various embodiments, the mixture can be analyzed using apparatuses and methods described in U.S. Patent Publication Nos. 20100291584, 20090302228, and 20080281090, which are incorporated by reference as though fully set forth in its entirety.

In various embodiments, cells are separated from the mixture, and a light (e.g., laser) is directed at each cell, wherein a presence of an increased NIR fluorescent signal, relative to the background staining intensity, indicates the cell is a cancer cell and/or tumor cell, and the lack of an increased NIR fluorescent signal, relative to the background staining intensity indicates that the cell is not a tumor cell.

In various embodiments, single cells from the mixture flow through a detection element of the microfluidics apparatus whereby the light is directed to each cell, and a presence of an increased NIR fluorescent signal from the cell, relative to a background staining intensity, indicates the cell is a cancer or tumor cell, and the lack of an increased NIR fluorescent signal from the cell, relative to the background staining intensity indicates that the cell is not a tumor cell. In various embodiments, the microfluidics apparatus is a flow cytometer. In various embodiments, single cells from the mixture flow through an FACS system.

In various embodiments, the light used in these methods is adapted to excite an NIR organic carbocyanine dye taken up by a cell. In various embodiments, the light used in these methods is a laser adapted to excite an NIR organic carbocyanine dye taken up by a cell.

In various embodiments, each cell that is identified as a cancer cell or a tumor cell is separated from the mixture and can be further analyzed.

Various embodiments of the present invention provide for a method to conduct in situ pharmacokinetic and pharmacodynamic analyses of a molecule in a cancer cell or a tumor using an NIR organic carbocyanine dye-molecule conjugate or complex. The method comprises providing the NIR organic carbocyanine dye-molecule conjugate or complex; administering the NIR organic carbocyanine dye-molecule or complex to a subject; and determining the pharmacokinetics or pharmacodynamics of the molecule in the cancer cell, or the tumor tissue in the subject. With the dye serving as a tracer, distribution and clearance of the molecule being studied are reflected by the distribution, metabolism and clearance of the dye. The dye is selected based on its NIR fluorescence, which indicates the presence of the molecule being studied. The concentrations of the dye can be estimated by the light emission at certain NIR wavelengths based on the standard curves. The extinction coefficient of a dye is related directly to the concentration of a dye, thus by measuring the light absorption of a dye at the maximal emission wavelength one can determine how much of the drug is in tissue or cell. Typically, standard curves can be constructed first; thereafter, the reading from the sample with unknown concentration is used to extrapolate from the standard curve to determine the amount of dye is in the cell or tissue (see e.g., Yang et al. Clin Cancer Res. 2010) Various embodiments of the present invention provide for a method to tag a molecule with an NIR organic carbocyanine dye and to follow its movements in living subjects. The method comprises providing a NIR organic carbocyanine dye conjugated or complexed to the molecule; administering the NIR organic carbocyanine dye conjugate or complex to the living subject; and imaging the subject to follow the movement of the molecule in the living subject. In another embodiment, the method may comprise continuous imaging of the subject. With the dye serving as a tracer, movements of the molecule being studied are reflected by the movement of the dye which can be imaged by a NIR camera or detecting device. The dye is detected based on its NIR fluorescence, which correlate with the movement of the molecule being studied.

Various embodiments of the present invention provide for a method to increase the delivery of a molecule to a cancer cell. The method comprises providing an NIR organic carbocyanine dye conjugated to or complexed with a molecule; and administering the NIR organic carbocyanine dye conjugate or complex to the subject to increase the delivery of the molecule to the cancer cell in the subject. The dye serves as a carrier. As it is preferentially taken up and retained in cancer but not normal cells; the dye, conjugated or non-conjugated will be taken up by cancer and not normal cells, which can be tracked and the concentration determined by the extinction coefficient of the dye.

Various embodiments of the present invention provide for a method to tag a cell or a microorganism to follow their movements in a living subject. The method comprises providing a near-infrared (NIR) organic carbocyanine dye; allowing the uptake of the NIR organic carbocyanine dye by the cell or the microorganism; administering the cell containing the NIR organic carbocyanine dye or the microorganism containing the NIR organic carbocyanine dye to a living subject; imaging the subject to follow the movement of the cell or the microorganism in the living subject. In another embodiment, the method may comprise continuous imaging of the subject. The dye is trapped in the cell or organism and thus serves as a tracer of the cell or the organism. The dye is detected based on it NIR fluorescence, which indicates the movement and compartmentalization of the cell or the organism, which is loaded with the dye inside the subject.

In various embodiments, the cell is a cancer cell and the method comprises studying cancer metastases in the living subject. In various embodiments, the living subject is a conventional animal used in an animal model or a transgenic animal used in an animal model. In other embodiments, the living subject is a human subject. As the dye is preferentially taken up and retained by cancer cells, the dye is highly concentrated in the tumor cells after administration to the subject and the dye serves as a tracer. The dye is detected based on its NIR fluorescence, which indicates the location and the quantity of the dye which correlates with the size and location of the tumor or the location of the cancer cells.

Various embodiments of the present invention provide for a method to detect the changes in tumors from being well vascularized to poorly vascularized and necrotic after drug treatment. Since the NIR organic carbocyanine dye is distributed through systemic circulation, the NIR organic carbocyanine dye enters a well vascularized tumor easily and rapidly; and enters a poorly vascularized tumor slowly and will not reach the center of a necrotic tumor. These differences are detected based on the NIR fluorescence of the dye. Tumors with strong and rapid NIR fluorescence are well vascularized, and tumors or dying tumors with weak and gradual NIR florescence are poorly vascularized, and tumors with a non-fluorescent center contain necrotic tissues lacking vascularization.

Various embodiments of the present invention provide for a method to differentiate live versus dead cells based upon the uptake of organic carbocyanine dyes. The method comprises providing an NIR organic carbocyanine dye, contacting the NIR organic carbocyanine dye to a biological sample; and identifying live cells by observing cellular uptake of the NIR organic carbocyanine dye and/or identifying dead cells by observing the lack of cellular uptake of the NIR organic carbocyanine dye. As the NIR organic carbocyanine dye is actively taken up by live cells, a dead cell would show little or no stain with the NIR organic carbocyanine dye.

Various embodiments of the present invention provide for a method to merge x-ray and fluorescence imaging of a local tumor and its subsequent metastasis. The method comprises: administering an NIR organic carbocyanine dye to a subject; and imaging the subject using an imaging system to obtain an x-ray image and a fluorescence image from the NIR organic carbocyanine dye; and merging the x-ray image and the fluorescence image. In one embodiment, the imaging system may be a single system that can obtain both the x-ray image and the fluorescence image from the NIR organic carbocyanine dye. When administered to the subject, the dye is preferentially taken up by tumor cells, while normal cells have minimal to no uptake. The differential uptake between cancer and normal cells provides a contrast in the dye distribution, which is detected based on the NIR fluorescence of the dye. By comparing the image of NIR and X-ray (which provides anatomical information of the tumor) of the same subject, the locations of the tumor and its metastasis are determined.

Various embodiments of the present invention provide for a method to image formalin or water soluble chemically fixed tissues or frozen sections of tissue specimens for the presence of trace amounts of the NIR organic carbocyanine dyes in tumor cells. The method comprises: providing an NIR organic carbocyanine dye; contacting the NIR organic carbocyanine dye to the fixed tissue or the frozen section of the tissue specimen; and imaging the fixed tissue or the frozen section of the tissue specimen to detect the presence of a tumor cell by detecting the presence of the NIR organic carbocyanine dye. Freshly dissected tissues contain live cells, including live tumor cells. When stained with the NIR organic carbocyanine dye, tumor cells in the freshly dissected tissues will be preferentially stained by the NIR organic carbocyanine dye, because tumor cells can actively uptake the NIR organic carbocyanine dye. The stained tissue will then be fixed and sectioned for detection of the tumor cells and the location of the dye in the context of a tumor cell, based on enhanced NIR fluorescence.

Various embodiments of the present invention provides for a method to conjugate the NIR organic carbocyanine dye with biotin after which the presence of the dye (either by uptake or retention) can be easily detected by the biotin-avidin enzyme conjugate sandwich method in the visible wavelengths. The method comprises providing an NIR organic carbocyanine dye and conjugating the NIR organic carbocyanine dye to biotin.

In various embodiments, the NIR organic carbocyanine dye used in these methods is taken up by the cancer cell or tumor without the need of a chemical conjugation. In various embodiments, the NIR heptamethine cyanine dye used in these methods is taken up by the cancer cell or tumor without the need of a chemical conjugation.

Biological Samples Examples of biological samples include but are not limited to tissue, tumor tissue, cancer tissue, cells, tumor cells, cancer cells, body fluids, whole blood, plasma, stool, intestinal fluids or aspirate, and stomach fluids or aspirate, serum, cerebral spinal fluid (CSF), urine, sweat, saliva, tears, pulmonary secretions, breast aspirate, breast milk, prostate fluid, seminal fluid, cervical scraping, bone marrow aspirate, amniotic fluid, intraocular fluid, mucous, and moisture in breath. In particular embodiments of the method, the biological sample may be whole blood, blood plasma, blood serum or combinations thereof.

In various embodiments, the biological sample is a physiological fluid from the subject. In various embodiments, the physiological fluid may be interstitial fluid, saliva, sweat, urine, whole blood, serum, plasma, cerebral spinal fluid (CSF), bone marrow aspirate, tears, pulmonary secretion, breast aspirate, breast milk, prostate fluid, seminal fluid, amniotic fluid, intraocular fluid, mucous or combinations thereof.

Imaging

In various embodiments, imaging the subject, the cells or the tumor in these methods is performed about 0.5, 1, 2, 3, 4, 5, 6, 7, 8, 9, 10, 11, 12, 13, 14, 15, 16, 17, 18, 19, 20, 21, 22, 23 or 24 hours after administering the NIR organic carbocyanine dye to the subject. In various embodiments, imaging the subject, the cells or the tumor in these methods is performed about 24-48 hours after administering the NIR organic carbocyanine dye to the subject. In another embodiment, imaging the subject, the cells or the tumor in these methods is performed about 48-96 hours after administering the NIR organic carbocyanine dye to the subject. In other embodiments, imaging the subject, the cells or the tumor in these methods is performed about 3, 4, 5, 6, 7, 8, 9, 10, 11, 12, 13, 14, or 15 days after administering the NIR organic carbocyanine dye to the subject.

In various embodiments, imaging the subject, the cells or the tumor in these methods is performed about 0.5, 1, 2, 3, 4, 5, 6, 7, 8, 9, 10, 11, 12, 13, 14, 15, 16, 17, 18, 19, 20, 21, 22, 23 or 24 hours after administering the NIR heptamethine cyanine dye to the subject. In various embodiments, imaging the subject, the cells or the tumor in these methods is performed about 24-48 hours after administering the NIR heptamethine cyanine dye to the subject. In another embodiment, imaging the subject, the cells or the tumor in these methods is performed about 48-96 hours after administering the NIR heptamethine cyanine dye to the subject. In other embodiments, imaging the subject, the cells or the tumor in these methods is performed about 3, 4, 5, 6, 7, 8, 9, 10, 11, 12, 13, 14, or 15 days after administering the NIR heptamethine cyanine dye to the subject.

Cancer and Tumor

In various embodiments, the cancer cell or tumor in these methods is a type selected from the group consisting of local and disseminated human prostate, breast, lung, cervical, skin, renal, leukemia, bladder and osteosarcoma. In particular embodiments, the cancer cell or tumor in these methods is a prostate cancer cell. In particular embodiments, the cancer cell or tumor in these methods is a renal cancer cell.

In various embodiments, the cancer cell or tumor in these methods is a metastasized cancer cell or tumor. In various embodiments, the cancer cell or tumor in these methods is a metastasized prostate cancer cell or tumor. In various embodiments, the cancer cell or tumor in these methods is a metastasized renal cancer cell or tumor.

In various embodiments, the cancer cell or tumor in these methods is a localized or metastatic mouse cancer cell or tumor from a transgenic animal. In various embodiments, the cancer cell or tumor in these methods is a localized or metastatic prostate cancer cell or tumor from a transgenic animal. In various embodiments, the cancer cell or tumor in these methods is a localized or metastatic mouse renal cancer cell or tumor from a transgenic animal. In various embodiments, the tumors identified, detected, imaged, located and/or isolated by these methods are less than 1 mm³.

In various embodiments, as few as 10 cancer cells per milliliter can be identified, detected, imaged, located and/or isolated by these methods. In various embodiments, as few as 9, 8, 7, 6, 5, 4, 3, 2, or 1 cancer cells per milliliter can be identified, detected, imaged, located and/or isolated by these methods. In various embodiments, as few as 1 cancer cell per milliliter can be identified, detected, imaged, located and/or isolated by these methods.

The inventors were able to detect approximately 1.3 cancer cells from a sample comprising 10 cancer cells/ml of human blood. Accordingly, in various embodiments, the method can identify, detect, image, locate and/or isolate at least 1 cancer cell per in 10 cancer cells/ml sample. In various embodiments, the method can identify, detect, image, locate and/or isolate at least 1.3 cancer cells per in 10 cancer cells/ml sample. In various embodiments, the method can identify, detect, image, locate and/or isolate at least 2 or 3 cancer cells per in 10 cancer cells/ml sample. In various embodiments, the method can identify, detect, image, locate and/or isolate at least as 1 cancer cell per 11, 12, 13, 14, 15, 16, 17, 18, 19, or 20 cancer cells/ml sample. This efficiency of detection increased as more the cancer cells were added to the human blood. Accordingly, various embodiments of the present invention encompass the detection of cancer and tumor cells in samples comprising a higher concentration of cancer cells.

In various embodiments, the subject in these methods is a mammalian subject. In various particular embodiments, the subject in these methods is a human subject. In various embodiments, the biological sample in these methods is from a mammalian subject. In various particular embodiments, the biological sample in these methods is from a human subject. In various embodiments, the cancer and/or tumor cells identified, detected, imaged, located, characterized and/or isolated are human cancer and/or tumor cells.

Molecules

In various embodiments, the molecule being detected in these methods is selected from the group consisting of a drug, a radionuclide, a toxin, a substrate, a metabolite, a gene, a gene transcript, a gene modifier, a gene product and combinations thereof.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### The Utility of IR-780 and IR-783 for In Vitro Imaging of Human and Mouse Cells: Molecular Mechanisms of Dye Uptake and Retention

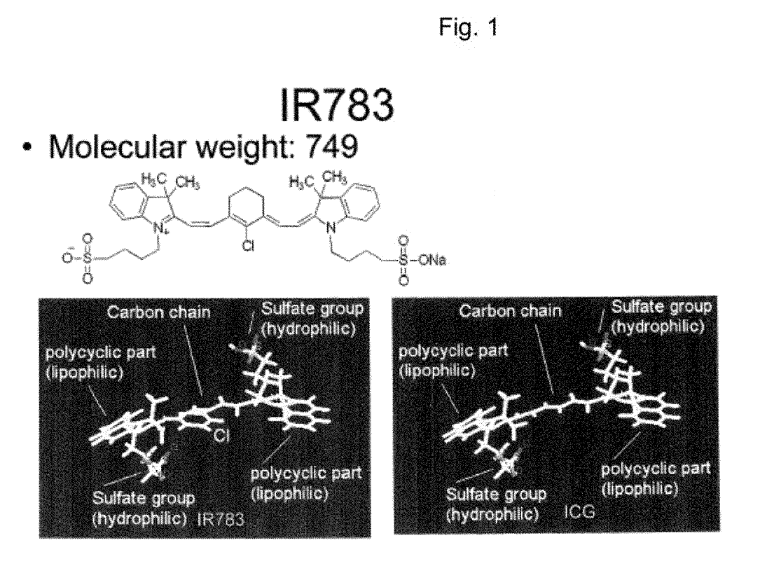

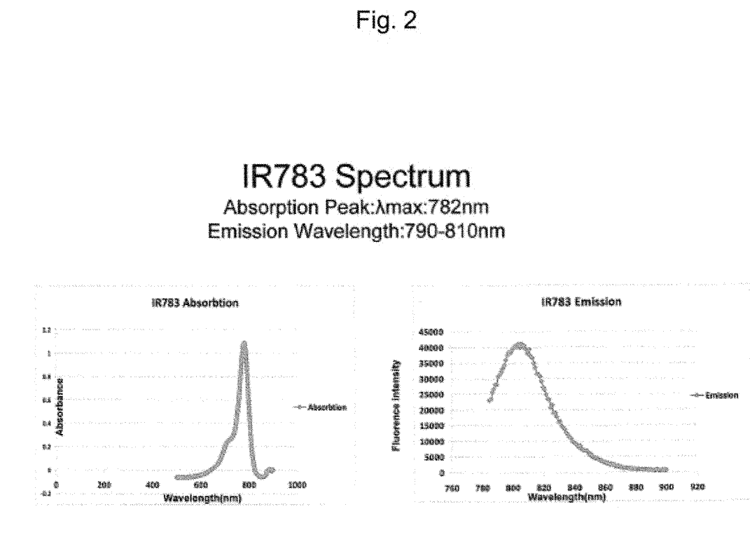

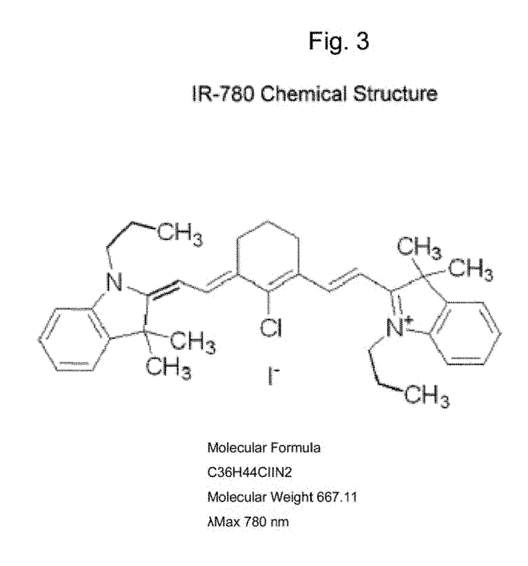

The inventors have evaluated extensively the NIR organic carbocyanine dyes, IR-780 and IR-783 in vitro in a wide range of cultured human and mouse cancer and normal cells. The chemical structures of these dyes and their light absorption and emission are shown FIG. 1-3.

Cancer cells were cultured in T-Medium with 5% FBS at 37° C. incubator. When cells reached confluence of 80-90%, they were trypsinized and plated into 4-well chamber slides with the concentration of 5,000-8000 cells each. The cells were incubated overnight and ready to be used for dye uptake study. The NIR organic dye of interest was dissolved by DMSO at the concentration of 10 mM as the stock solution. The stock solutions were diluted with fresh T-Medium (5% FBS) to the concentration of 50 μM, cells were washed with PBS for 1 min, 2 times at room temperature, and then 800 μL of the diluted dye solution was added into each well of the chamber slides. Cells were incubated with the NIR dye at 37° C. for variable time, then washed with cold PBS for 2 min, 3 times, and then fixed with 10% formalin for 15 min at room temperature. The cells were washed again with PBS for 2 min, 3 times at room temperature, and mounted, covered with VWR cover slides. The chamber slides were observed by using the Zeiss LSM 510 META confocal microscope.

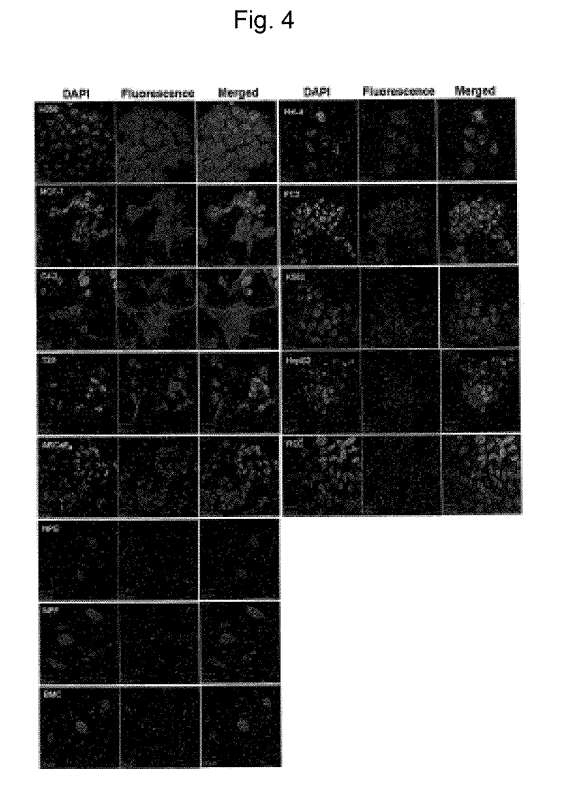

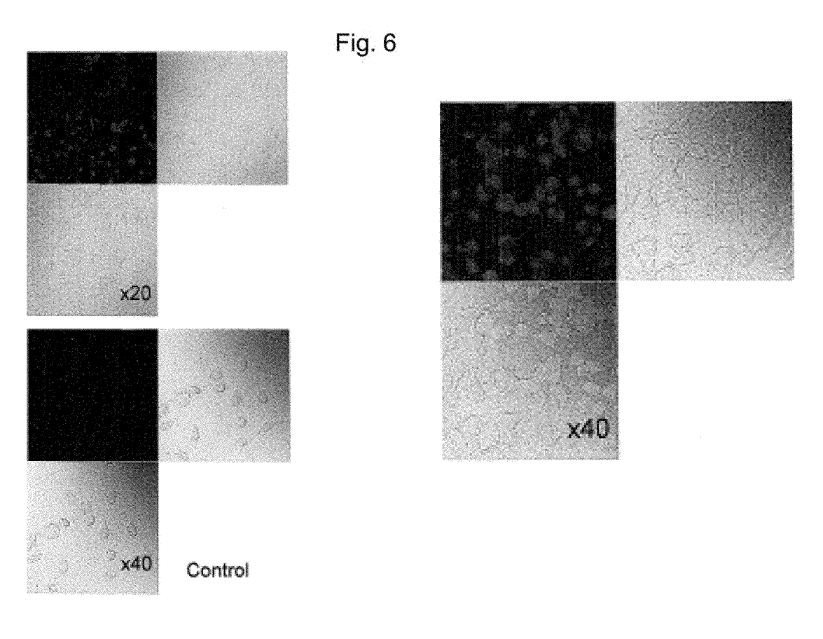

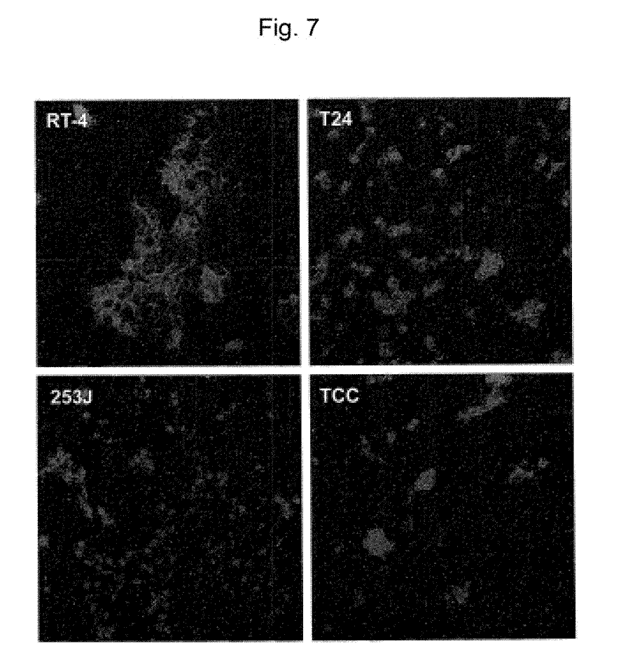

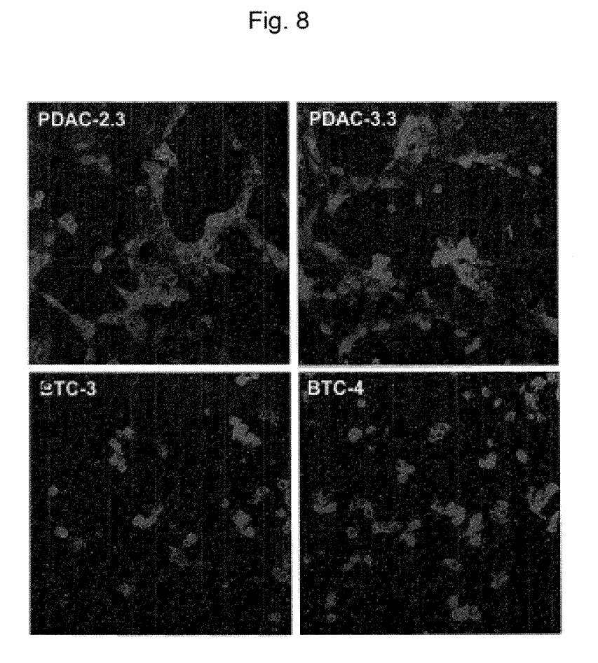

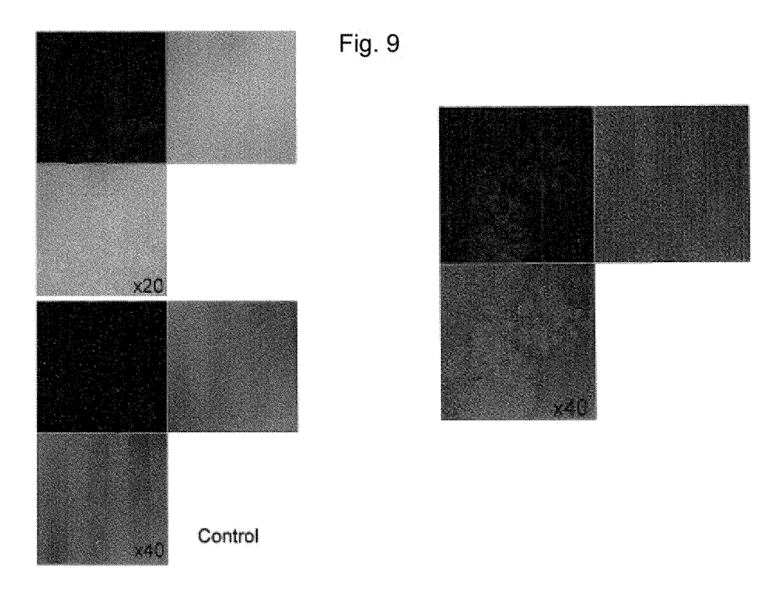

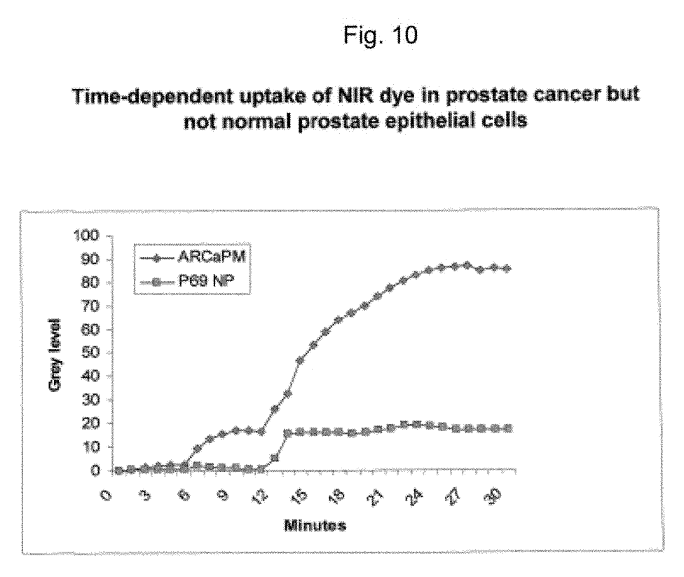

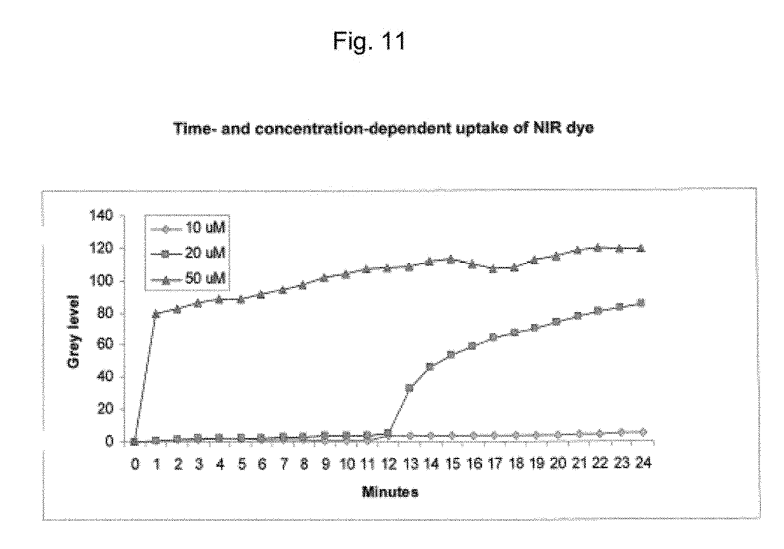

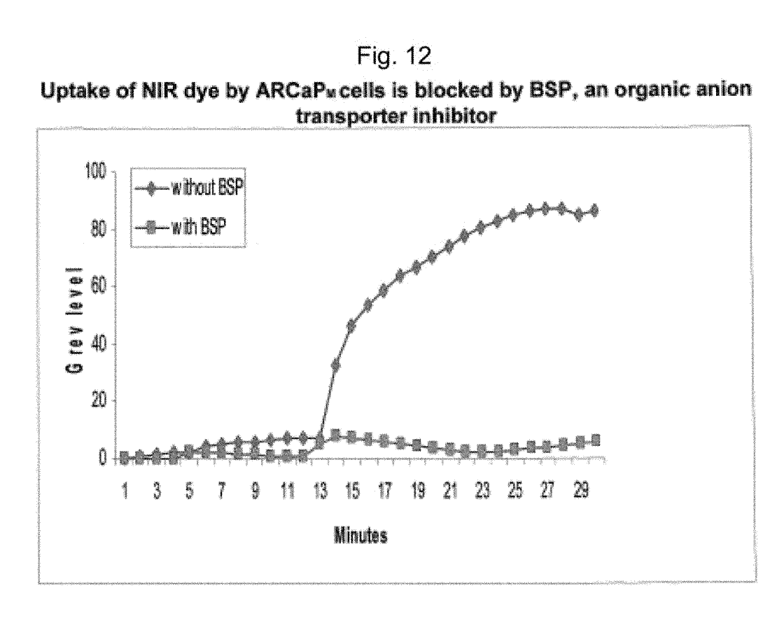

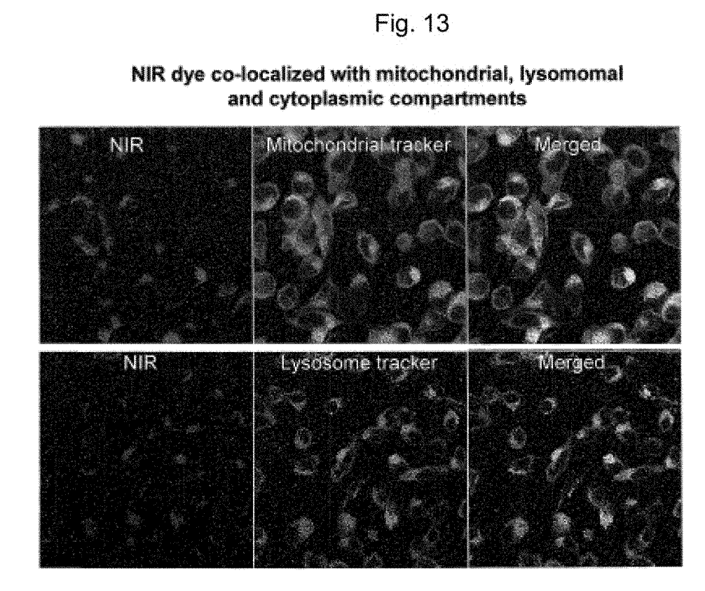

The inventors investigated the uptake of IR-780 and IR-783 by human cancer cell lines grown in culture. As shown in FIG. 4-9, a wide range of cancer cells but not normal cells were taking up these dyes in culture. The uptake of IR-780 by a human prostate cancer (ARCaP_{M}), for example, was compared with normal prostate epithelial cell line (P69) and confirmed that time-dependent uptake differences of IR-780 by human prostate cancer and normal prostate epithelial cells (FIG. 10). The inventors studied the kinetics of this uptake (i.e. time course and concentration dependent) and found that 20-50 μM of IR-780 was needed to measure the uptake of this dye into cancer cells in culture (FIG. 11). The inventors further compared the ability human normal prostate and cancerous epithelial cells to uptake IR-780 in vitro in the absence or presence of an organic anion transporter inhibitor, bromsulphalein (BSP), and found a complete blockade of this dye uptake into human prostate cancer cells by BSP (FIG. 12). Dye tracking studies indicate that once the majority of IR-780 dye enters into prostate cancer cells, it co-localized with both mitochondria and lysosomes (FIG. 13). The uptake of these dyes was found to be an active process since no dye uptake was observed at 0° C. (data not shown).

### Example 2

### The Utility of IR-780 and IR-783 for In Vivo Studies

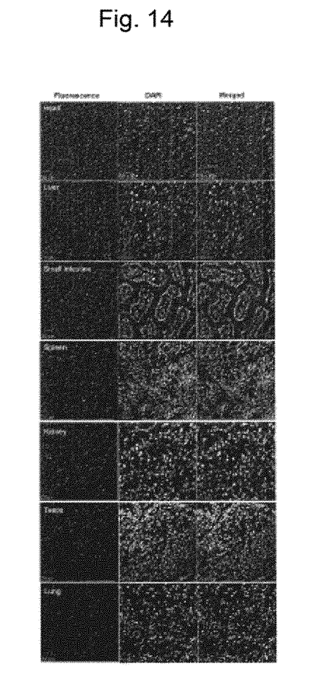

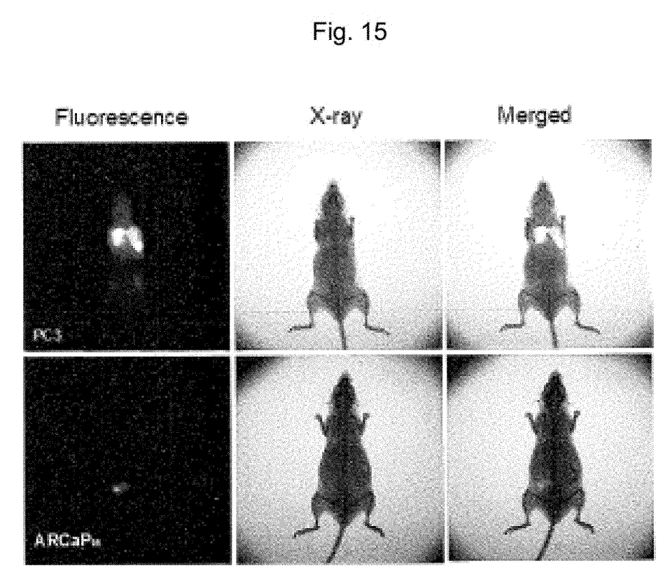

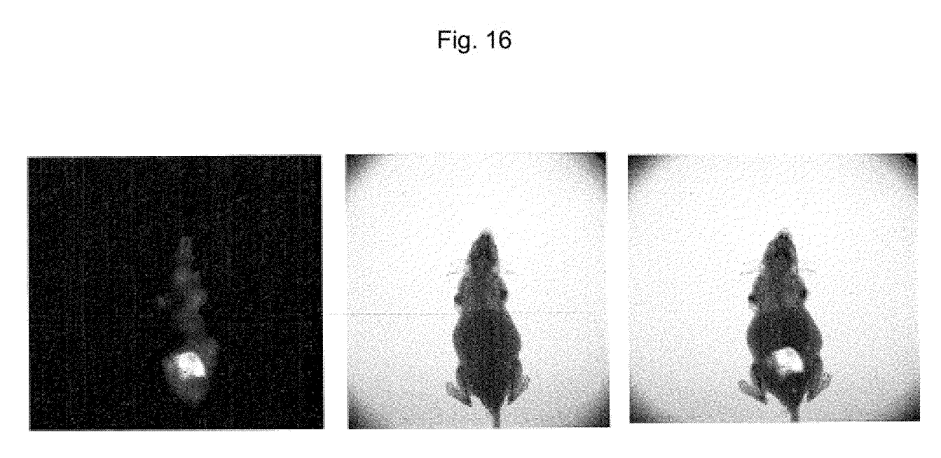

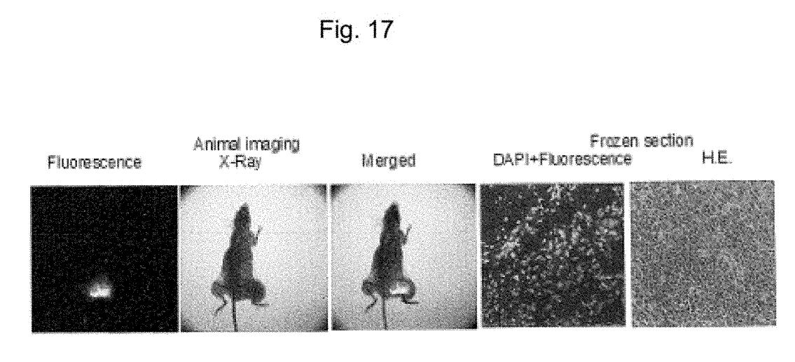

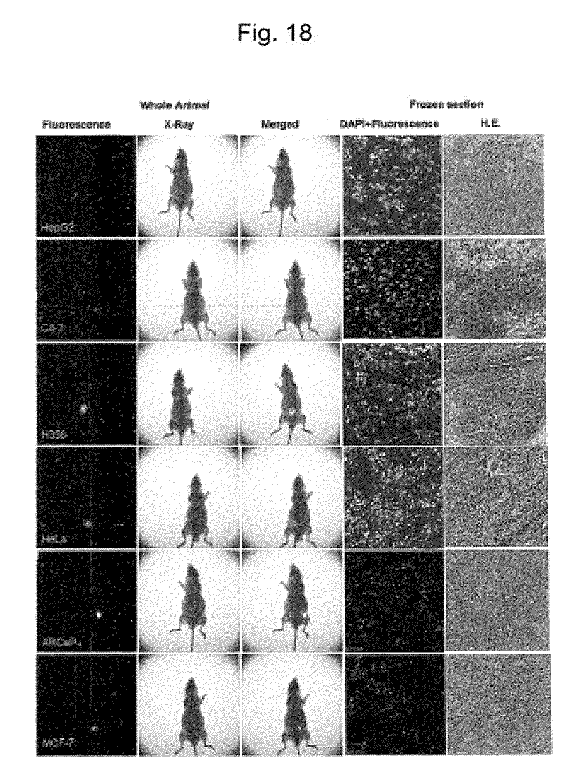

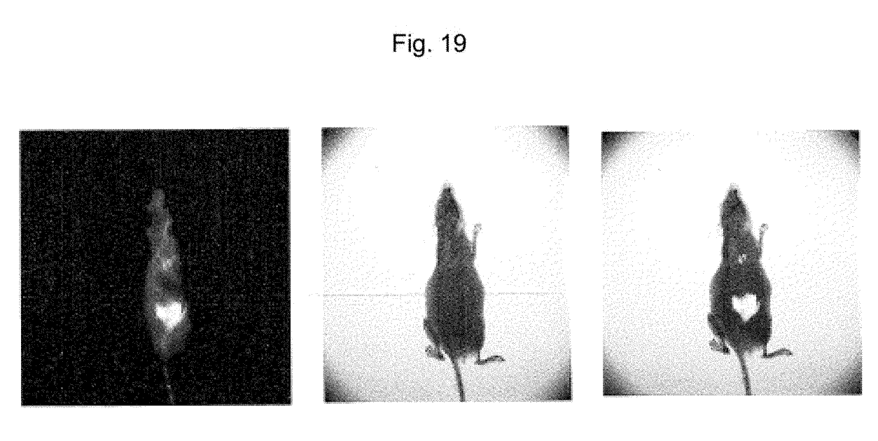

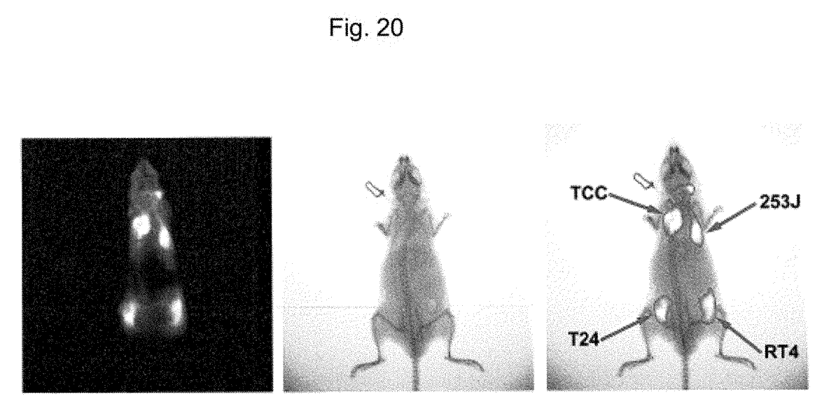

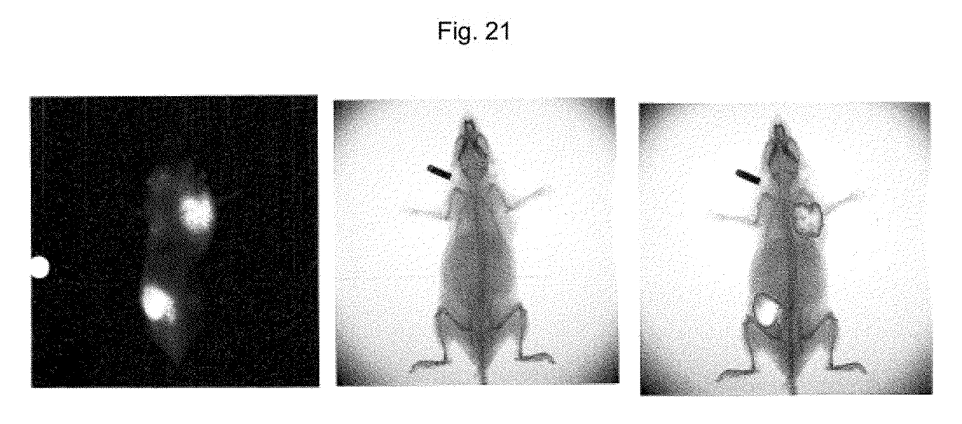

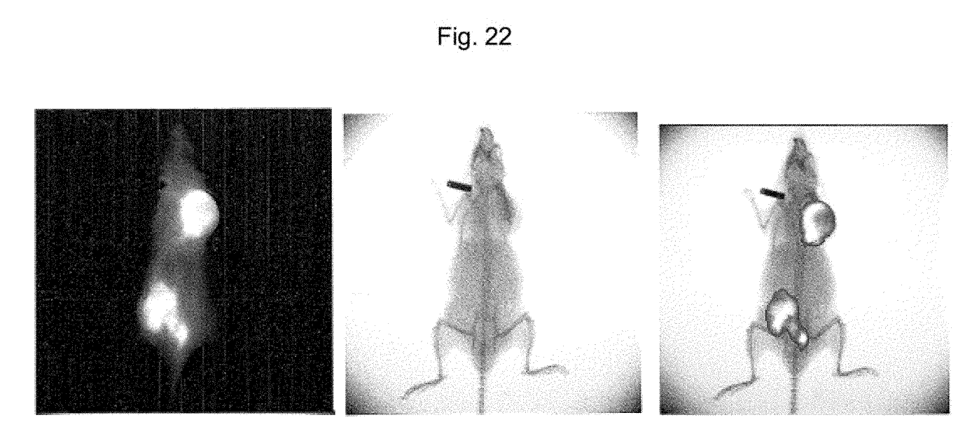

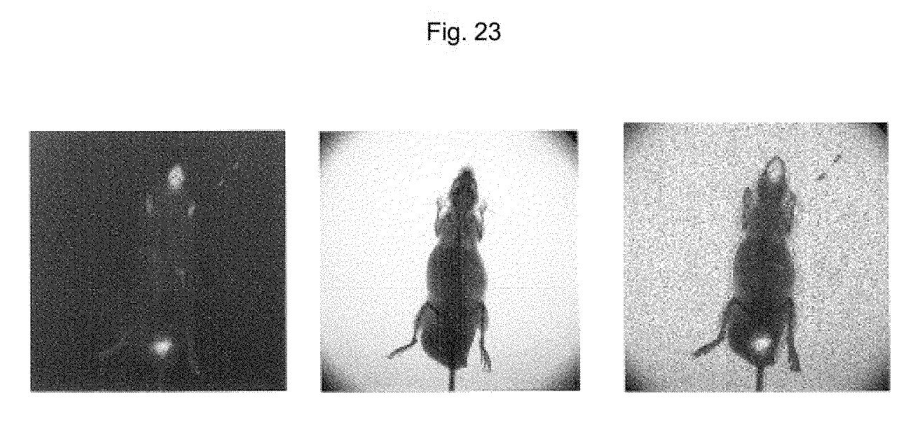

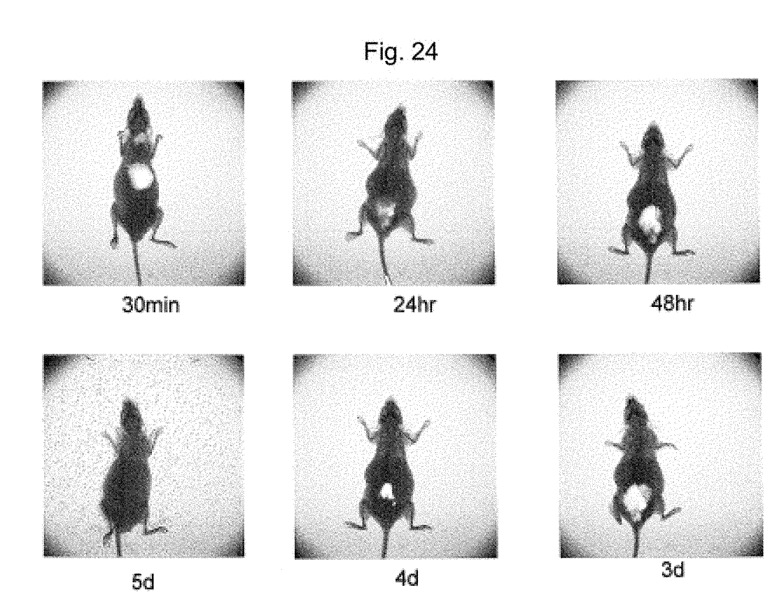

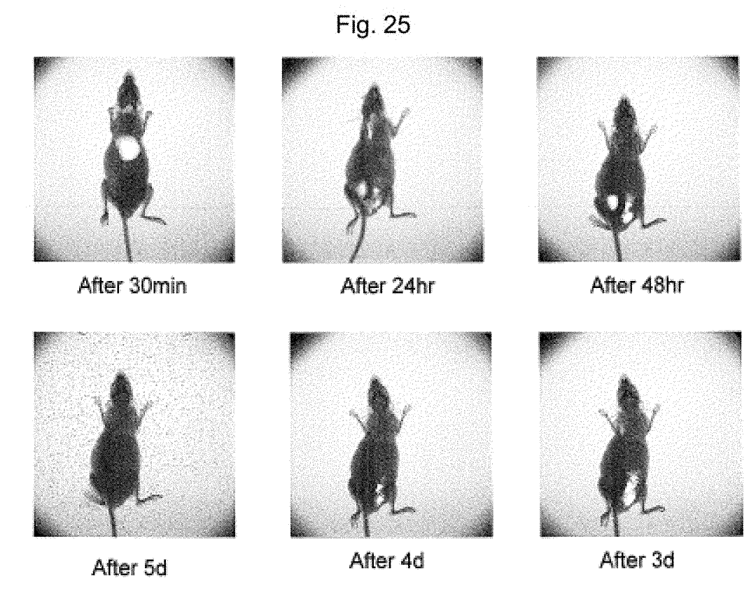

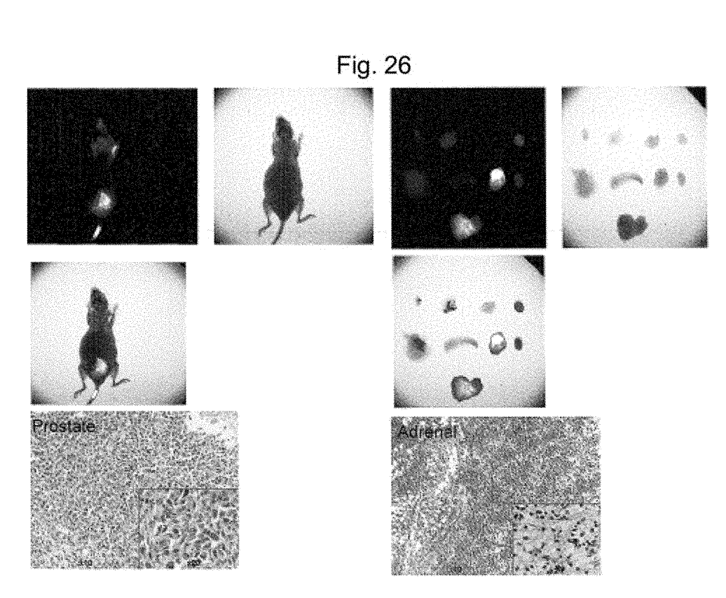

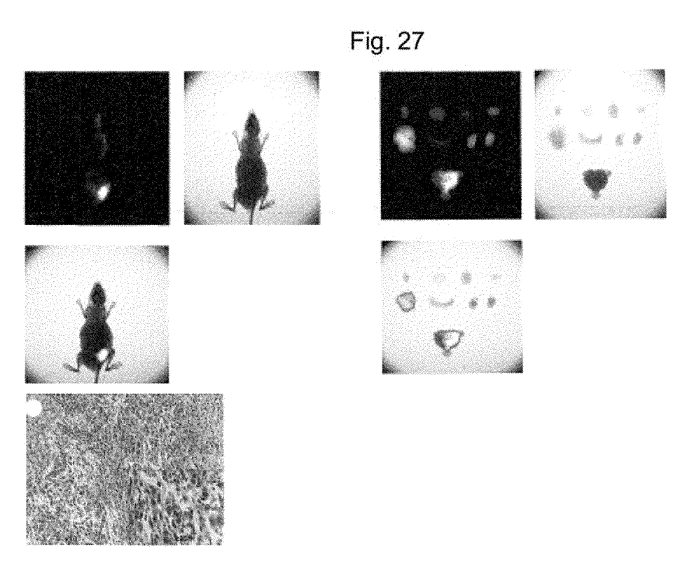

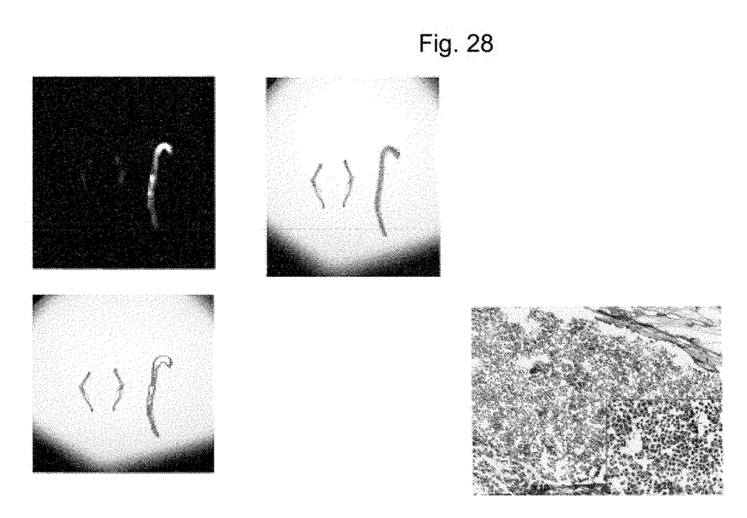

To determine the dye uptake into normal mouse tissues or into tumor xenografts or tumors developed in situ in transgenic mice, NIR dye of interest was injected at a dose of 100 μL at a concentration of 100 μM through tail veins. After variable time of the dye injection, the mice were anesthetized and imaged by using the Kodak animal in vivo imaging system, which can obtain both the NIR signal and the X-ray picture and these images were merged. It was observed that there was no dye uptake into normal mouse tissues (FIG. 14). The uptake of either IR-780 or IR-783 was found in a broad spectrum of human xenograft implants in mice, either grown subcutaneously, orthotopically or intraosseously (FIG. 15-22). IR-783 was also found to accumulate in prostate tumors and their metastases in a transgenic TRAMP mouse, a transgenic mouse strain overexpressed large and small T-antigen in the prostate gland under the control of a prostate-specific probasin promoter (FIG. 23). The time-course of in vivo IR-783 dye uptake studies was conducted and data shown in FIGS. 24 and 25, which indicated that the optimal images may be obtained from metastatic prostate tumors in mice between 24-48 hours. For subcutaneous tumors, it was found that the dye uptake and retention can be as long as 15 days after IR-780 injection (data not shown). IR-783 uptake into metastatic human prostate tumors was studied in ARCaP model. Results showed that this dye is highly effective in localizing tumors cells in soft tissues and in bone (FIG. 26-28).

### Example 3

### The Comparative Aspect of the Toxicities of IR-780 and IR-783 in Inbred Strain of Mice

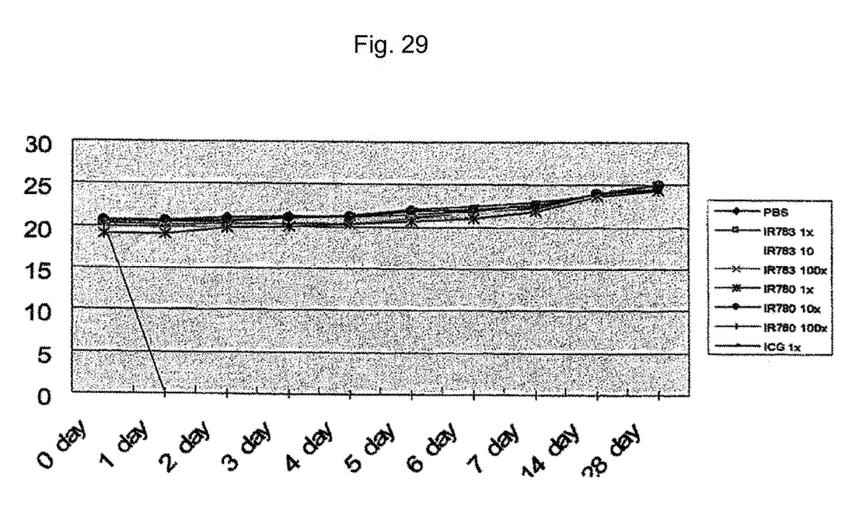

The toxicities of IR-780 and IR-783 in mice was determined by the daily intraperitoneal injection of these dyes at 100× excess of the imaging concentration of these dyes in inbred strain of Balb/c mice. In control mice, the same volume of PBS was injected. The body weight and lethality of these mice were closely followed for 28 days. FIG. 29 showed that while IR-780 appears to be toxic to mice at this elevated dose, IR-783 was found to be completely safe when used at the 100× imaging dose with no lethality in treated mice and all animals gained weight during the period of monitoring with no statistical differences between IR-783-treated and PBS-treated mice. In contrast, IR-780 was found to be highly toxic and killed all mice at day 2 after the injection of this dye at 100× excess of the imaging dose of this dye; no toxicity was detected by injecting mice with indocyanine green (ICG) as evidence by the gains of body weight (see Table 1).

TABLE 1
Toxicity test of dye

| | PBS | IR783 | | | IR780 | | | ICG |
|---|---|---|---|---|---|---|---|---|
| | 1X | 1X | 10X | 100X | 1X | 10X | 100X | 1X |
| 0 day | 20.57 g | 20.29 g | 20.57 g | 19.85 g | 19.00 g | 20.71 g | 20.14 g | 20.43 g |
| 1 day | 20.59 g | 20.20 g | 20.61 g | 19.74 g | 19.02 g | 20.69 g | 0 | 20.49 g |
| 2 day | 21.00 g | 20.23 g | 20.59 g | 20.01 g | 19.71 g | 20.70 g | 0 | 20.51 g |
| 3 day | 21.12 g | 20.32 g | 20.98 g | 20.39 g | 20.01 g | 21.03 g | 0 | 21.01 g |
| 4 day | 21.13 g | 20.34 g | 21.02 g | 20.43 g | 20.32 g | 21.24 g | 0 | 21.43 g |
| 5 day | 21.43 g | 21.00 g | 21.34 g | 21.01 g | 20.56 g | 21.89 g | 0 | 22.00 g |
| 6 day | 22.00 g | 21.87 g | 21.76 g | 21.23 g | 20.97 g | 22.00 g | 0 | 22.34 g |
| 7 day | 22.45 g | 22.32 g | 22.54 g | 22.00 g | 21.81 g | 22.56 g | 0 | 22.96 g |
| 14 day | 23.98 g | 23.91 g | 24.01 g | 23.76 g | 23.56 g | 24.00 g | 0 | 23.87 g |
| 28 day | 25.05 g | 24.86 g | 24.87 g | 24.61 g | 24.44 g | 24.56 g | 0 | 25.00 g |

### Example 4

### IR-783 as an Imaging Agent for the Detection of Cancer Cells in Freshly Harvested Human Renal and Bladder Cancer Specimens

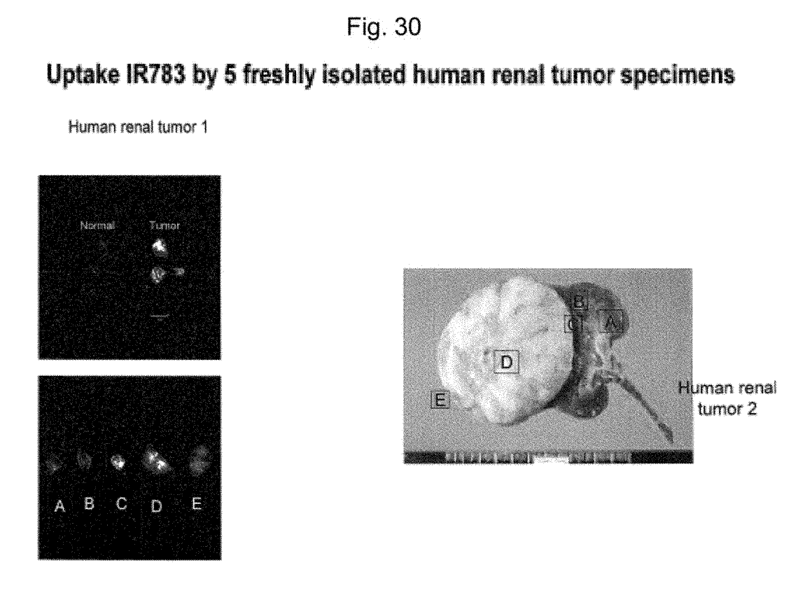

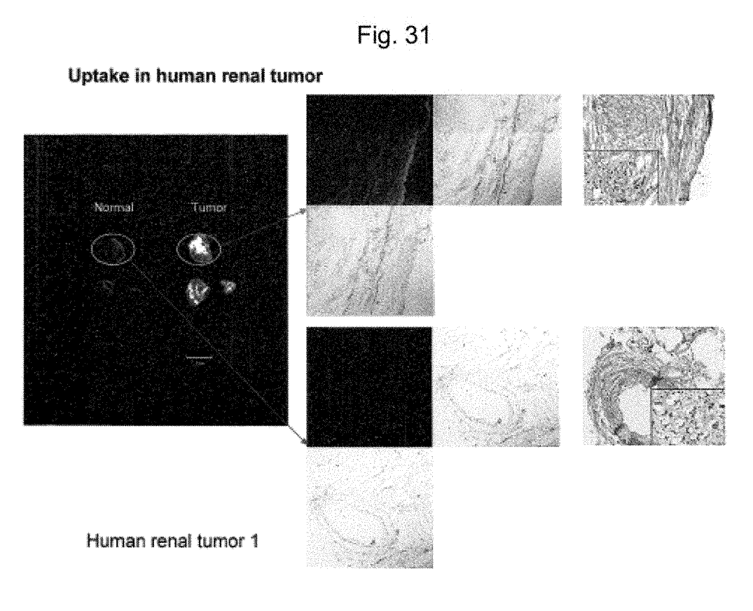

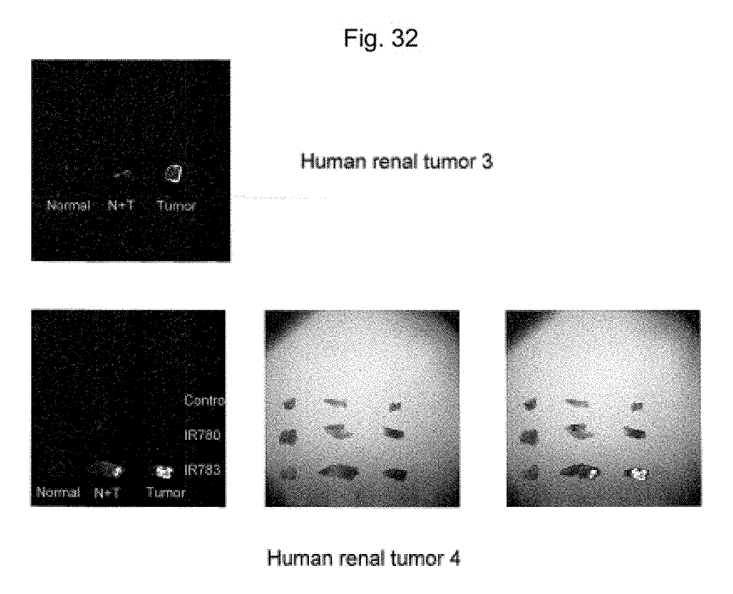

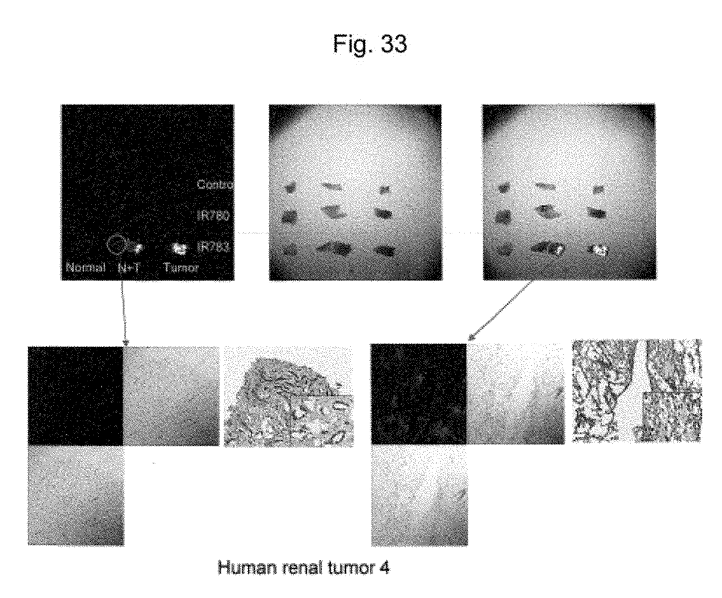

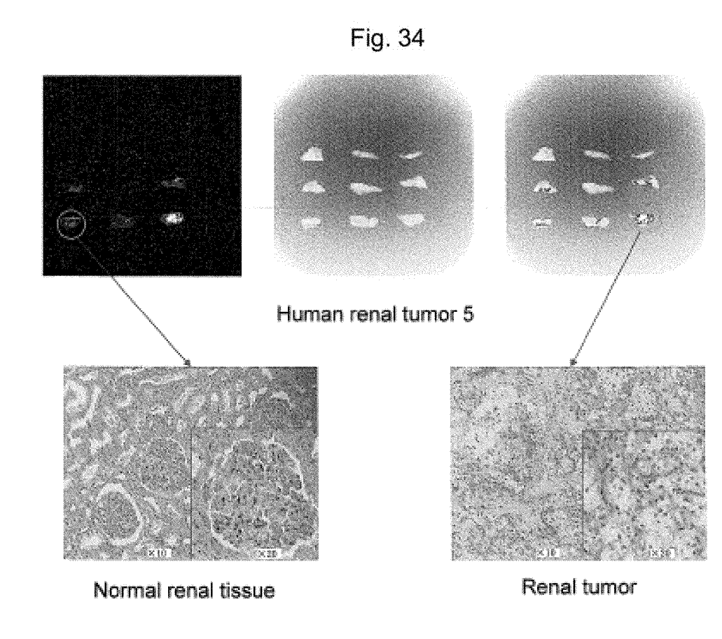

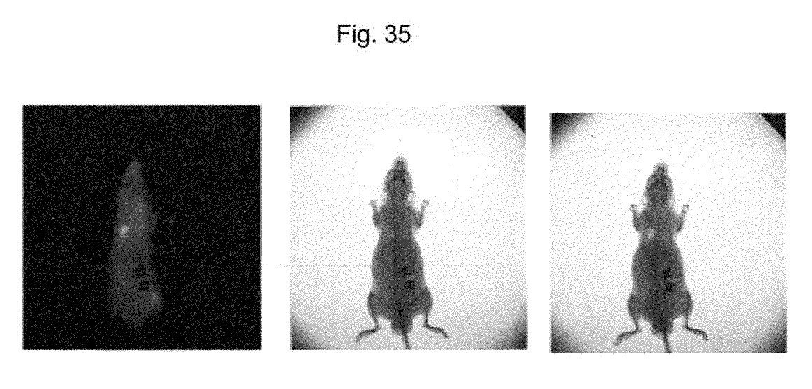

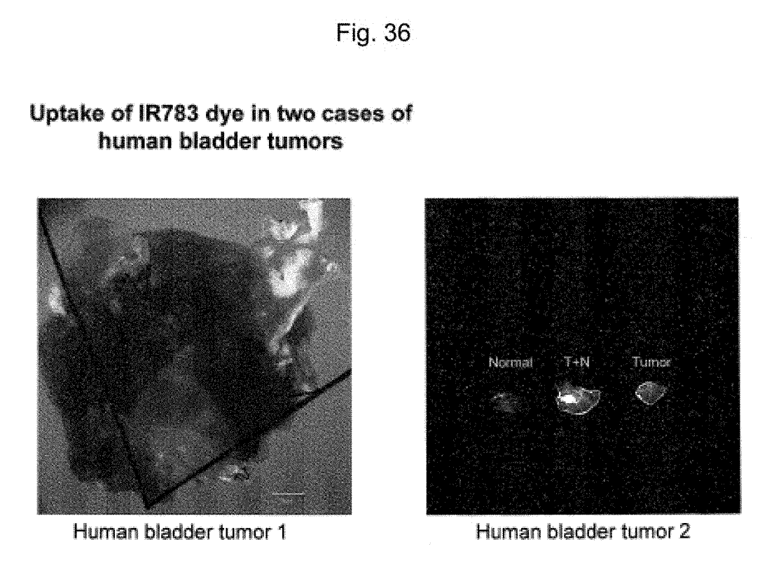

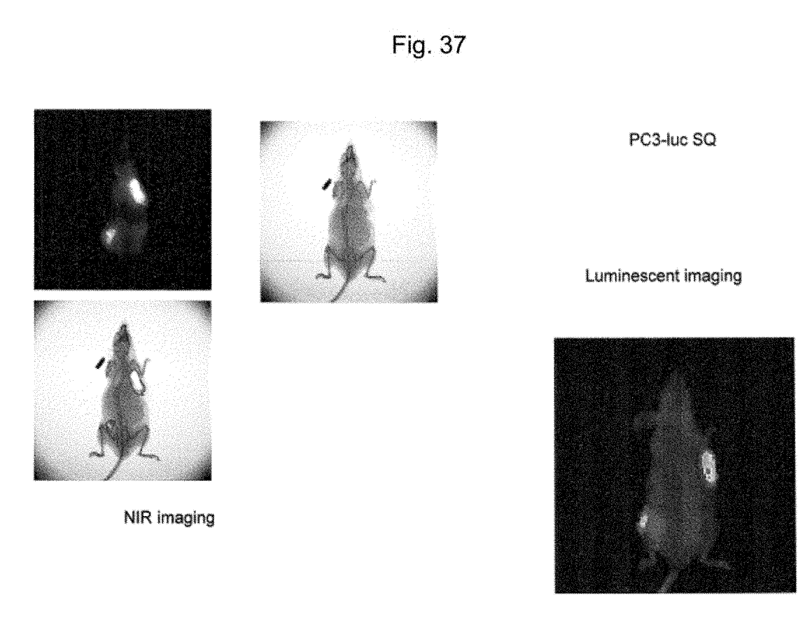

The inventors determined the ability of IR-783 as an imaging agent for freshly harvested human renal and bladder cancer specimens. Results indicate that this dye can be taken up into freshly obtained human renal (FIG. 30-34) and bladder (FIG. 36) specimens, but with substantial low uptake into normal adjacent tissues. Fresh tumor xenografts grown in mice (FIG. 35) are able to uptake the dye. These results were all confirmed by confocal analyses of the tumor and normal tissue specimens (see above Figures). In these studies, interestingly, the inventors observed that this dye can also be taken up by fatty tissues. The nature of this uptake at this time is not clear. IR-783 dye uptake into tumor tissue xenografts is superior to luminescence imaging since the former technique also provide valuable anatomical information of the tumors (FIG. 37).

Since the dyes can be taken up by cultured human cancer cell lines and freshly isolated human tissues, the inventors believe that this dye can be uptaked into cancer cells in circulating body fluids such as serum, blood, saliva, bone marrow, milk and in urine.

### Example 5

### The Usage of Carbocyanine Dyes in Personalized Oncology and Medicine

It is disclosed herein that IR-783, an organic carbocyanine dye with minimal host toxicity, can be used effectively for imaging of a broad spectrum of human tumor cells and solid tumors in live mice. This compound and other related carbocyanine dye analogues, was found to be highly effective as well (e.g., IR-780, MHI-148) and they all appear to be unable to be taken up by normal cells in vitro. Although this organic dye can be taken up transiently by some normal mouse tissues, such as liver, kidney, testis and seminal vesicles, this dye was observed to be cleared from these organs within a 96-hr time period in live animals. Because of the attractive pharmacokinetic properties of this compound (excreted or metabolized by host mice but accumulated in tumors when examined at 48-96 hrs after IR-783 administration), its near-infrared fluorescence emission with no autofluorescence from mouse hair, skin and internal organs making this dye suitable for both surface (subcutaneous) and deep (intratibia) tumor imaging. Because of its high intensity due to a high extinction coefficient of this dye, IR-783 can be conveniently used to image and will give unbiased signals from mice without host autofluorescence contamination.

IR-783 was also found to be relatively photostable, and can be used to image tumors approximately 1 mm³ or 1 mg of tumor weight in mice.

IR-783 can be conjugated to cytotoxic drugs, radionuclide, other organic or inorganic molecules targeting tumor, but not normal cells. Uptake of IR-783 either alone or with therapeutic moieties can be accomplished in cancer cells without the need of the attachment of a targeting ligand. Based upon the unique and attractive pharmacoltinetic properties of IR-783 (i.e. washout from systemic circulation but retained in tumors), the inventors believe that this compound can be chemically conjugated to both organic, inorganic molecules, radioactive chemicals or other pharmaceuticals for the imaging and treatment of metastatic tumor growth in live animals and humans for a number of attractive applications. For example, clinical and preclinical applications of IR-783 include but not limited to the following. (1) To study the in situ pharmacokinetics and pharmacodynamics of pharmaceuticals in live animals and humans. This application is important and significant because, pharmacokinetics and pharmacodynamics are measured using bodily fluids available for detection. The ability of IR-783 to be specifically taken up by tumor tissues and its conjugation to drugs could lead to the development of novel methods of determining the in situ pharmacokinetics and pharmacodynamics of clinically useful drugs in the body and at tumor sites, a task that cannot be achieved with current technology. (2) IR-783 can be used as a chemical tag for tumor cells. The tagged tumor cells can be isolated by FACS sorting. The isolated tumor cells from biological fluids can be further characterized. IR-783 can also be used to tag stem cells, putative tumor stem cells or any biologicals that can penetrate organ, tissue or cellular compartment for improved real-time imaging. (3) IR-783 can be chemically conjugated to drugs, organic and inorganic molecules, or radionuclides (also referred to as IR-783 conjugates) without the need of chemical conjugation with cell specific ligands for both cancer imaging and targeting. Because of its unique property, IR-783 conjugates can be visualized at the site of interaction with cancer tissues and cells and such interactions can be quantitatively and qualitatively assessed. (4) IR-783 can be used as an agent to image and treat patients on a personalized basis. It is known that drug accumulation and metabolism in individual patients are different, but unfortunately it is difficult to monitor the pharmacokinetic and pharmacodynamic properties of drugs with desirable precision. IR-783 conjugates can be assessed directly at the tumor sites, thus providing vital and novel information on the pharmacokinetic and pharmacodynamic properties of the drugs, which is currently only possible by assessing these parameters in serum. (5) IR-783 can be used to tag circulating stem cells for the assessment of stem cell grafting of bone marrow or other vital organs. Normal cells may have a low rate of uptake and this can be improved by the use of internalized cell surface ligand in conjugation with IR-783, which could potentially increase the quantity of dye uptake into stem cell populations for trafficking. (6) This same concept can be applied by the conjugation of IR-783 to other tissue and organ-specific cell surface molecules for potential imaging and targeting purposes. For example, IR-783 can be guided into human benign hyperplastic prostate cells and tissues or pre-malignant and malignant cells using a prostate cell surface ligand plus cytotoxic drug conjugates for the non-surgical ablation of benign, pre-malignant and malignant cells.

### Example 6

### Chemicals

The heptamethine cyanine dyes IR 783 (2-[2-[2-Chloro-3-[2-[1,3-dihydro-3,3-dimethyl-1-(4-sulfobutyl)-2H-indol-2-ylidene]-ethylidene]-1-cyclohexen-1-yl]-ethenyl]-3,3-dimethyl-1-(4-sulfobutyl)-3H-indolium) and IR-780: 2-[2-[2-Chloro-3-[(1,3-dihydro-3,3-dimethyl-1-propyl-2H-indol-2-ylidene)ethylidene]-1-cyclohexen-1-yl]ethenyl]-3,3-dimethyl-1-propylindolium iodide) were purchased from Sigma-Aldrich (St. Louis, Mo.) and purified by the published methods^{67,68}.

The heptamethine cyanine dye MHI-148, (2-[2-[2-Chloro-3-[2-[l,3-dihydro-3,3-dimethyl-1-(5-carboxypentyl)-2H-indol-2-ylidene]-ethylidene]-1-cyclohexen-1-yl]-ethenyl]-3,3-dimethyl-1-(5-carboxypentyl)-3H-indolium bromide), was synthesized and purified as described previously (16-18). The other heptamethine cyanine dyes and their derivatives (see Table 2) were also prepared by published procedures.

All materials were dissolved in DMSO diluted with appropriate vehicles, filtered through 0.2 μm filters and stored at 4° C. before use.

TABLE 2
Active and inactive heptamethine cyanine dyes

| Active days^{a} | Inactive dyes^{b} |
|---|---|
| 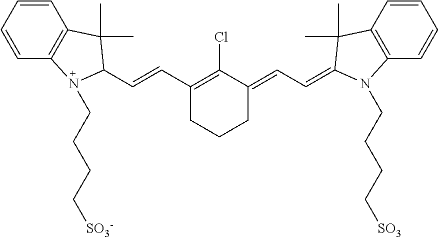 IR-783 | 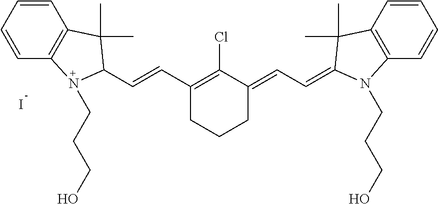 IR-1 |
| 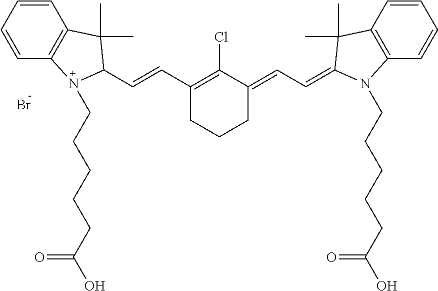 [SMILES: CC1(C)/C(=C\C=C2/CCCC(/C=C/C3N(CCCCCC(=O)O)c4ccccc4C3(C)C)=C2Cl)N(CCCCCC(=O)O)c2ccccc21 ; Br] MHI-148 | 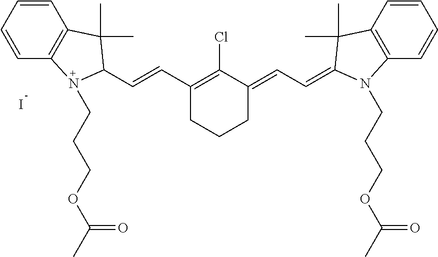 IR-2 |
| | 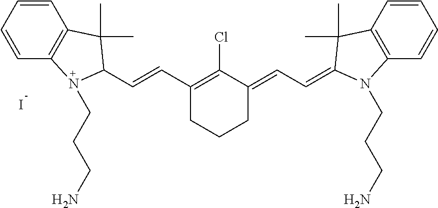 IR-3 |
| | 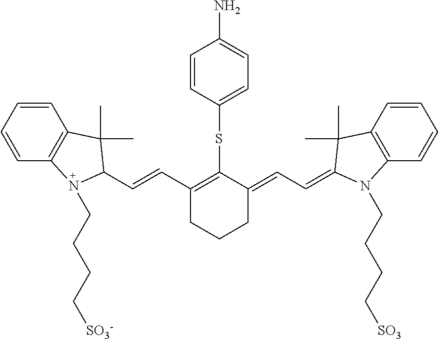 IR-4 |

^{a}Activity of the dyes denotes specific uptake by cultured human cancer cell lines in vitro and their xenograft tumors in vivo, setermined by NIR fluorescence imaging.
^{b}These heptamethine cyanine dyes were not accumulated in tumor cells in vitro and xenograft tumors in vivo.

### Example 7

### Cell Lines and Cell Culture

Human cancer cells used in this study were: prostate cancer (LNCaP, C4-2, C4-2B, ARCaP_{E}, ARCaP_{M}, and PC-3), lung cancer (H358), breast cancer (MCF-7), cervical cancer (HeLa), leukemia (K562), renal cancer (SN12C, ACHN), bladder cancer (T24) and pancreatic cancer (MIA PaCa-2). As controls, normal human bone marrow stroma cells (HS-27A), normal human prostate epithelial cells (P69 and NPE), normal human prostate fibroblasts (NPF), human vascular endothelial cells (HUVEC-CS), and human embryonic kidney cells (HEK293) were used. LNCaP, ARCaP, and their lineage-derived cells (C4-2 and C4-2 B) were established by our laboratory and cultured in T-medium as described (19, 20). Human prostate epithelial cells, NPE, and human prostate fibroblasts, NPF, were derived from the normal areas of prostatectomy specimens by our laboratory using an Emory University approved protocol and were maintained in T-medium as described (21). SN12C was obtained from a patient with renal clear cell carcinoma (22) and was cultured in T-medium. Unless otherwise specified, all of the other human cell lines were purchased from American Type Culture Collection (ATCC, Manassas, Va.) and were cultured in ATCC recommended media, with 5% fetal bovine serum (FBS) and 1× penicillin/streptomycin at 37° C. with 5% CO₂. Also investigated in this study was the dye uptake by mouse pancreatic cancer cell lines, PDAC2.3, PDAC3.3, BTC3 and BTC4, derived from transgenic mice, kindly provided to us by Dr. Douglas Hanahan from University of California at San Francisco. These cells were also cultured in T-medium.

### Example 8

### Cell and Tissue Uptake Study Using NIR Heptamethine Cyanine Dyes

Cells (1×10⁴/well) were seeded on vitronectin-coated four-well chamber slides (Nalgen Nunc, Naperville, Ill.) and incubated with T-medium containing 5% FBS for 24 h. After the cells had attached to the chamber slides, the cells were washed with PBS (Phosphate Buffered Saline) and exposed to the cyanine dye at a concentration of 20 μM in T-medium. The slides were incubated at 37° C. for 30 min, washed twice with PBS to remove excess dyes, and cells were fixed with 10% formaldehyde at 4° C. The slides were then washed twice with PBS and covered with glass coverslips with an aqueous mounting medium (Sigma-Aldrich, St. Louis, Mo.). Images were recorded by confocal laser microscopy (Zeiss LSM 510 META, Germany) using a 633 nm excitation laser and 670-810 nm long pass filter, or a fluorescence microscope (Olympus 1×71; Olympus, Melville, N.Y.) equipped with a 75 W Xenon lamp and an Indo-Cyanine Green filter cube (excitation 750-800 nm; emission 820-860 nm) (Chroma, Rockingham, Vt.).

To determine dye uptake in tissues, tissues isolated from tumor bearing mice (see below) were placed in OTC medium and frozen at −80° C. Frozen 5 μm tissue sections were prepared for histopathologic observation using the microscope as described above.

### Example 9

### Assessment of Cyanine Dye Uptake into the Mitochondria and Lysosomes of Cancer Cells

Cells were plated on live-cell imaging chambers (World Precision Instrument, Sarasota, Fla.) overnight. Cells were exposed to cyanine dyes at different concentrations and dye uptake was evaluated by a Perkin-Elmer Ultraview ERS spinning disc confocal microscope. This system was mounted on a Zeiss Axiovert 200m inverted microscope equipped with a 37° C. stage warmer, incubator, and constant CO₂ perfusion. A 63× or 100× Zeiss oil objective (numerical aperture, 1.4) was used for live cell images and a Z-stack was created using the attached piezoelectric z-stepper motor. The 633 nm laser line of an argon ion laser (set at 60% power) was used to excite the cyanine dyes. Light emission at 650 nm, while not optimal for these dyes, was detected and was found to correlate directly with the dye concentrations in the cells (FIG. 44). For comparative studies, the exposure time and laser intensity were kept identical for accurate intensity measurements. Pixel intensity was quantified using Metamorph 6.1 (Universal Imaging, Downingtown, Pa.) and the mean pixel intensity was generated as grey level using the Region Statistics feature on the software (23). To determine the dye uptake by the mitochondria, the mitochondrial tracking dye Mito Tracker Orange CMTMROS (Molecular Probes, Carlsbad, Calif.) was used. To determine the dye localization in lysosomes, a lysosome tracking dye, Lyso Tracker Green DND-26 (Molecular Probes, Carlsbad, Calif.), was selected. Imaging of mitochondrial and/or lysosome localization of the cyanine dye was conducted under confocal microscopy (24).

To determine if the cyanine dye uptake and accumulation in cancer cells was dependent upon OATPs, cells were preincubated with 250 μM bromosulfophthalein (BSP), a competitive inhibitor of organic anion transporting peptides (OATPs) (25), for 5 minutes prior to incubating the cells with cyanine dyes. The uptake and accumulation of cyanine dyes in the presence and absence of BSP were conducted in the stage warmer incubator for a period of 35 minutes. The levels of cyanine dye taken up and accumulated in normal prostate (P69) and prostate cancer (ARCaP_{M}) cells were determined and compared on a real-time basis.

### Example 10

Uptake and accumulation of cyanine dyes in tumors in live mice Human cancer cells were implanted (1×10⁶) either subcutaneously, orthotopically, or intraosseously into 4 to 6 week old athymic nude mice (National Cancer Institute, Frederick, Md.) according to our previously published procedures (26, 27). All animal studies were conducted under the Emory University Animal Care and Use Committee guidelines. When tumor sizes reached between 1-6 mm in diameter, as assessed by X-ray or by palpation, mice were injected i.v. or i.p. with cyanine dyes at a dose of 0.375 mg/kg or 10 nmol/20 g mouse body weight. Whole body optical imaging was taken at 24 h using a Kodak Imaging Station 4000 MM (New Haven, Conn.) equipped with fluorescent filter sets (excitation/emission, 800/850 nm). The field of view (FOV) was 120 mm in diameter. The frequency rate for NIR excitation light was 2 mW/cm². The camera settings included maximal gain, 2×2 binning, 1024×1024 pixel resolution, and an exposure time of 5 sec. In some instances, live mice were also imaged by an Olympus OV100 Whole Mouse Imaging System (excitation 762 nm; emission 800 nm) (Olympus Corp., Tokyo, Japan), containing a MT-20 light source (Olympus Biosystems, Planegg, Germany) and DP70 CCD camera (Olympus). Prior to imaging, mice were anesthetized with ketamine (75 mg/kg). During imaging, mice were maintained in an anesthetized state.

The spontaneous metastasis of ARCaP_{M} tumor cells stably transduced with an AsRed2 red fluorescence protein (RFP) (Clontech, Mountain View, Calif.) by injecting these cells orthotopically in mice was studied. ARCaP_{M}-RFP metastasis was determined by the same procedures described above for capturing cyanine dye tumor imaging after IR-783 i.p. injection at a dose of 10 nmol/20 g. In addition, at the time of sacrifice both frozen and paraffin embedded tissue sections were obtained for RFP and confocal fluorescence imaging. Positive identification of ARCaP_{M}-RFP cells was accomplished by fluorescence microscopy and validated by subculturing ARCaP_{M}-RFP cells directly from bone metastasis tissue specimens.

The uptake of cyanine dyes by the TRAMP mouse prostate model and the Apc^{Min/+} mouse-adenoma model (obtained from The Jackson Laboratory, Bar Harbor, Me.) was assessed by a similar protocol as described above. We also utilized the Olympus OV100 imaging system to detect adenoma in the Apc^{Min/+} mouse model. In brief, mice were injected intraperitoneally with IR-783 dye at a dose of 10 nmol/20 g body weight and animals were subjected to total body cyanine dye imaging as described above. Animals were sacrificed at 48 hrs after dye administration and tumors were dissected and subjected to NIR imaging. The presence of tumor cells in tissue specimens was confirmed by histopathologic analysis.

### Example 11

### Assessments of Heptamethine Cyanine Dye Biodistribution in Normal and Tumor-Bearing Mice In Vivo

To assess tissue distribution of these dyes, athymic mice without tumor implantation were sacrificed at 0, 6 and 80 hrs (N=3 each) after i.v. injection of IR-783 dye at a dose of 10 nmol/20 g. Dissected organs were subjected to NIR imaging by a Kodak Imaging Station 4000 MM. In another study the mice bearing orthotopic ARCaP_{M} tumors were subjected to NIR imaging at 0.5, 24, 48, 72 and 96 hrs after IR-783 i.v. administration at a dose of 10 nmol/20 g. In some cases, we also assessed the biodistribution of NIR dye by a spectral method in tissues harvested from athymic mice bearing subcutaneous ARCaP_{M} tumors (N=6). Tumors and normal host organs were homogenized in PBS, centrifuged at 15,000×g for 15 minutes to recover the supernatant fraction after the mice were injected i.p. with IR-783 at a dose of 10 nmol/20 g. The presence of the organic dyes (parental IR-783 and its metabolites) in tissues was estimated spectrophotometrically at an emission wavelength of 820 nm by a PTI Near Infrared Fluorometer QuantaMaster™ 50 (PTI, Birmingham, N.J.) equipped with a 75-watt xenon arc lamp under 500 to 1700 nm InGaAs detector using known concentrations of IR-783 as the standard (28). In other cases, tumor tissues harvested from mice were stored in formalin from 1 week to 3 months, and fluorescence images were obtained and compared.

### Example 12

### Detection of Cancer Cells in Human Blood

An experimental model of evaluating human prostate cancer cells in blood was developed. In brief, heparinized whole blood from human volunteers was collected according to an Emory University approved IRB protocol. A known number of human prostate cancer cells (10-1,000) were added to 1 ml of whole blood, mixed gently with 20 μM IR-783 and incubated for 30 minutes at 37° C. The mononuclear cells and cancer cells were recovered by gradient centrifugation using Histopaque-1077 (Sigma, St. Louis, Mo.). The isolated live cells were observed under a confocal fluorescence microscope.

### Example 13

### Assessment of Systemic Toxicity of IR-783 in Mice

The systemic toxicity of IR-783 was investigated in C57BL/6 mice (National Cancer Institute, Frederick, Md.) by injecting the dye by an i.p. route. The mice (N=8 per group) were subdivided into 4 groups and received PBS as control and IR-783 i.p. injection daily at the following doses: 0.375 mg/kg (imaging dose), 3.75 mg/kg and 37.5 mg/kg. They were weighed daily and their physical activities were observed for one month following dye injection. The histomorphologic appearance of their vital organs was assessed at the time of sacrifice.

### Example 14

### Data Processing and Statistics

The statistical significance of all data was determined by Student's t-test. Data were expressed as the average±standard error of the mean of the indicated number of determinations. The statistical significant difference was assigned as P<0.05.

### Example 15

### Structural Requirement of Heptamethine Cyanine Dyes for Tumor-Specific Uptake and Retention

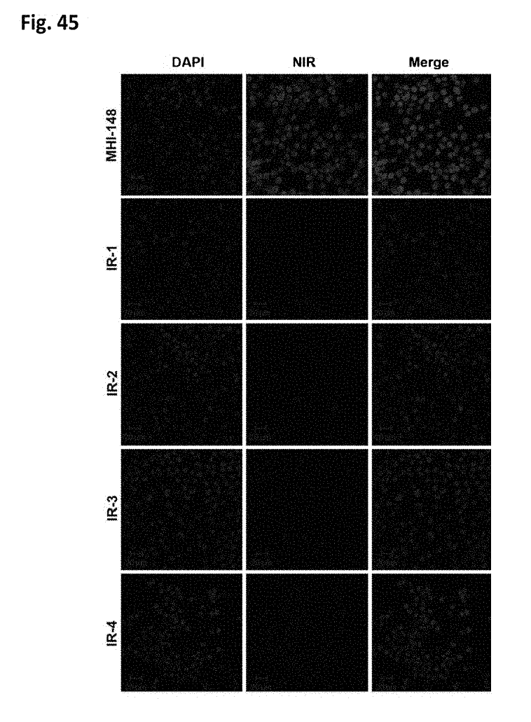

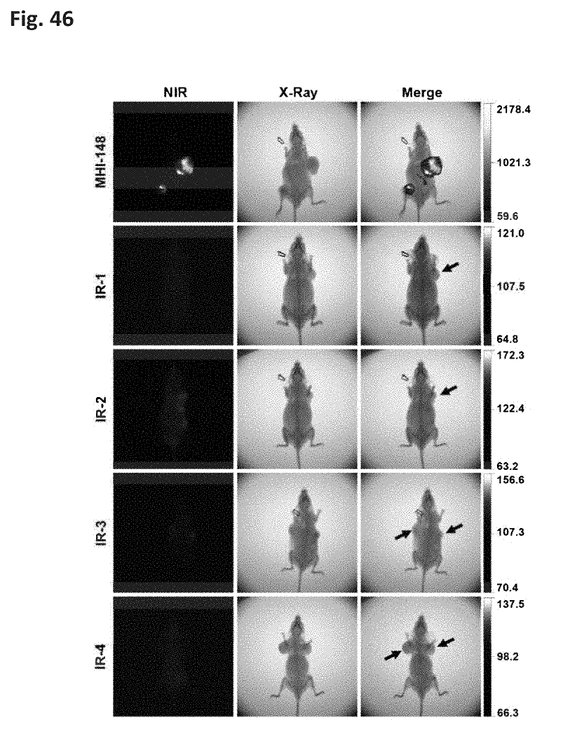

Using human cancer and normal human cell lines to study dye uptake and retention, it was found that IR-783 and MHI-148 were unique in that they had both tumor imaging and targeting properties (Table 2). A comparative analysis also uncovered several common structural features of heptamethine cyanine dyes accounting for their preferential uptake and retention by cancer cells. The dyes were classified operationally as active and inactive based upon their specific uptake and retention in cancer but not normal cells. A rigid cyclohexenyl ring in the heptamethine chain with a central chlorine atom maintains photostability, increases quantum yield, decreases photobleaching, and reduces dye aggregation in solution (1). Chemical substitution of the central chlorine atom with a thio-benzyl-amine group on the cyclohexenyl ring dramatically reduced the fluorescence intensity and eliminated their uptake by cancer cells and tumor xenografts, and so would a substitution of the side chain with hydroxyl, an ester, or an amino group rather than a charged carboxyl (i.e. MHI-148) or sulfonic acid (i.e. IR-783) moiety (see Table 2, FIG. 45 and FIG. 46).

### Example 16

### Preferential Uptake and Retention of NIR Fluorescence Dyes by Human Cancer Cells and Tumor Xenografts

Human Cancer and Normal Cell Studies:

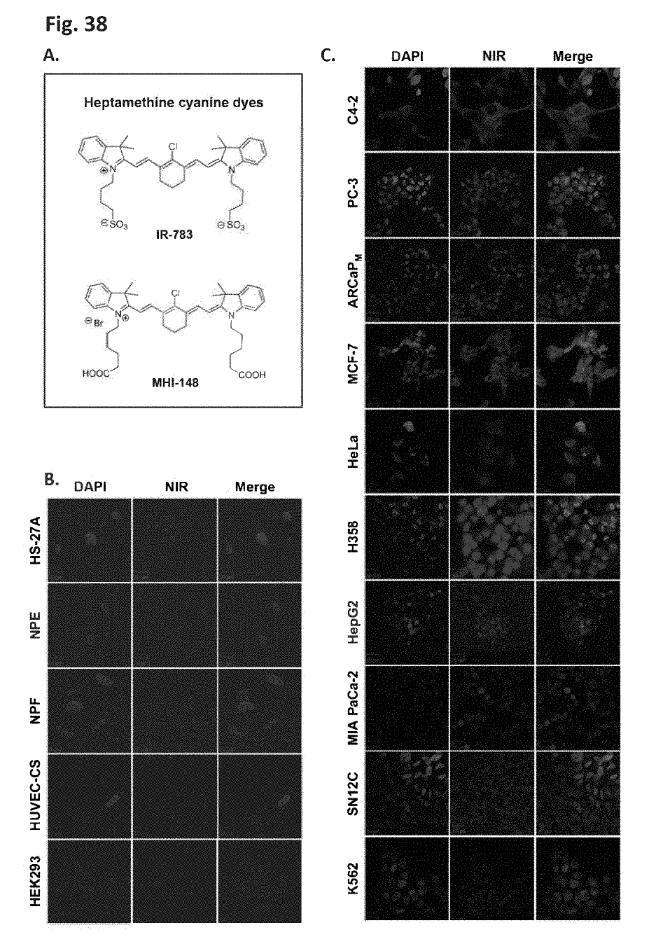

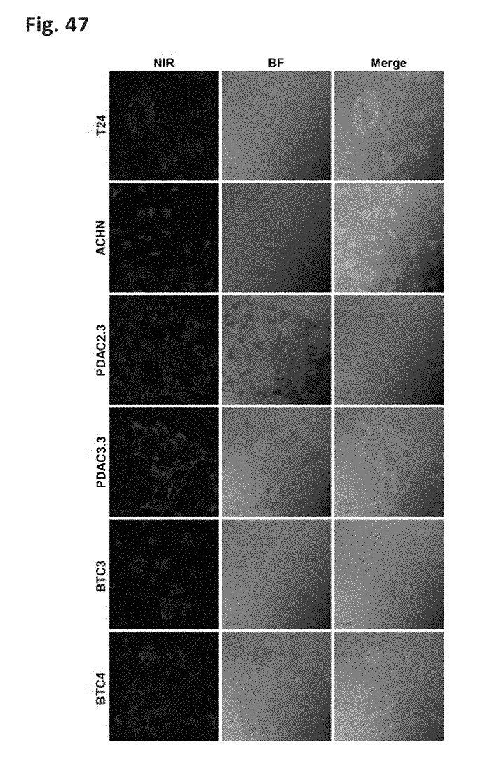

Cancer cell surface properties and surrounding leaky vasculatures have been exploited for the delivery of imaging agents (29-32). IR-783 and MHI-148 were tested for their ability to detect cancer cells (FIG. 38, panel A). The two dyes were found not to accumulate in normal human bone marrow cells (HS-27A), vascular endothelial cells (HUVEC-CS), embryonic fetal kidney cells (HEK293), a primary culture of human prostate epithelial cells (NPE), or normal prostate fibroblasts (NPF) (FIG. 38, panel B). These dyes, however, were found to be retained in cancerous cells of human origin including the prostate (C4-2, PC-3, and ARCaP_{M}), breast (MCF-7), lung (H358), cervical (HeLa), liver (HepG2), kidney (SN12C), pancreas (MIA PaCa-2), and leukemia (K562) (FIG. 38, panel C). These dyes were also found to be taken up by other malignant cells from both human and mouse, including human bladder cancer cell (T-24), renal cancer cell (ACHN), and mouse pancreatic cancer cell lines (PDAC2.3, PDAC3.3, BTC3 and BTC4 derived from transgenic mouse) (FIG. 47). There was no discernible difference in the amount and specificity of uptake of these two heptamethine cyanine NIR dyes by cancer cell lines.

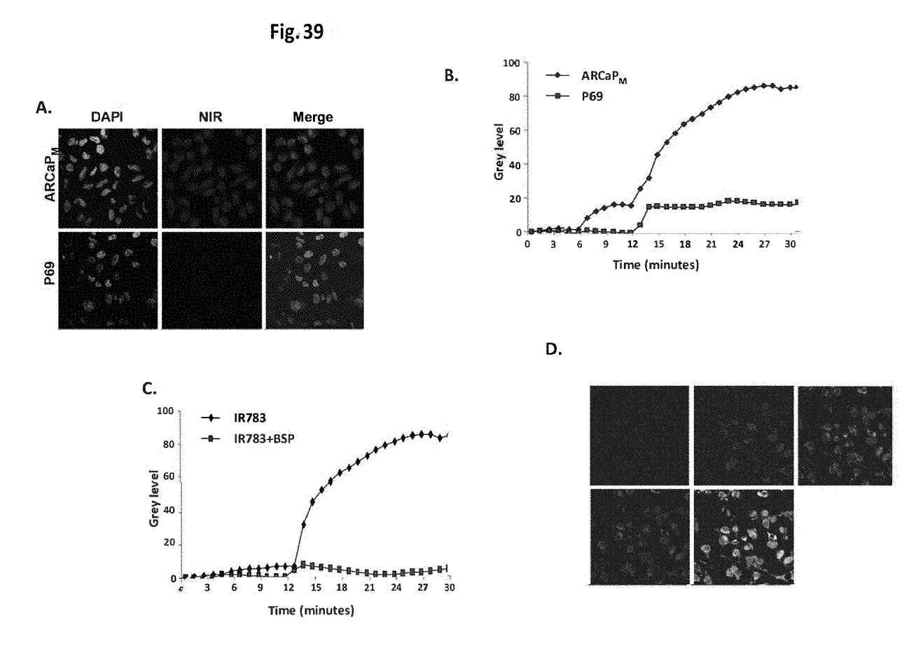

The kinetics of IR-783 uptake was compared by cultured human prostate cancer ARCaP_{M} versus P69 cells, a normal human prostate epithelial cell line (FIG. 39, panel A). This study revealed a differential time-dependent uptake and retention of IR-783 by ARCaP_{M} and P69 cells (FIG. 39B). Uptake and retention of IR-783 in ARCaP cells occurred in two phases, an early phase completed in 12 minutes, and a late phase completed in 30 minutes. In the control P69 cells, the uptake and retention of IR-783 only began at 12 minutes, with a much lower plateau. Interestingly the uptake and accumulation of IR-783 could be abolished by bromosulfophthalein (BSP), a competitive inhibitor of the organic anion transporting polypeptides (OATPs) (25) (FIG. 39, panel C). These results are consistent with the observation that IR-783 uptake into cancer cells was high at 37° C. but none at 0° C. (data not shown). These results confirmed that the cancer cell-specific uptake was an energy-dependent active process, most probably mediated by members of the OATP family.

The subcellular compartments where IR-783 was retained was evaluated. Based on the dye co-localization using the tracking dyes, the NIR signal appeared to condense on mitochondrial and lysosomal organelles, with homogenous staining also detected throughout other cytoplasmic and nuclear compartments (FIG. 39, panel D). These heptamethine cyanine NIR dyes apparently localized primarily within mitochondrial and lysosomes but can bind to a host of other intracellular proteins.

Human Tumor Xenograft Studies:

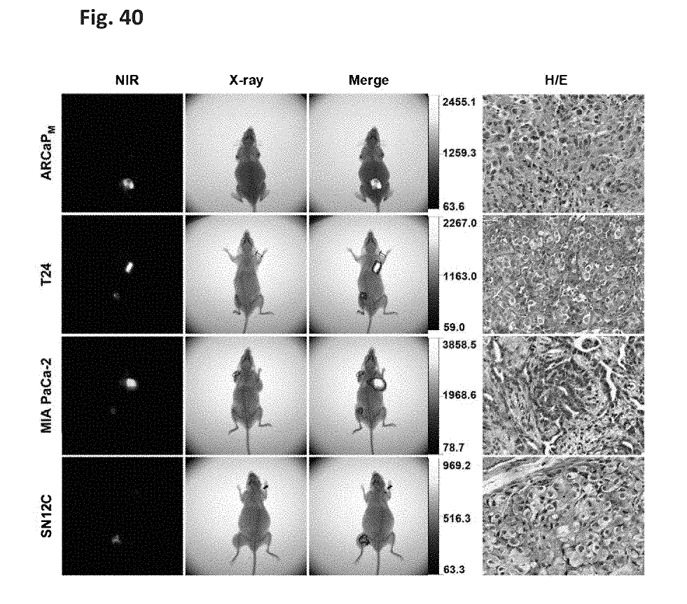

IR-783 was injected intraperitoneally (i.p.) or intravenously (i.v.) in athymic mice bearing human bladder tumors (T-24, subcutaneously), pancreas tumors (MIA PaCa-2, subcutaneously), prostate tumors (ARCaP_{M}, orthotopically), and kidney tumors (SN12C, intraosseouslly to tibia). The animals were imaged non-invasively with a NIR small animal imaging system (FIG. 40). Successive observations at different time points revealed that after the initial systemic distribution and clearance, intense signals were clearly associated with the tumors implanted at various anatomical sites, with no background interfering fluorescence from the mice. The presence of tumor cells in the tissue specimens was confirmed by histopathology analysis with tissue sections stained with H/E.

Human Cancer Metastasis Studies:

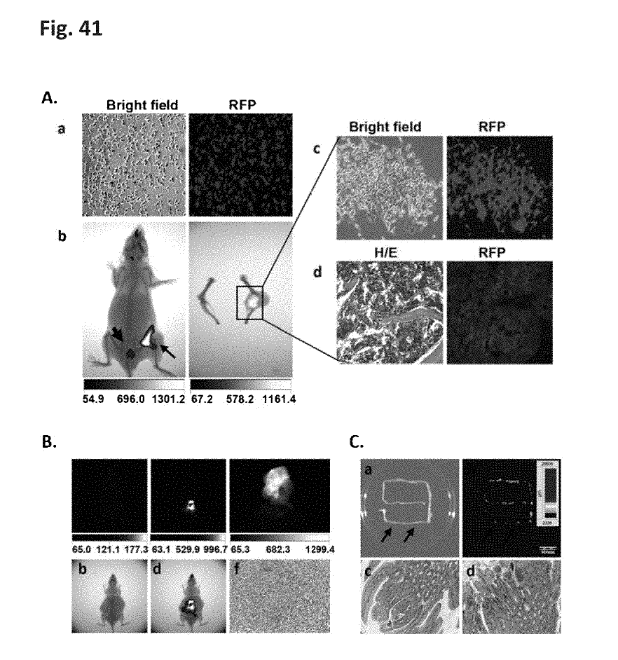

To investigate if NIR dye could detect spontaneously metastasized tumors and to confirm if the NIR dye is associated with prostate cancer bone metastasis, mice were inoculated orthotopically with ARCaP_{M} cells that were stably tagged with AsRed2 RFP (FIG. 41, panel A-a). On signs of cachexia at 3 months, the animals were subjected to non-invasive whole body NIR imaging with IR-783 (FIG. 41, panel A-b). In addition to the presence of localized orthotopic tumors (see thick arrow), RFP-tagged ARCaP_{M} tumors also appeared in mouse bone (see thin arrow). Upon ex vivo imaging, both the primary tumor and the metastases in mouse tibia/femur were detected. The presence of tumor cells in the mouse skeleton was confirmed by histopathologic evaluation and by the presence of RFP-tagged cells upon subculture of cells derived from the skeletal metastasis specimens (FIG. 41, panel A-c and FIG. 41, panel A-d).

### Example 17

### Detection of Spontaneous Prostate and Intestinal Tumors in Transgenic Mouse Models

To investigate if IR-783 could be used to detect spontaneously developed tumors, two transgenic mouse models that were known to display high degrees of tumor penetrance were adopted, the TRAMP mouse model for prostate cancer and the Apc^{Min/+} mouse model for colon cancer (33, 34). Since the TRAMP and Apc^{Min/+} mouse models represent the development of adenocarcinoma/neuroendocrine prostate tumors and adenoma of the intestine, respectively, this study also allowed assessment on whether IR-783 could detect the early stage of tumor development (i.e., adenoma). IR-783 could detect tumor in both the TRAMP mice and the Apc^{Min/+} mice (FIG. 41, panel B and FIG. 41, panel C). Specific detection of tumor but not normal cells was also confirmed by histopathologic analysis of the tumor specimens (FIG. 41, panel B-f, and FIG. 41, panel C-c and FIG. 41, panel C-d). An additional advantage of IR-783 imaging was its optical stability, even after prolonged tissue fixation. TRAMP tumor specimens retain heptamethine cyanine NIR fluorescence even after being stored in neutralized formalin solution for 3 weeks (FIG. 41, panel B-e).

### Example 18

### NIR Dye Tissue Distribution Studies

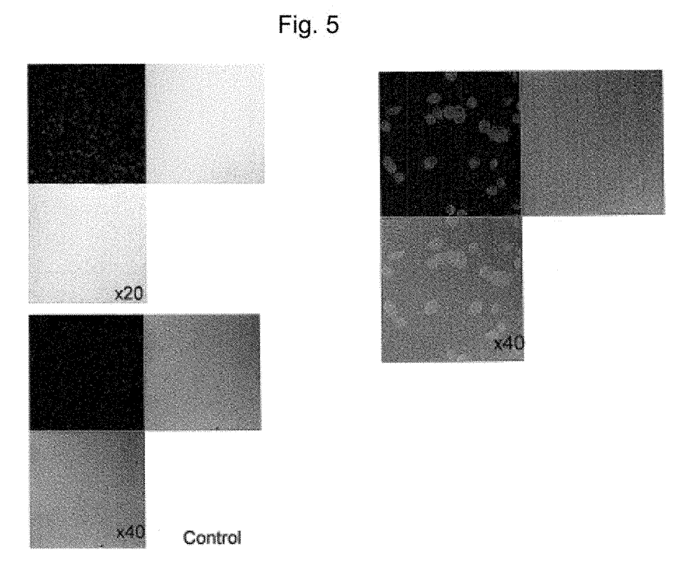

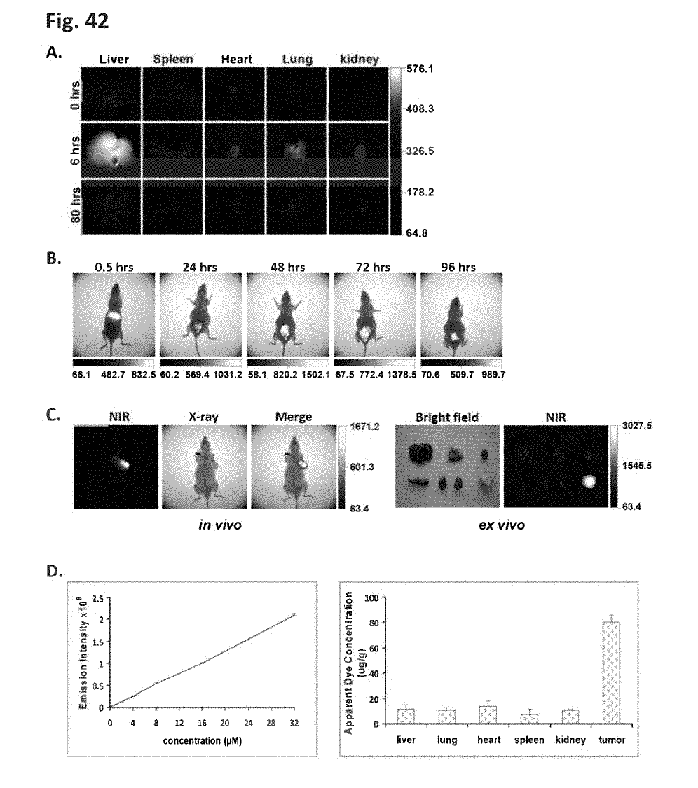

Heptamethine cyanine dye tissue distribution studies were conducted in normal and tumor-bearing mice. Time-dependent dye clearance from normal mouse organs is shown in FIG. 42, panel A. At 6 hrs, NIR dye IR-783 was found to accumulate in mouse liver, kidney, lung and heart. By 80 hrs, dye was cleared from all mouse vital organs. The dye, however, was found to accumulate in tumor tissues at 24 hrs with minimal background autofluorescence. Tumors retained IR-783 dye even at 4 days (or 96 hrs, see FIG. 5, panel B). In both in vivo whole body and ex vivo analysis, we detected signal to noise ratios exceeding 25 in tumor specimens; however, normal organs, liver, lung, heart, spleen and kidneys displayed very low signals (FIG. 42, panel C). In these studies, NIR dyes in tumor implants could be retained for as long as 15 days after dye administration (data not shown).

Prior to the quantification of the heptamethine cyanine dyes in excised tumors and normal organs, a standard curve was established by monitoring the emission profile of IR-783 at 820 nm (28, 35). Within concentration ranges from 0-40 μM, a linear correlation (r=0.9991) was found between the concentration of IR-783 and its emission intensity (left panel, FIG. 42, panel D). Using this standard curve, the apparent concentrations of the dye and its metabolites in tissues were estimated spectrophotometically. FIG. 42, panel D (right subpanel) shows that the apparent concentrations of the NIR dye and its metabolites (defined here as light emission intensity at 820 nm) in tumors were significantly higher than those in normal tissues with a difference approaching 10-fold (P<0.05, data are expressed as average±SEM of 3 determinations). This fluorescence emission could be contributed by the parental dye, its metabolites and their binding to nucleic acids and proteins (36).

In dye systemic toxicity study, no systemic toxicity of IR-783 dye was observed in normal C-57BL/6 mice and this dye also did not affect body weights of the mice. No abnormal histopathology was seen in vital organs harvested from mice at the time of sacrifice.

### Example 19

### Detection of Cancer Cells in Human Blood

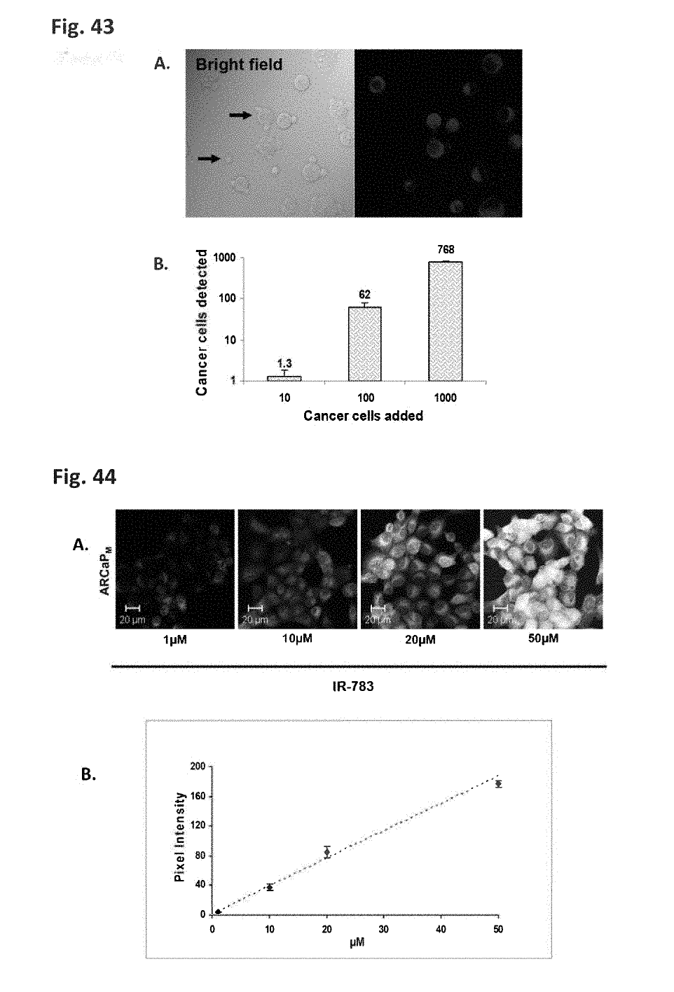

Since IR-783 was confirmed to detect human cancer but not normal cells, it was then tested whether this dye could be further exploited to detect circulating cancer cells in the blood using an experimental model. FIG. 43, panel A shows that cancer cells can be clearly visualized after mixture with human blood cells by IR-783 NIR imaging. It was estimated that this dye was sufficiently sensitive to detect as few as 10 cancer cells per milliliter in whole blood (FIG. 43, panel B).

### Example 20

### Cell Lines and Cell Culture

Human renal cancer cell lines SN12C, ACHN and Caki-1 were maintained in MEM medium (Invitrogen, Grand Island, N.Y.) supplemented with 5% fetal bovine serum (FBS) and 1% penicillin/streptomycin. The cells were maintained at 37° C. in 5% CO₂. Human embryonic kidney cells (HEK293) were cultured in RPMI 1640 medium with 5% fetal bovine serum (FBS).

### Example 21

### Cell and Tissue Uptake Study Using NIR Heptamethine Cyanine Dyes

Cells (1×10⁴/well) were seeded on vitronectin-coated four-well chamber slides (Nalgen Nunc, Naperville, Ill.) and incubated with T-medium containing 5% FBS for 24 h. After the cells had attached to the chamber slides, the cells were washed with PBS (Phosphate Buffered Saline) and exposed to the cyanine dye at a concentration of 20 μM in medium. The slides were incubated at 37° C. for 30 min, washed twice with PBS to remove excess dyes, and fixed with 10% formaldehyde at 4° C. The slides were then washed twice with PBS and covered with glass coverslips with aqueous mounting medium (Sigma-Aldrich, St. Louis, Mo.). Images were recorded by confocal laser microscopy (Zeiss LSM 510 META, Germany) equipped with 633 nm excitation laser and 670-810 nm long pass filter.

To determine the dye uptake by the mitochondria, the mitochondrial tracking dye, Mito Tracker Orange CMTMROS (Molecular Probes, Carlsbad, Calif.), was used. To determine the dye localization in lysosomes, a lysosome tracking dye, Lyso Tracker Green DND-26 (Molecular Probes, Carlsbad, Calif.), was selected. Images of mitochondrial and/or lysosome localization of the cyanine dye were merged and evidence of co-localization of the cyanine dye and tracking dyes were assessed by confocal imaging using a previously established protocol.⁶⁹

### Example 22

### Uptake and Accumulation of Cyanine Dyes in Renal Tumors in Live Mice

Human renal cancer cells (ACHN and SN12C) were implanted (1×10⁶) subcutaneously and intraosseously into 4 to 6 week old athymic nude mice, respectively. And, we also implanted Caki-1 cells into the renal capsule of athymic nude mice to establish subrenal capsule xenografts of renal cancer as previously described.⁷⁰ When tumor sizes reached between 1-6 mm in diameter, as assessed by X-ray or by palpation, mice were injected i.v. or i.p. with cyanine dyes at a dose of 11.25 μg/g or 10 nmol/20 g mouse body weight. Whole body optical imaging was taken at 24 h using a Kodak Imaging Station Imaging System 4000 MM (New Haven, Conn.) equipped with fluorescent filter sets (excitation/emission, 800/850 nm). The field of view (FOV) was 120 mm in diameter. The frequency rate for NIR excitation light was 2 mW/cm². The camera settings included maximal gain, 2×2 binning, 1024×1024 pixel resolution, and an exposure time of 5 sec. After whole body NIR imaging, the dissected organs and tumors were subjected to ex vivo imaging. Prior to imaging, mice were anesthetized with ketamine (75 mg/kg). During imaging, mice were maintained in an anesthetized state.

### Example 23

### In Vitro Fluorescence Imaging of Human Renal Tumor Tissues

Kidney tumor tissue samples were collected from 5 patients immediately after tumor nephrectomy according to an Emory approved IRB protocol. All 5 patients had histologically confirmed renal cell carcinoma of the clear cell type. The tissues were excised from tumor area, normal area and the area of tumor and normal adjacent on the kidney removed. All above procedures were conducted on ice and the excised tissues were stored at 4° C. Each incised tissue was cut into three pieces and were incubated with 20 M IR-783 dye, 20 M IR-780 and PBS, respectively, at 37° C. for 30 minutes. The dye solutions were removed and the tissues were washed five times by PBS. Optical imaging was done using Kodak Imaging Station Imaging System (see above).

To determine dye uptake in tissues, tissues cut from incised kidney samples (described above) tumor were placed in OTC medium and frozen at −80° C. These frozen tissues were retrieved for histopathologic sections cut to 5 Lm thickness for pathologic observation under a confocal microscope as described above.

### Example 24

### Detection of Renal Cancer Cells in Blood Using NIR Imaging and Flow Cytometry

Citrated whole mouse blood samples were collected from same inbred athymic nude mice. A known number of human renal cancer cells (SN12C) were added into 3 ml whole mouse blood, and mixed gently with 20 μM IR-783 and incubated for 30 minutes at 37° C. The mononuclear cells and cancer cells were recovered by gradient centrifugation using Histopaque-1077 (Sigma, St. Louis, Mo.) and then diluted with 0.5 ml of 1% paraformaldehyde to inhibit further activation. A sample incubated with PBS served as negative isotype control. The isolated live cells were observed under a Leica TCS SP confocal microscope (Leica Microsystems, Heidelberg, Germany) and samples were excited with 670 nm. All samples were analyzed within 1 hrs of collection in a Becton Dickinson LSRII flow cytometer (Becton Dickinson Biosciences, San Jose, Calif.) with a red laser diode (640 nm) for excitation and cy7 detector for detection and with LYSIS II software.

### Example 25

### Data Processing and Statistics

The statistical significance of all data was determined by Student t-test. Data were expressed as average±standard error of the mean of the indicated number of determination.

Statistical significant difference was assigned as P<0.05.

### Example 26

### Preferential Uptake of IR-783 by Human Cancer Cells

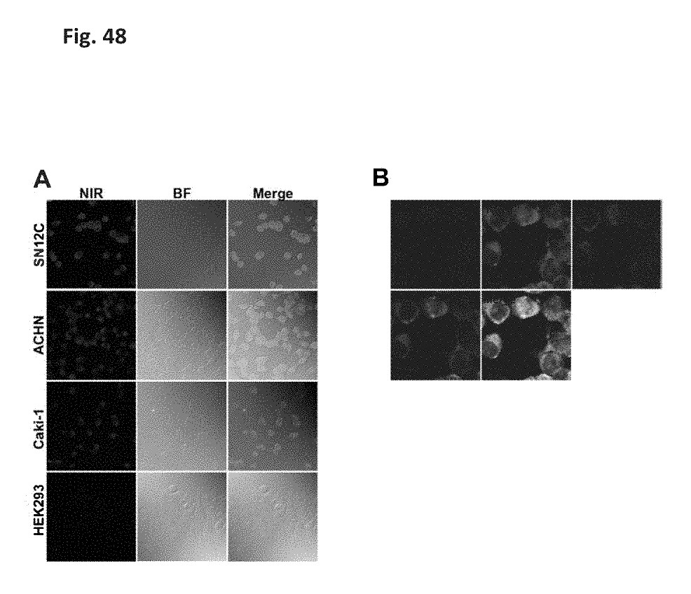

IR-783 was tested for their ability to detect cancer cells. The NIR dyes were added into culture medium to stain cancer cells of the human renal cancer cells (SN12C, ACHN and Caki-1). After removing free dyes, cells in the culture were subjected to NIR imaging. The significant uptake of IR-783 was observed in these malignant cells. In comparison, these dyes were not taken up by non-cancerous human embryonic fetal kidney cells (HEK293) (FIG. 48, panel A). The subcellular compartments in renal cancer cells where IR-783 was retained were then evaluated. The NIR signal appeared on mitochondrial and lysosome organelles. The overlay of the NIR imaging with Mito tracker imaging and Lyso tracker imaging shows nearly exact concordance in staining as evidenced by the purple to red and green colors seen in FIG. 48, panel B. This result displayed the IR-783 co-localization with mitochondrial and lysosome organelles. These heptamethine cyanine NIR dyes appears to be able to bind to a host of intracellular proteins.

### Example 27

### The Retention of IR-783 in Human Renal Tumor Xenograft Studies

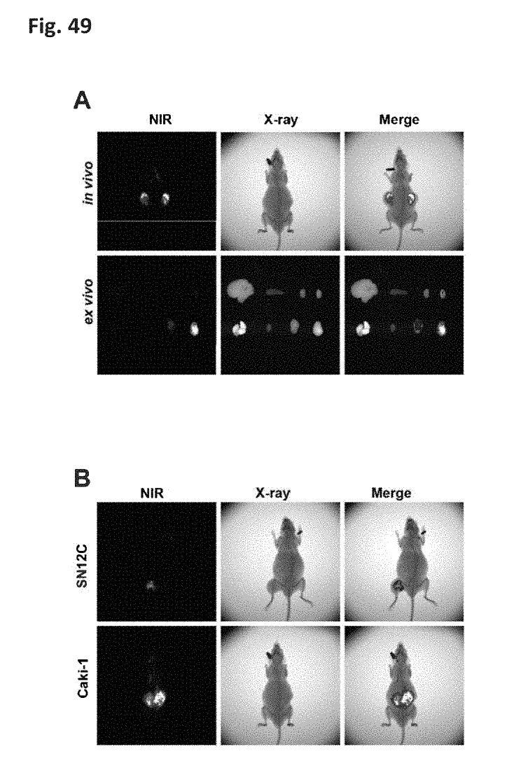

To investigate if IR-783 could detect renal tumor in vivo and ex vivo in mice, mice bearing human renal cancer subcutaneous tumors (ACHN) were subjected to Kodak System NIR Imaging after IR-783 administration. The observations revealed that after the initial systemic distribution and clearance, signals could be clearly visualized in the tumors with no background interfering fluorescence from the mice. In ex vivo analysis, higher signals in tumor specimens were detected; however, other normal organs displayed very low signals (FIG. 49, panel A). The further NIR imaging results showed that both subrenal capsule renal tumor (Caki-1) and intraosseous renal tumor (SN12C) xenografts in mice displayed strong signals at the anatomical sites where tumors were implanted (FIG. 49, panel B). In extreme cases, the inventors were able to image tumors repeatedly for up to 15 days after dye administration (data not shown).

### Example 28

### In Vitro Fluorescence Imaging of Human Renal Tumor Tissues

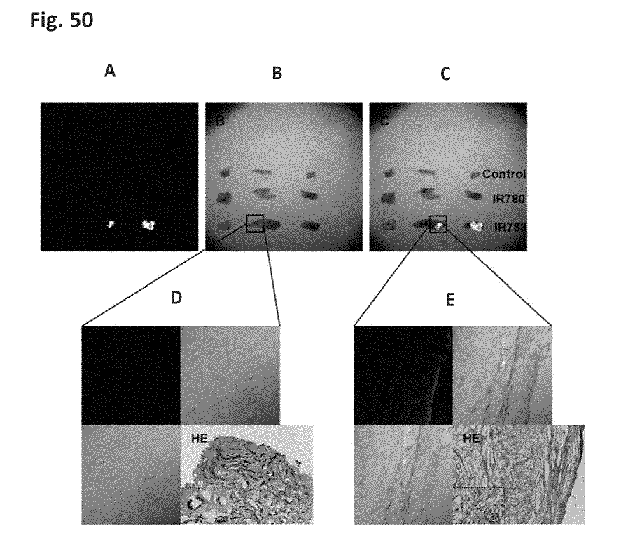

To investigate if IR-783 could be used to detect tumors in clinical samples, human kidney tumor and normal tissues excised from the clinical samples after nephrectomy were obtained. Compared with another heptamethine dye, IR-780 and PBS, IR-783 can be observed showing stronger signals in tumor tissues than the IR-780 and PBS group. Interestingly, in the samples containing normal and tumor areas, it was found that only tumor cells can take up IR-783, even the normal tissues next to the tumor can not retain the IR-783 dyes (FIG. 50, panel A and 50, panel C). The frozen tissue confocal NIR imaging confirmed that the uptake in the normal kidney tissues were undetectable while significant uptake were found in tumor tissues (FIG. 50, panel D and FIG. 50, panel E). Tumor and normal tissues are all confirmed by histopathological analysis.

### Example 29

### Detection of Renal Tumor Cells in Blood

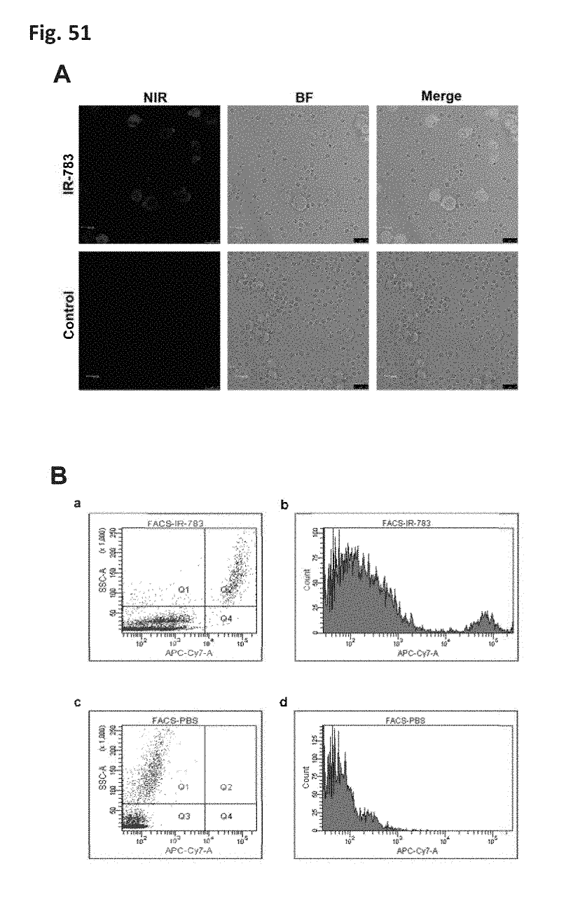

In order to employ IR-783 to detect circulating renal cancer cells in the blood due to its assured imaging and targeting properties in cancer, it was determined if this dye could differentiate renal cancer cells from normal cell using NIR imaging and a flow cytometry assay. Whole blood from mice was spiked with known numbers of cultured human renal cancer cells. The blood sample was then subjected to a brief staining with the IR-783 dye. Nucleated cells from the blood were then isolated by gradient centrifugation and subjected to confocal fluorescence microscopy and flow cytometry. FIG. 51, panel A shows that cancer cells can be clearly visualized after dye mixing with human blood, but without dye staining, the cancer cells can not be identified from normal mononuclear cells under NIR imaging. The flow cytometry results showed that the cancer cells staining IR-783 were totally identified from normal lymphocytes (see Q2 in FIG. 51, panel B-a and FIG. 51, panel B-b). Unstained samples displayed that there were no difference of dye distribution between cancer cells and lymphocytes (see FIG. 51, panel B-c and FIG. 51, panel B-d).

### Example 30

### Single Cell Detection

To perform single cell detection, 3 ml heparinized whole blood is incubated with 20 μM of a selected NIR carbocyanine dye at room temperature for 30 minutes. Mononucleated cells are isolated from the blood by gradient centrifugation. The dye uptake and stained cells are washed 2 times in phosphate buffered saline and are subjected to near-infrared detection; for example, by a microfluidics apparatus, by fluorescence microscopy, or by flow cytometric analysis, in which the stained cells are isolated and characterized.

### REFERENCES

1. Henary M, Mojzych M. Stability and reactivity of polymethine dyes in solution. Topic Heterocycl Chem, Springer Berlin/Heifelberg 2008; 14: 221-38.
2. Licha K, Riefke B, Ebert B, Grotzinger C. Cyanine dyes as contrast agents in biomedical optical imaging. Acad Radiol 2002; 9 Suppl 2: S320-2.
3. Rao J, Dragulescu-Andrasi A, Yao H. Fluorescence imaging in vivo: recent advances. Curr Opin Biotechnol 2007; 18: 17-25.
4. Frangioni J V. In vivo near-infrared fluorescence imaging. Curr Opin Chem Biol 2003; 7: 626-34.
5. Hawrysz D J, Sevick-Muraca E M. Developments toward diagnostic breast cancer imaging using near-infrared optical measurements and fluorescent contrast agents. Neoplasia 2000; 2: 388-417.
6. Ntziachristos V, Bremer C, Weissleder R. Fluorescence imaging with near-infrared light: new technological advances that enable in vivo molecular imaging. Eur Radiol 2003; 13: 195-208.
7. Gao X, Cui Y, Levenson R M, Chung L W, Nie S. In vivo cancer targeting and imaging with semiconductor quantum dots. Nat Biotechnol 2004; 22: 969-76.
8. Hintersteiner M, Enz A, Frey P, et al. In vivo detection of amyloid-beta deposits by near-infrared imaging using an oxazine-derivative probe. Nat Biotechnol 2005; 23: 577-83.
9. Wu X, Liu H, Liu J, et al. Immunofluorescent labeling of cancer marker Her2 and other cellular targets with semiconductor quantum dots. Nat Biotechnol 2003; 21: 41-6.
10. Humblet V, Lapidus R, Williams L R, et al. High-affinity near-infrared fluorescent small-molecule contrast agents for in vivo imaging of prostate-specific membrane antigen. Mol Imaging 2005; 4: 448-62.
11. Frangioni J V. New technologies for human cancer imaging. J Clin Oncol 2008; 26: 4012-21.
12. Kondepati V R, Heise H M, Backhaus J. Recent applications of near-infrared spectroscopy in cancer diagnosis and therapy. Anal Bioanal Chem 2008; 390: 125-39.
13. Pierce M C, Javier D J, Richards-Kortum R. Optical contrast agents and imaging systems for detection and diagnosis of cancer. Int J Cancer 2008; 123: 1979-90.
14. Edwards P A. Heterogeneous expression of cell-surface antigens in normal epithelia and their tumours, revealed by monoclonal antibodies. Br J Cancer 1985; 51: 149-60.
15. Heppner G H, Miller F R. The cellular basis of tumor progression. Int Rev Cytol 1998; 177: 1-56.
16. Strekowski L, Lipowska M, Patonay G. Substitution Reactions of a Nucleofugal Group in Heptamethine Cyanine Dyes. Synthesis of an Isothiocyanato Derivative for Labeling of Proteins with a Near-Infrared Chromophore. J Org Chem 1992; 57.
17. Narayanan N, Patonay G. A New Method for the Synthesis of Heptamethine Cyanine Dyes:Synthesis of New Near-Infrared Fluorescent Labels. J Org Chem 1995; 60.
18. Zhang Z, Achilefu S. Synthesis and Evaluation of Polyhydroxylated Near-Infrared Carbocyanine Molecular Probes. Organic Letters 2004; 6.
19. Thalmann G N, Sikes R A, Wu I T, et al. LNCaP progression model of human prostate cancer: androgen-independence and osseous metastasis. Prostate 2000; 44: 91-103 July 1; 44(2).
20. Zhau H Y, Chang S M, Chen B Q, et al. Androgen-repressed phenotype in human prostate cancer. Proc Natl Acad Sci USA 1996; 93: 15152-7.
21. Sung S Y, Hsieh C L, Law A, et al. Coevolution of prostate cancer and bone stroma in three-dimensional coculture: implications for cancer growth and metastasis. Cancer Res 2008; 68: 9996-10003.
22. Nomura T, Huang W C, Seo S, Zhau H E, Mimata H, Chung L W. Targeting beta2-microglobulin mediated signaling as a novel therapeutic approach for human renal cell carcinoma. J Urol 2007; 178: 292-300.
23. Marcus A I, Peters U, Thomas S L, et al. Mitotic kinesin inhibitors induce mitotic arrest and cell death in Taxol-resistant and -sensitive cancer cells. The Journal of biological chemistry 2005; 280: 11569-77.
24. Moreno R D, Ramalho-Santos J, Chan E K, Wessel G M, Schatten G. The Golgi apparatus segregates from the lysosomal/acrosomal vesicle during rhesus spermiogenesis: structural alterations. Dev Biol 2000; 219: 334-49.
25. Cui Y, Konig J, Leier I, Buchholz U, Keppler D. Hepatic uptake of bilirubin and its conjugates by the human organic anion transporter SLC21A6. The Journal of biological chemistry 2001; 276: 9626-30.
26. Wu T T, Sikes R A, Cui Q, et al. Establishing human prostate cancer cell xenografts in bone: induction of osteoblastic reaction by prostate-specific antigen-producing tumors in athymic and SCID/bg mice using LNCaP and lineage-derived metastatic sublines. Int J Cancer 1998; 77: 887-94.
27. Xu J, Wang R, Xie Z H, et al. Prostate cancer metastasis: role of the host microenvironment in promoting epithelial to mesenchymal transition and increased bone and adrenal gland metastasis. Prostate 2006; 66: 1664-73.
28. Saxena V, Sadoqi M, Shao J. Polymeric nanoparticulate delivery system for Indocyanine green: biodistribution in healthy mice. Int J Pharm 2006; 308: 200-4.
29. Corti A, Ponzoni M. Tumor vascular targeting with tumor necrosis factor alpha and chemotherapeutic drugs. Ann N Y Acad Sci 2004; 1028: 104-12.
30. Cuenod C A, Fournier L, Balvay D, Guinebretiere J M. Tumor angiogenesis: pathophysiology and implications for contrast-enhanced MRI and CT assessment. Abdom Imaging 2006; 31: 188-93.
31. Maio M, Altomonte M, Calabro L, Fonsatti E. Bioimmunotherapeutic targets on angiogenetic blood vessels in solid malignangies. Front Biosci 2001; 6: D776-84.
32. Nanda A, St Croix B. Tumor endothelial markers: new targets for cancer therapy. Curr Opin Oncol 2004; 16: 44-9.
33. Gingrich J R, Barrios R J, Morton R A, et al. Metastatic prostate cancer in a transgenic mouse. Cancer Res 1996; 56: 4096-102.
34. Ghaleb A M, McConnell B B, Nandan M O, Katz J P, Kaestner K H, Yang V W. Haploinsufficiency of Kruppel-like factor 4 promotes adenomatous polyposis coli dependent intestinal tumorigenesis. Cancer Res 2007; 67: 7147-54.
35. Johnson J L, West J K, Nelson A D, Reinhart G D. Resolving the fluorescence response of Escherichia coli carbamoyl phosphate synthetase: mapping intra- and intersubunit conformational changes. Biochemistry 2007; 46: 387-97.
36. Silva G L, Ediz V, Yaron D, Armitage B A. Experimental and computational investigation of unsymmetrical cyanine dyes: understanding torsionally responsive fluorogenic dyes. J Am Chem Soc 2007; 129: 5710-8.
37. Chen Y, Ohkubo K, Zhang M, et al. Photophysical, electrochemical characteristics and cross-linking of STAT-3 protein by an efficient bifunctional agent for fluorescence image-guided photodynamic therapy. Photochem Photobiol Sci 2007; 6: 1257-67.
38. Chandra P, Zhang P, Brouwer K L. Short-term regulation of multidrug resistance-associated protein 3 in rat and human hepatocytes. Am J Physiol Gastrointest Liver Physiol 2005; 288: G1252-8.
39. Ito A, Yamaguchi K, Tomita H, et al. Distribution of rat organic anion transporting polypeptide-E (oatp-E) in the rat eye. Invest Ophthalmol Vis Sci 2003; 44: 4877-84.
40. Bertolino C, Caputol G, Barolo C, Viscardi G, Coluccial S. Novel Heptamethine cyanine dyes with large stokes' shift for biological applications in the near infrared. J Fluoresc 2006; 16: 221-5.
41. Delaey E, van Laar F, De Vos D, Kamuhabwa A, Jacobs P, de Witte P. A comparative study of the photosensitizing characteristics of some cyanine dyes. J Photochem Photobiol B 2000; 55: 27-36.
42. Kim R B. Organic anion-transporting polypeptide (OATP) transporter family and drug disposition. Eur J Clin Invest 2003; 33 Suppl 2: 1-5.
43. Al Sarakbi W, Mokbel R, Salhab M, Jiang W G, Reed M J, Mokbel K. The role of STS and OATP-B mRNA expression in predicting the clinical outcome in human breast cancer. Anticancer Res 2006; 26: 4985-90.
44. Ballestero M R, Monte M J, Briz O, Jimenez F, Gonzalez-San Martin F, Marin J J. Expression of transporters potentially involved in the targeting of cytostatic bile acid derivatives to colon cancer and polyps. Biochem Pharmacol 2006; 72: 729-38.
45. Marzolini C, Tirona R G, Kim R B. Pharmacogenomics of the OATP and OAT families. Pharmacogenomics 2004; 5: 273-82.
46. Mikkaichi T, Suzuki T, Tanemoto M, Ito S, Abe T. The organic anion transporter (OATP) family. Drug Metab Pharmacokinet 2004; 19: 171-9.
47. Abe T, Unno M, Onogawa T, et al. LST-2, a human liver-specific organic anion transporter, determines methotrexate sensitivity in gastrointestinal cancers. Gastroenterology 2001; 120: 1689-99.
48. Monks N R, Liu S, Xu Y, Yu H, Bendelow A S, Moscow J A. Potent cytotoxicity of the phosphatase inhibitor microcystin LR and microcystin analogues in OATPIBI- and OATPIB3-expressing HeLa cells. Mol Cancer Ther 2007; 6: 587-98.
49. Lee W, Belkhiri A, Lockhart A C, et al. Overexpression of OATPIB3 confers apoptotic resistance in colon cancer. Cancer Res 2008; 68: 10315-23.
50. Muto M, Onogawa T, Suzuki T, et al. Human liver-specific organic anion transporter-2 is a potent prognostic factor for human breast carcinoma. Cancer Sci 2007; 98: 1570-6.
51. Homer M J, R. L., Krapcho M, et al.: SEER Cancer Statistics Review, 1975-2006. Bethesda, Md.: National Cancer Institute. Based on the November 2008 SEER data submission; posted to the SEER website, 2009. Available at: http://info.cancerresearch.uk.org/cancerstats/types/kidney/incidence/. Accessed on Jun. 15, 2009.
52. Lane, B. R., Rini, B. I., Novick, A. C. et al.: Targeted molecular therapy for renal cell carcinoma. Urology, 69: 3, 2007
53. Ballou, B., Fisher, G. W., Waggoner, A. S. et al.: Tumor labeling in vivo using cyanine-conjugated monoclonal antibodies. Cancer Immunol Immunother, 41: 257, 1995
54. Weissleder, R., Tung, C. H., Mahmood, U. et al.: In vivo imaging of tumors with protease-activated near-infrared fluorescent probes. Nat Biotechnol, 17: 375, 1999
55. Licha, K., Riefke, B., Ntziachristos, V. et al.: Hydrophilic cyanine dyes as contrast agents for near-infrared tumor imaging: synthesis, photophysical properties and spectroscopic in vivo characterization. Photochem Photobiol, 72: 392, 2000
56. Henary, M., Mojzych, M.: Stability and reactivity of polymethine dyes in solution. Topic Heterocycl Chem, Springer Berlin/Heifelberg, 14: 221, 2008
57. Frangioni, J. V.: In vivo near-infrared fluorescence imaging. Curr Opin Chem Biol, 7: 626, 2003
58. Hawrysz, D. J., Sevick-Muraca, E. M.: Developments toward diagnostic breast cancer imaging using near-infrared optical measurements and fluorescent contrast agents. Neoplasia, 2: 388, 2000
59. Ntziachristos, V., Bremer, C., Weissleder, R.: Fluorescence imaging with near-infrared light: new technological advances that enable in vivo molecular imaging. Eur Radiol, 13: 195, 2003
60. Gao, X., Cui, Y., Levenson, R. M. et al.: In vivo cancer targeting and imaging with semiconductor quantum dots. Nat Biotechnol, 22: 969, 2004
61. Hintersteiner, M., Enz, A., Frey, P. et al.: In vivo detection of amyloid-beta deposits by near-infrared imaging using an oxazine-derivative probe. Nat Biotechnol, 23: 577, 2005
62. Ramjiawan, B., Maiti, P., Aftanas, A. et al.: Noninvasive localization of tumors by immunofluorescence imaging using a single chain Fv fragment of a human monoclonal antibody with broad cancer specificity. Cancer, 89: 1134, 2000
63. Wu, X., Liu, H., Liu, J. et al.: Immunofluorescent labeling of cancer marker Her2 and other cellular targets with semiconductor quantum dots. Nat Biotechnol, 21: 41, 2003
64. Edwards, P. A.: Heterogeneous expression of cell-surface antigens in normal epithelia and their tumours, revealed by monoclonal antibodies. Br J Cancer, 51: 149, 1985
65. Heppner, G. H., Miller, F. R.: The cellular basis of tumor progression. Int Rev Cytol, 177: 1, 1998
66. P. V. V. Jayaweera, A. G. U. P., M. K. I. Senevirathna, P. K. D. D. P. Pitigala, and K. Tennakone: Dye-sensitized near-infrared room-temperature photovoltaic photon detectors. APPLIED PHYSICS LETTERS, 85: 5754, 2004
67. Strekowski, L., Lipowska, M., Patonay, G.: Substitution Reactions of a Nucleofugal Group in Heptamethine Cyanine Dyes. Synthesis of an Isothiocyanato Derivative for Labeling of Proteins with a Near-Infrared Chromophore. J. Org. Chem., 57, 1992
68. Narayanan, N., Patonay, G.: A New Method for the Synthesis of Heptamethine Cyanine Dyes:Synthesis of New Near-Infrared Fluorescent Labels. J. Org. Chem., 60, 1995
69. Moreno, R. D., Ramalho-Santos, J., Chan, E. K. et al.: The Golgi apparatus segregates from the lysosomal/acrosomal vesicle during rhesus spermiogenesis: structural alterations. Dev Biol, 219: 334, 2000
70. Lee, C. H., Xue, H., Sutcliffe, M. et al.: Establishment of subrenal capsule xenografts of primary human ovarian tumors in SCID mice: potential models. Gynecol Oncol, 96: 48, 2005
71. Pu, Y., Wang, W. B., Tang, G. C. et al.: Spectral polarization imaging of human prostate cancer tissue using a near-infrared receptor-targeted contrast agent. Technol Cancer Res Treat, 4: 429, 2005
72. Bugaj, J. E., Achilefu, S., Dorshow, R. B. et al.: Novel fluorescent contrast agents for optical imaging of in vivo tumors based on a receptor-targeted dye-peptide conjugate platform. J Biomed Opt, 6: 122, 2001
73. Cui, Y., Konig, J., Leier, I. et al.: Hepatic uptake of bilirubin and its conjugates by the human organic anion transporter SLC21A6. J Biol Chem, 276: 9626, 2001
74. Al Sarakbi, W., Mokbel, R., Salhab, M. et al.: The role of STS and OATP-B mRNA expression in predicting the clinical outcome in human breast cancer. Anticancer Res, 26: 4985, 2006
75. Ballestero, M. R., Monte, M. J., Briz, O. et al.: Expression of transporters potentially involved in the targeting of cytostatic bile acid derivatives to colon cancer and polyps. Biochem Pharmacol, 72: 729, 2006
76. Marzolini, C., Tirona, R. G., Kim, R. B.: Pharmacogenomics of the OATP and OAT families. Pharmacogenomics, 5: 273, 2004
77. Mikkaichi, T., Suzuki, T., Tanemoto, M. et al.: The organic anion transporter (OATP) family. Drug Metab Pharmacokinet, 19: 171, 2004
78. Touijer, K., Jacqmin, D., Kavoussi, L. R. et al.: The Expanding Role of Partial Nephrectomy: A Critical Analysis of Indications, Results, and Complications. Eur Urol, 2009
79. Tan, S. J., Yobas, L., Lee, G. Y. et al.: Microdevice for the isolation and enumeration of cancer cells from blood. Biomed Microdevices, 11: 883, 2009

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

What is claimed is:

1.-49. (canceled)

50. A method, comprising:
providing a composition comprising a near-infrared (NIR) organic carbocyanine dye;
administering composition comprising the NIR organic carbocyanine dye to a subject in need thereof; and
imaging the subject to determine the presence or absence of an increased NIR fluorescent signal relative to a background staining intensity in the body,
wherein the method identifies or detects a cancer cell, tumor or metastase in the mixture by its increased NIR fluorescent signal relative to a background staining intensity, thereby locating a cancer cell, tumor or metastase in the subject;
wherein in case of detection of a tumor or metastase, a tumor or metastase of a size of 1 mm3 or less is identified or detected; and
wherein in case of detection of cancer cells, 10 circulating cancer cells per ml of blood of the subject are detected.

51. The method of claim 50, wherein a metastase of a size of 1 mm³ or less is detected.

52. The method of claim 50, wherein cancer cells are detected at a detection level of 10 circulating cancer cells per ml of blood of the subject.

53. The method of claim 50, wherein imaging is performed over time to follow the movement of the dye staining intensity that is increased relative to the background in the subject.

54. The method of claim 50, wherein the cancer cell, tumor or metastasis is a prostate or renal cancer cell, tumor or metastasis.

55. The method of claim 50, wherein the NIR organic carbocyanine dye is a NIR heptamethine cyanine dye.

56. The method of claim 50, wherein the NIR organic carbocyanine dye is a dye of the formula below: 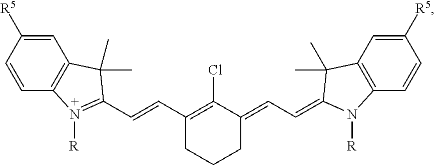
wherein R5 is H, wherein R is selected from the group consisting of (CH₂)₅COOH and (CH₂)4SO₃H, wherein R is not the same, and wherein R contains one acid and one ester group.

57. The method of claim 50, wherein the NIR organic carbocyanine dye is selected from IR-780, IR-783, and MHI-148.

58. A method, comprising:
providing composition comprising a near-infrared (NIR) organic carbocyanine dye;
administering the composition comprising the NIR organic carbocyanine dye to a subject; and
imaging a tumor in the subject over time to determine the vascularization of the tumor, or changes thereof;
wherein a strong and rapidly increasing NIR fluorescence indicates a well vascularized tumor;
wherein a weak and gradually increasing NIR fluorescence indicates a poorly vascularized tumor;
and wherein a non-fluorescent center of the tumor indicates necrotic tissue that lacks vascularization.

59. The method of claim 58, wherein the tumor is renal tumor or a tumor of the prostate.

60. The method of claim 58, wherein the NIR organic carbocyanine dye is a NIR heptamethine cyanine dye.

61. The method of claim 58, wherein the NIR organic carbocyanine dye is a dye of the formula below: 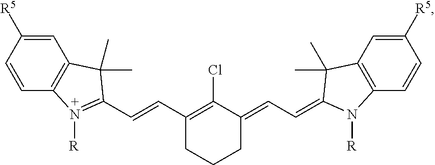
wherein R5 is H, wherein R is selected from the group consisting of (CH₂)₅COOH and (CH₂)4SO₃H, wherein R is not the same, and wherein R contains one acid and one ester group.

62. The method of claim 58, wherein the NIR organic carbocyanine dye is selected from IR-780, IR-783, and MHI-148.

63. A method, comprising:
providing a composition comprising a near-infrared (NIR) organic carbocyanine dye;
contacting the composition comprising the NIR organic carbocyanine dye to a biological sample comprising cancer or tumor cells;
imaging and observing the biological sample to differentiate live versus dead cells;
wherein live cancer or tumor cells are identified by their observed active uptake of the NIR organic carbocyanine dye; and
wherein dead cancer or tumor cells are identified by their observed lack of active uptake.

64. The method of claim 63, wherein the cancer or tumor cells are renal tumor or prostate tumor cells.

65. The method of claim 63, wherein the NIR organic carbocyanine dye is a NIR heptamethine cyanine dye.

66. The method of claim 63, wherein the NIR organic carbocyanine dye is a dye of the formula below: 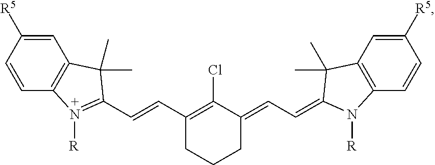
wherein R5 is H, wherein R is selected from the group consisting of (CH₂)₅COOH and (CH₂)4SO₃H, wherein R is not the same, and wherein R contains one acid and one ester group.

67. The method of claim 63, wherein the NIR organic carbocyanine dye is selected from IR-780, IR-783, and MHI-148.

68. A method, comprising:
providing a body fluid sample from a subject;
contacting the body fluid sample with a composition comprising a near-infrared (NIR) organic carbocyanine dye to form a mixture; and
analyzing the mixture to determine the presence or absence of an increased NIR fluorescent signal relative to a background staining intensity in cells contained in the body fluid sample;
wherein the analysis is performed by one or more of a microfluidic apparatus, flow cytometer, and fluorescence activated cell sorting (FACS);
wherein the method identifies or detects a cancer cell, tumor or metastase in the mixture by its increased NIR fluorescent signal; and
wherein if a cancer cell is detected, at least 1 cancer cell is identified or detected from a body fluid sample of about 10 cancer cells per ml; and
wherein if a tumor or metastase is detected, the tumor or metastase is of a size of 1 mm³ or less.

69. The method of claim 68, wherein the method characterizes a cancer cell or tumor in the body fluid sample by determining the concentration of the NIR fluorescent dye in the cancer or tumor cell.

70. The method of claim 68, additionally comprising the step of separating a detected cancer cell from the mixture by fluorescence activated cell sorting (FACS).

71. The method of claim 68, wherein the cancer cell, tumor or metastasis is a prostate or renal cancer cell, tumor or metastasis.

72. The method of claim 68, wherein the NIR organic carbocyanine dye is a NIR heptamethine cyanine dye.

73. The method of claim 68, wherein the NIR organic carbocyanine dye is a dye of the formula below: 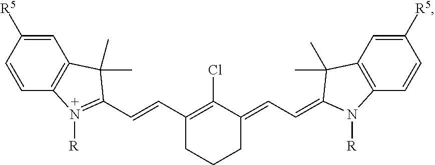
wherein R5 is H, wherein R is selected from the group consisting of (CH₂)₅COOH and (CH₂)4SO₃H, wherein R is not the same, and wherein R contains one acid and one ester group.

74. The method of claim 68, wherein the NIR organic carbocyanine dye is selected from IR-780, IR-783, and MHI-148.
